(19) United States
(12) Patent Application Publication
(10) Pub. No.: US 2011/0098278 A1
(43) Pub. Date: Apr. 28, 2011

(54) GALANTAMINE AMINO ACID AND PEPTIDE PRODRUGS AND USES THEREOF

(75) Inventors: Richard Franklin, Fleet (GB); Bernard T. Golding, Newcastle upon Tyne (GB); Robert G. Tyson, Durham (GB)

(73) Assignee: Shire LLC, Florence, KY (US)

(21) Appl. No.: 12/843,003

(22) Filed: Jul. 23, 2010

Related U.S. Application Data
(60) Provisional application No. 61/228,014, filed on Jul. 23, 2009.

(51) Int. Cl. A61K 31/55; C07D 491/06; A61P 25/28

(52) U.S. Cl. 514/215; 540/576

(57) ABSTRACT

Prodrugs of galantamine or its 3-hydroxy metabolite with amino acids or short peptides, pharmaceutical compositions containing such prodrugs and methods for treating a memory or cognition disorder with the galantamine prodrugs are provided herein. Prodrugs having side chains of valine, phenylalanine, tyrosine or para amino benzoic acid and mono-, di- and tripeptides thereof are preferred. Additionally, methods for avoiding or minimizing the adverse gastrointestinal side effects associated with galantamine administration, as well as improving the pharmacokinetics of galantamine are provided herein.

### CROSS-REFERENCE TO RELATED APPLICATIONS

This application claims benefit under 35 U.S.C. §119(e) to U.S. Provisional Application No. 61/228,014 filed on Jul. 23, 2009, which is hereby incorporated by reference in its entirety.

### FIELD OF THE INVENTION

The present invention relates to the utilization of amino acid and small peptide prodrugs of the Alzheimer drug galantamine, to minimize the gastrointestinal (GI) intolerance to the drug and enable more rapid patient titration. Additionally, improvement to the pharmacokinetics of the subsequently regenerated galantamine from the prodrug allows less frequent dosing, and improved patient compliance and response.

### BACKGROUND OF THE INVENTION

Alzheimer's disease is estimated to affect over 30 million people worldwide (Herbert L. E., (2003) Ach Neurol 60, 1119-1122 and Fact Sheet: Mental and Neurological Disorders WHO Geneva, Switzerland 2001). It is characterized by a debilitating memory loss, disorientation, impairment of language skills, declining judgment and emotional and behavioral disturbances, culminating in the inability to perform basic activities of daily living. It is caused by the deposition of β-amyloid protein plaques (Selkoe (1996). J Biol Chem 27, 18295-18298), the formation of neurofibrillary tangles (Yen et al. (1995). Neurobiol Aging 16, 3381-3387) and a loss of cortical neurons and cortical nicotinic acetyl receptors (Lamer (1995). Dementia 6, 218-224 and Zhou et al. (1995). Neurosci Letts 195, 89-92). In the UK alone, the disease currently affects nearly 700,000 people, a number expected to grow to more than 1 million by 2025 as the result of an aging population. The current total annual treatment cost in the UK for these patients is 17 billion pounds (Hone (2007). Pharma Times UK, May, 18-20).

The most common treatment strategy for Alzheimer's disease is the use of acetylcholine esterase inhibitors (AChEIs), which serve to increase brain levels of acetylcholine (ACh) to compensate for the loss of cholinergic neurons. AChEIs include doneprizil, rivastigmine, and galantamine. These drugs significantly improve cognitive function, especially in the early stages of the disease.

Galantamine, (4aS,6R,8aS)-4a,5,9,10,11,12-hexahydro-3-methoxy-11-methyl-6H-benzofuro[3a,3,2ef][2]benzazepin-6-ol hydrobromide, shown below, is a potent AChEI having in vitro IC₅₀ value of 0.36 μM (Thomson and Kewitz (1990). Life Sci 46, 129-137). Its O-desmethyl metabolite (also shown below) is even more potent, having an IC₅₀ of 0.12 μM. This metabolite also has much greater selectivity for acetylcholine esterase as compared to butyrylcholine esterase (39:1 and 200:1 respectively) (Bores et al. (1996). J Pharmacol Exp Ther, 277, 728-738). Galantamine is a particularly valuable agent having additional pharmacology believed to contribute to its actions in the treatment of Alzheimer's disease.

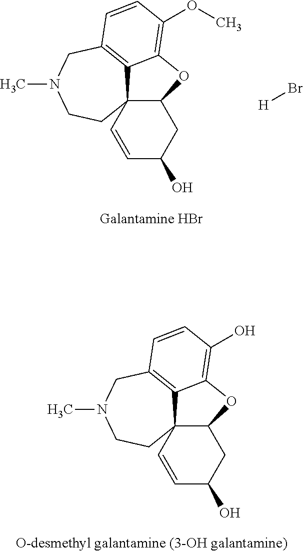

More recently, galantamine has shown utility in the treatment of autism (Nicholson et al. (2006). J Child and Adolescent Psychopharmacology 16, 621-629).

Galantamine HBr (sold by Janssen Pharmaceutica Products, L.P. as extended release capsules under the name Razadyne® ER) is available as 8 mg, 16 mg and 24 mg doses, (doses refer to the amount of galantamine free base in the composition). It is recommended to start the dosing of Razadyne® ER at 8 mg/day, and then gradually increase to the initial maintenance dose of 16 mg/day after a minimum of 4 weeks. A further increase to 24 mg/day can be done, but only after a minimum of 4 weeks at 16 mg/day (Razadyne® ER label).

In addition to being a reversible inhibitor of AChE, galantamine also functions as an allosteric nicotinic activator (Sramek et al. (2000). Expert Opin Investig Drugs 9, 2393-2402). Such stimulation of nicotinic receptors can increase the release of neurotransmitters such as ACh and glutamate. Thus, in addition to its ability to increase ACh activity via AChE inhibition, galantamine also stimulates the release of additional ACh and other transmitters via allosteric modulation of ACh effects at nicotinic cholinergic receptors.

Galantamine and other AChEI drugs, are associated with adverse gastrointestinal (GI) effects following oral administration, which include conditions affecting gut motility such as emesis (Sramek et al. (2000). Expert Opin Investig Drugs 9, 2393-2402) and diarrhea (Nordberg and Svensson (1999). Drug Safety 20, 146). Potentially stimulating gastric acid production with the consequential risk of gastric and duodenal ulceration is also a concern following oral administration of galantamine. These effects are described in the Summary of Product Characteristics (SPC) for galantamine with gastric and duodenal ulceration included in the Warnings Section. Any galantamine induced diarrhea may cause particular distress to this patient group where rectal incontinence can be a consequence of the disease progression. Approximately 24% of patients taking galantamine experience some form of nausea or vomiting, and these two adverse affects are cited as the major reason for discontinuation of drug (Sramek et al. (2000). Expert Opin Investig Drugs 9, 2393-2402). The adverse GI side effects necessitate very slow and careful upward dose titration, typically taking some 3-4 months with monthly increases of 8 mg/day up to the target of 32 mg/day. The adverse GI side-effects are not confined to galantamine, so treatment with alternative AChEIs is unlikely to offer a remedy.

There is clearly still a need for a galantamine-based pharmaceutical product with fewer GI side effects or with reduced potential to cause adverse GI side effects that enables more rapid dose titration and increased patient compliance. The present invention addresses this and other needs.

### SUMMARY OF THE INVENTION

In one embodiment of the invention, galantamine prodrugs are provided. The prodrugs comprise galantamine, or its O-demethylated metabolite, conjugated to an amino acid or peptide moiety. In a further embodiment of the invention, galantamine prodrugs of Formula 1 are provided. Formula 1 shows a generic galantamine prodrug where conjugation to an amino acid or peptide occurs through the 6-OH position, the 3-OH position, or both. The 3-OH position is functionizable in an active metabolite of galantamine, i.e., the desmethyl metabolite.

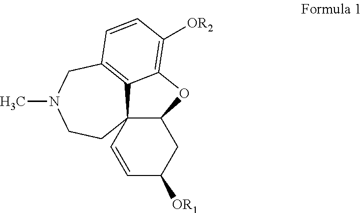

or a pharmaceutically acceptable salt thereof,

wherein,

R₁ is selected from H,

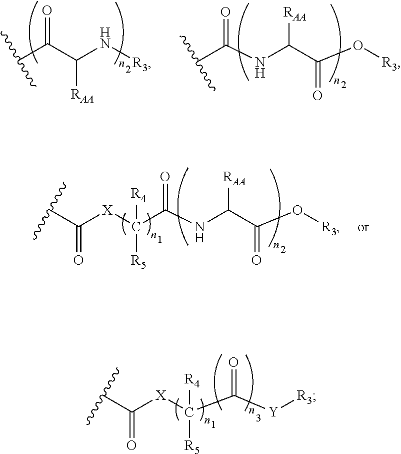

R₂ is selected from H, CH₃,

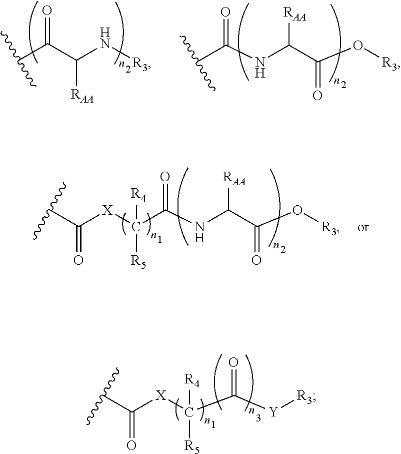

Each occurrence of R_{AA} is independently a proteinogenic or non-proteinogenic amino acid side chain;

Each occurrence of R₃ is independently selected from hydrogen, a substituted alkyl group or an unsubstituted alkyl group;

Each occurrence of R₄ and R₅ is independently selected from hydrogen,

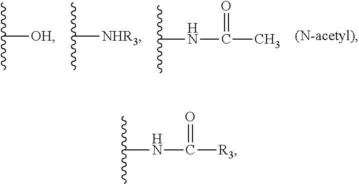

a substituted alkyl group, or an unsubstituted alkyl group;

Each occurrence of n₁ is independently an integer from 0, 1, 2, 3, 4, 5, 6, 7, 8, 9, 10, 11, 12, 13, 14, 15, or 16 and each occurrence of n₂ is independently an integer from 1, 2, 3, 4, 5, 6, 7, 8, or 9;

Each occurrence of n₃ is independently 0 or 1;

Each occurrence of X is independently (—NH—), (—O—), or absent;

Each occurrence of Y is independently

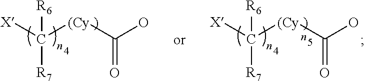

Each occurrence of X, R₆, R₇, and n₄ is as defined in the application for X, R₄, R₅, and n₁, respectively and each occurrence of n₅ is independently 0 or 1;

Each occurrence of Cy is independently a 5- or 6-membered cycloalkyl, 5- or 6-membered heterocycle, 5- or 6-membered aryl, or 5- or 6-membered heteroaryl, wherein Cy optionally has fused thereto a second ring which is a 5- or 6-membered heterocycle, 5- or 6-membered cycloalkyl 5- or 6-membered aryl or a 5- or 6-membered heteroaryl ring;

In the case of a double bond in the carbon chain defined by n₁, R₄ is present and R₅ is absent on the carbons that form the double bond; and

At least one of R₁ or R₂ is

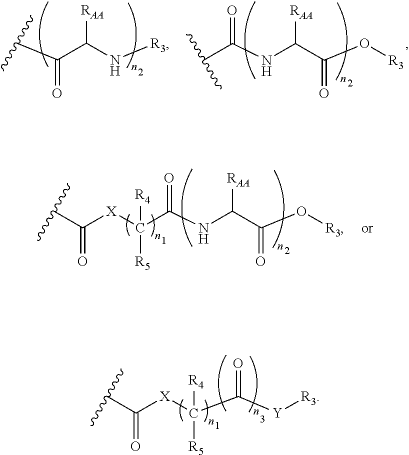

In one dicarboxylic acid linker embodiment, at least one occurrence of n₁ is 0, 1, 2, 3 or 4. In a further dicarboxylic acid linker embodiment, each occurrence of n₁ is independently 0, 1, 2, 3 or 4.

In one embodiment, each occurrence of n₂ is independently 1, 2, 3, 4, or 5.

In a preferred embodiment, the compound of the present invention has one prodrug moiety, and the prodrug moiety has one, two or three amino acids (i.e., n₂ is 1, 2, or 3), while each occurrence of R₃ is H.

In one embodiment, at least one occurrence of n₂ is 1. In another embodiment, at least one occurrence of n₂ is 2. In yet another embodiment, each occurrence of n₂ is independently 1 or 2 and each occurrence of R_{AA} is independently a proteinogenic amino acid side chain.

Compositions of the galantamine prodrug of the present invention are also provided herein. The compositions comprise at least one prodrug of the present invention (e.g., a prodrug of Formula 1), or pharmaceutically acceptable salt thereof, and at least one pharmaceutically acceptable excipient.

In one embodiment of the invention, a method for treating a disorder in a subject in need thereof with galantamine is provided. The method comprises orally administering a therapeutically effective amount of a galantamine prodrug or a pharmaceutically acceptable salt thereof to a subject in need thereof, wherein the galantamine prodrug is comprised of galantamine or its 3-OH metabolite covalently bonded to an amino acid or peptide of 2-9 amino acids in length. The disorder may be one treatable with galantamine. For example the disorder may be of memory or cognition (e.g., Alzheimer's Disease, or vascular dementia). Additional disorders of memory or cognition that may be treatable with the galantamine prodrug of the present invention may include dementia associated with Parkinson's Disease, dementia associated with Huntington's Disease, infection-induced dementia (e.g, HIV, Lyme's Disease, or Creutzfeldt-Jakob Disease), depression-induced dementia, and chronic drug use-induced dementia. Alternatively it may be used in the treatment of autism. In a further embodiment, the galantamine prodrug of the present invention has two prodrug moieties.

In another embodiment of the invention, the galantamine prodrugs provided herein confer the benefit of markedly reducing adverse gastrointestinal (GI) side effects, including nausea and vomiting, associated with oral ingestion of the parent compound. Accordingly, in another embodiment, the present invention is directed to a method for minimizing the gastrointestinal side effects normally associated with administration of galantamine. The method comprises orally administering a therapeutically effective amount of a galantamine prodrug or a pharmaceutically acceptable salt thereof, or a composition thereof, to a subject in need thereof, wherein the galantamine prodrug is comprised of galantamine or its 3-OH metabolite covalently bonded to an amino acid or peptide of 2-9 amino acids in length, and wherein upon oral administration, the prodrug or pharmaceutically acceptable salt minimizes, if not completely avoids, the gastrointestinal side effects usually seen after oral administration of the unbound galantamine. In a further embodiment, the galantamine prodrug of the present invention has two prodrug moieties.

In yet another embodiment of the invention, the amino acid and peptide prodrugs of the present invention improve galantamine's overall pharmacokinetic profile and consistency of achievement of therapeutic plasma concentrations.

In still another embodiment, a method for reducing inter- or intra-subject variability of galantamine serum levels is provided. The method comprises administering to a subject, or group of subjects, in need thereof, a therapeutically effective amount of a galantamine prodrug of the present invention (e.g., a prodrug of Formula 1), a pharmaceutically acceptable salt thereof, or a composition thereof, wherein the galantamine prodrug is comprised of galantamine or its 3-OH metabolite covalently bonded to an amino acid or peptide of 2-9 amino acids in length. The disorder may be one treatable with galantamine.

In a further embodiment, a method for sustaining plasma drug concentrations and hence reducing dosing frequency and consequently improving patient compliance is provided. Sustaining or maintaining plasma drug concentrations may result in fewer daily administrations of the galantamine prodrug, thus limiting the daily exposure of the GI tract to galantamine or the galantamine prodrug. Less daily exposure of the GI tract to galantamine or the galantamine prodrug may result in fewer GI side effects, leading to the improvement in patient compliance. The method comprises administering to a subject, or group of subjects, in need thereof, a therapeutically effective amount of a galantamine prodrug of the present invention (e.g., a prodrug of Formula 1), a pharmaceutically acceptable salt thereof, or a composition thereof, wherein the galantamine prodrug is comprised of galantamine or its 3-OH metabolite covalently bonded to an amino acid or a peptide of 2-9 amino acids in length The sustainment or maintenance of blood levels is an important feature or attribute of the galantamine prodrugs of the present invention, which allows the prolonged generation, conversion, or release of galantamine, or an active metabolite of the galantamine or an active metabolite of the galatminne prodrug from the prodrug reservoir. The active form is released into the blood to achieve sustained plasma levels of the galantamine or an active metabolite. T_{>50% Cmax}, the time or period for which the plasma drug concentration remains at or above 50% of the maximum concentration, is a useful measurement of sustainment or maintenance of blood levels.

In one embodiment, the method for achieving a sustained plasma concentration of galantamine comprises administering a galantamine prodrug of the present invention. In a further embodiment the galantamine prodrug of the present invention yields at least a 100% increase in T_{>50% Cmax} or at least a 2-fold or 3-fold greater T_{>50% Cmax} than that seen after giving the active form of the drug (i.e., a non-prodrug or parent drug).

Thus, the present invention relates to proteinogenic and/or non-proteinogenic amino acids and short-chain peptide prodrugs of galantamine or its active 3-OH metabolite. The prodrugs temporarily protect the gut from the local actions of galantamine or its active metabolite, but ultimately deliver a pharmacologically effective amount of the drug or metabolite for the improvement of cognitive function. Without wishing to be bound by any particular theory, the temporary inactivation of galantamine (or active metabolite) eliminates galantamine's direct effects on the gut, and therefore reduces the adverse GI side effects associated with its oral administration. Prodrugs of the present invention also provide a means for sustaining plasma drug levels through ongoing generation of the active agent from the prodrug. Additionally, more reproducible pharmacokinetics profiles can be achieved as the result of the active transport processes involved in prodrug absorption. These conferred attributes serve to ensure improved efficacy and better patient compliance.

These and other embodiments of the invention are disclosed or are apparent from and encompassed by the following Detailed Description.

### BRIEF DESCRIPTION OF THE DRAWINGS

FIG. 1 shows the plasma drug concentration time profile after orally dosing galantamine to the dog at 1 mg/kg.

FIG. 2 shows the plasma drug concentration time profile after orally dosing galantamine succinyl valine ester to the dog at 1 mg galantamine equivalents/kg, meaning that the dose studied contains equivalent molar amounts of the galantamine free base as administered in FIG. 1.

FIG. 3 shows the plasma drug concentration time profile after orally dosing galantamine to the monkey at 1 mg/kg

FIG. 4 shows the plasma drug concentration time profile after orally dosing galantamine succinyl valine ester to the monkey at 1 mg galantamine equivalents./kg, meaning that the dose studied contains equivalent molar amounts of the galantamine free base as administered in FIG. 3.

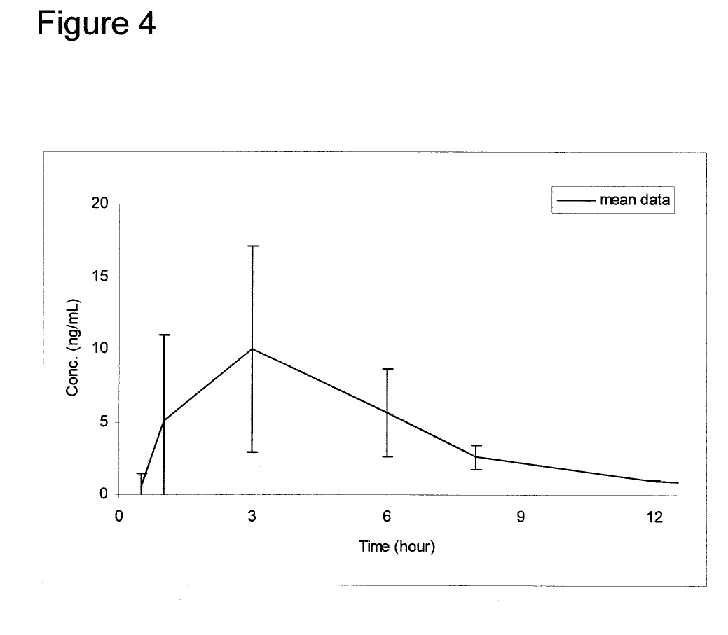

FIG. 5 shows the effects of galantamine and galantamine succinyl valine ester on rabbit stomach circular smooth muscle.

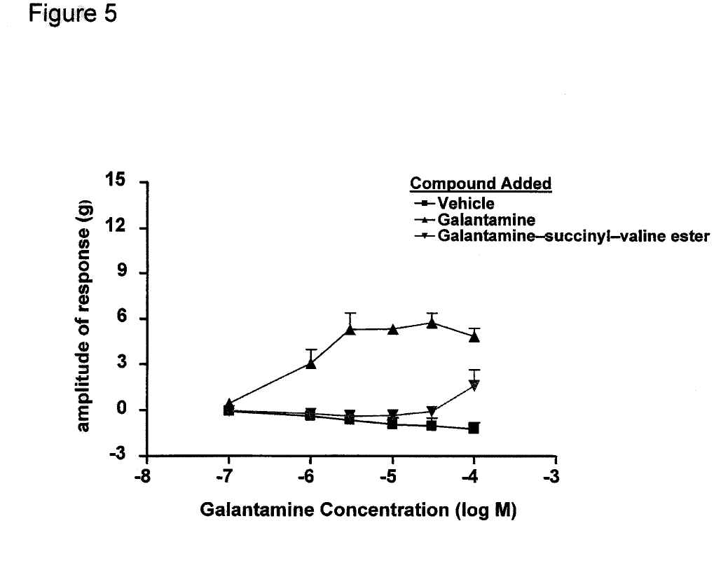

FIG. 6 shows the effects of galantamine and galantamine succinyl valine ester on human stomach circular smooth muscle.

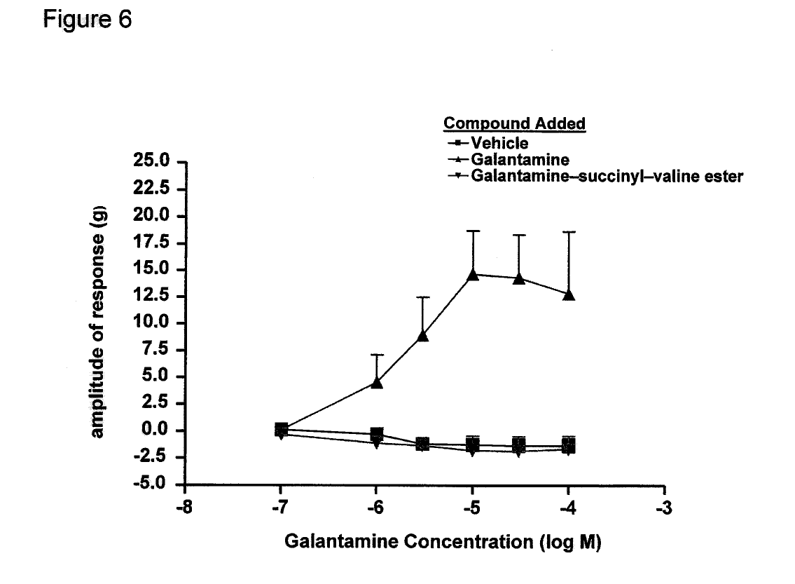

### DETAILED DESCRIPTION OF THE INVENTION

### Definitions

As used herein:

The term “peptide” refers to an amino acid chain consisting of 2 to 9 amino acids, unless otherwise specified. In preferred embodiments, the peptide used in the present invention is 2 or 3 amino acids in length. In one embodiment, a peptide can be a branched peptide. In this embodiment, at least one amino acid side chain in the peptide is bound to another amino acid (either through one of the termini or the side chain).

The term “amino acid” refers both to proteinogenic and non-proteinogenic amino acids. The amino acids contemplated for use in the prodrugs of the present invention include both proteinogenic and non-proteinogenic amino acids, preferably proteinogenic amino acids. The side chains R_{AA} can be in either the (R) or the (S) configuration. Additionally, D and/or L amino acids are contemplated for use in the present invention.

A “proteinogenic amino acid” is one of the twenty amino acids used for protein biosynthesis as well as other amino acids which can be incorporated into proteins during translation (i.e., pyrrolysine and selenocysteine). A proteinogenic amino acid generally has the formula

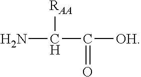

R_{AA} is referred to as the amino acid side chain, or in the case of a proteinogenic amino acid, as the proteinogenic amino acid side chain. The proteinogenic amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine, histidine, pyrrolysine and selenocysteine (see Table 1).

TABLE 1
Proteinogenic Amino Acids (Used For Protein Biosynthesis)

| Amino acid | 3 letter code | 1-letter code |
|---|---|---|
| Alanine | ALA | A |
| Cysteine | CYS | C |
| Aspartic Acid | ASP | D |
| Glutamic Acid | GLU | E |
| Phenylalanine | PHE | F |
| Glycine | GLY | G |
| Histidine | HIS | H |
| Isoleucine | ILE | I |
| Lysine | LYS | K |
| Leucine | LEU | L |
| Methionine | MET | M |
| Asparagine | ASN | N |
| Proline | PRO | P |
| Glutamine | GLN | Q |
| Arginine | ARG | R |
| Serine | SER | S |
| Threonine | THR | T |
| Valine | VAL | V |
| Tryptophan | TRP | W |
| Tyrosine | TYR | Y |
| Pyrrolysine | PYL | O |
| Selenocysteine | SEC | U |

In one embodiment, an amino acid side chain is bound to another amino acid. In a further embodiment, side chain is bound to the amino acid via the amino acid's N-terminus, C-terminus, or side chain.

Examples of proteinogenic amino acid sidechains include hydrogen (glycine), methyl (alanine), isopropyl (valine), sec-butyl (isoleucine), —CH₂CH(CH₃)₂ (leucine), benzyl (phenylalanine), p-hydroxybenzyl (tyrosine), —CH₂OH (serine), —CH(OH)CH₃ (threonine), —CH₂-3-indoyl (tryptophan), —CH₂COOH (aspartic acid), —CH₂CH₂COOH (glutamic acid), —CH₂C(O)NH₂ (asparagine), —CH₂CH₂C(O)NH₂ (glutamine), —CH₂SH, (cysteine), —CH₂CH₂SCH₃ (methionine), —(CH₂)₄NH₂ (lysine), —(CH₂)₃NHC(═NH)NH₂ (arginine) and —CH₂-3-imidazoyl (histidine).

A “non-proteinogenic amino acid” is an organic compound that is not among those encoded by the standard genetic code, or incorporated into proteins during translation. Non-proteinogenic amino acids, thus, include amino acids or analogs of amino acids other than the 22 proteinogenic amino acids used for protein biosynthesis and include, but are not limited to, the D-isostereomers of amino acids. Non proteinogenic amino acids may include non-alpha amino acids.

Examples of non-proteinogenic amino acids include, but are not limited to: para amino benzoic acid (PABA), 2-amino benzoic acid, anthranilic acid, p-hydroxybenzoic acid (PHBA), 3-amino benzoic acid, 4-aminomethyl benzoic acid, 4-amino salicylic acid (PAS), 4-amino cyclohexanoic acid 4-amino-phenyl acetic acid, 4-amino-hippuric acid, 4-amino-2-chlorobenzoic acid, 6-aminonicotinic acid, methyl-6-aminonicotinate, 4-amino methyl salicylate, 2-amino thiazole-4-acetic acid, 2-amino-4-(2-aminophenyl)-4-oxobutanoic acid (L-kynurenine), acetic acid, O-methyl serine (i.e., an amino acid side chain having the formula

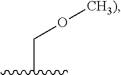

acetylamino alanine (i.e., an amino acid sidechain having the formula

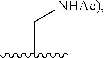

β-alanine, β-(acetylamino)alanine, β-aminoalanine, β-chloroalanine, citrulline, homocitrulline, hydroxyproline, homoarginine, homoserine, homotyrosine, homoproline, ornithine, 4-amino-phenylalanine, sarcosine, biphenylalanine, homophenylalanine, 4-nitro-phenylalanine, 4-fluoro-phenylalanine, 2,3,4,5,6-pentafluoro-phenylalanine, norleucine, cyclohexylalanine, α-aminoisobutyric acid, N-methyl-alanine, N-methyl-glycine, N-methyl-glutamic acid, tert-butylglycine, α-aminobutyric acid, α-aminoisobutyric acid, 2-aminoisobutyric acid, 2-aminoindane-2-carboxylic acid, selenomethionine, lanthionine, dehydroalanine, γ-amino butyric acid, naphthylalanine, aminohexanoic acid, phenylglycine, pipecolic acid, 2,3-diaminoproprionic acid, tetrahydroisoquinoline-3-carboxylic acid, tert-leucine, tert-butylalanine, cyclohexylglycine, diethylglycine, dipropylglycine and derivatives thereof wherein the amine nitrogen has been mono- or di-alkylated.

The term “polar amino acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

The term “nonpolar amino acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Leu (L), Val (V), Ile (I), Met (M), Gly (G) and Ala (A).

The term “aliphatic amino acid” refers to a hydrophobic amino acid having an aliphatic hydrocarbon side chain. Genetically encoded aliphatic amino acids include Ala (A), Val (V), Leu (L) and Ile (I).

The term “amino” refers to a —NH₂ group.

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl. For example, C₁₋₁₀ alkyl means a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl.

“The term “substituted alkyl” as used herein denotes alkyl radicals wherein at least one hydrogen is replaced by one more substituents such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The term “heterocycle” refers to a stable 3- to 15-membered ring radical which consists of carbon atoms and from one to five heteroatoms selected from nitrogen, phosphorus, oxygen and sulphur.

The term “cycloalkyl” group as used herein refers to a non-aromatic monocyclic hydrocarbon ring of 3 to 8 carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The terms “keto” and “oxo” are synonymous, and refer to the group ═O.

The term “carbonyl” refers to a group —C(═O).

The term “carboxyl” refers to a group —CO₂H and consists of a carbonyl and a hydroxyl group (More specifically, C(═O)OH).

The terms “carbamate group,” and “carbamate,” concerns the group

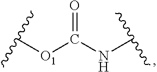

wherein the —O₁— is the phenolic oxygen in the unbound p-OH galantamine molecule. Prodrug moieties described herein may be referred to based on their amino acid or peptide and the carbamate linkage. The amino acid or peptide in such a reference should be assumed to be bound via an amino terminus on the amino acid or peptide to the carbonyl linker and galantamine, unless otherwise specified.

For example, val carbamate (valine carbamate) would have the formula

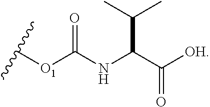

For a peptide, such as tyr-val carbamate, it should be assumed unless otherwise specified that the leftmost amino acid in the peptide is at the amino terminus of the peptide, and is bound via the carbonyl linker to galantamine to form the carbamate prodrug.

The terms “dicarboxylic acid linker” and “dicarboxyl linker,” for the purposes of the present invention, are synonymous. The dicarboxylic acid linker refers to the group between galantamine and the amino acid/peptide moiety:

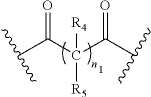

(—(CO)—(CR₄R₅)ₙ₁—(CO)—).

Alternatively, the “dicarboxylic acid linker” can have the formula:

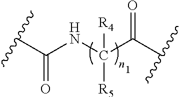

(—(CO)—(NH)—(CR₄R₅)ₙ₁—(CO—), or the formula:

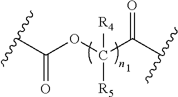

(—(CO)—(O)—(CR₄R₅)ₙ₁—(CO)—).

Regarding the dicarboxylic acid linker, one carbonyl group is bound to an oxygen atom in galantamine, while the second carbonyl is bound to the N terminus of a peptide or amino acid, or an amino group of an amino acid side chain.

Dicarboxylic acid prodrug moieties described herein may be referred to based on their amino acid or peptide and the dicarboxyl linkage. The amino acid or peptide in such a reference should be assumed to be bound via an amino terminus on the amino acid or peptide to one carbonyl (originally part of a carboxyl group) of the dicarboxyl linker while the other is attached to galantamine, unless otherwise specified. The dicarboxyl linker may or may not be variously substituted as stipulated earlier.

A non-limiting list of dicarboxylic acids for use with the present invention is given in Table 2. Although the dicarboxylic acids listed in Table 2 contain from 2 to 18 carbons, longer chain dicarboxylic acids can be used as linkers in the present invention. Additionally, the dicarboxylic acid linker can be substituted at one or more positions. A dicarboxylic acid, suitably activated, can be combined with an activated amino acid or peptide, and then reacted with an galantamine, to form a prodrug of the present invention. Prodrug syntheses procedures are discussed in more detail in the example section.

TABLE 2
Examples of Dicarboxylic Acids For
Use With The Present Invention

| Common Name | IUPAC Name | Chemical Formula |
|---|---|---|
| Oxalic Acid | Ethanedioic Acid | HOOC—COOH |
| Malonic Acid | Propanedioic Acid | HOOC—(CH₂)—COOH |
| Succinic Acid | Butanedioic Acid | HOOC—(CH₂)₂—COOH |
| Glutaric Acid | Pentanedioic Acid | HOOC—(CH₂)₃—COOH |
| Adipic Acid | Hexanedioic Acid | HOOC—(CH₂)₄—COOH |
| Pimelic Acid | Heptanedioic Acid | HOOC—(CH₂)₅—COOH |
| Suberic Acid | Octanedioic Acid | HOOC—(CH₂)₆—COOH |
| Azelaic Acid | Nonanedioic Acid | HOOC—(CH₂)₇—COOH |
| Sebacic Acid | Decanedioic Acid | HOOC—(CH₂)₈—COOH |
| Undecanedioic | Undecanedioic | HOOC—(CH₂)₉—COOH |
| Acid | Acid | |
| Dodecanedioic | Dodecanedioic | HOOC—(CH₂)₁₀—COOH |
| Acid | Acid | |
| Brassylic Acid | Tridecanedioic Acid | HOOC—(CH₂)₁₁—COOH |
| | 1,11-Undecanedi- | |
| | carboxylic Acid | |
| Tetradecanedioic | 1,12-Dodecanedi- | HOOC—(CH₂)₁₂—COOH |
| Acid | carboxylic Acid | |
| Pentadecanedioic | 1,15-Pentadecanedioic | HOOC—(CH₂)₁₃—COOH |
| Acid | Acid | |
| Thapsic Acid | Hexadecanedioic Acid | HOOC—(CH₂)₁₄—COOH |
| | Hexane-1,16-dioic Acid | |
| Heptadecanedioic | 1,15-Pentadecanedi- | HOOC—(CH₂)₁₅—COOH |
| Acid | carboxylic Acid | |
| Octadecanedioic | 1,16-Tetradecanedi- | HOOC—(CH₂)₁₆—COOH |
| Acid | carboxylic Acid | |

Dicarboxylic acid linkers of the present invention can have a nitrogen or oxygen atom bound to the first carbonyl group, i.e., X is (—NH—) or (—O—) in Formula 1, to give the linker structures

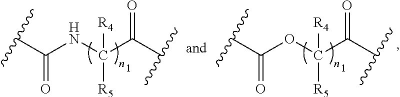

respectively. Examples of such dicarboxylic acid linkers are given in Table 2 and throughout the specification.

In one embodiment, the dicarboxylic acid linker is substituted. For example, one or more

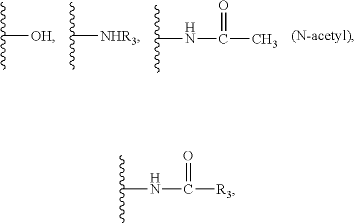

substituted alkyl groups, unsubstituted alkyl groups may be present (R₃, as defined by Formula 1). In these embodiments, X (—NH— or —O—, as defined by Formula 1) may be present or absent. Examples of dicarboxylic acid linkers are given in Table 2.

In one embodiment, the carbon chain

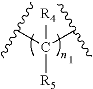

in the dicarboxylic acid linker is unsaturated, and can have one or more double bonds. In these embodiments, n₁≧2 and R₅ is absent on the two carbons that form the double bond. One example of such a linker, fumaric acid, is given in Table 3.

TABLE 3
Dicarboxylic Acid Linkers For Use With The Present Invention.

| | | Valine Prodrug Moiety |
|---|---|---|
| Dicarboxylic Acid | | (galantamine or 3-OH galantamine |
| Linker Name | Structure | oxygen shown) |
| N^{a}-Acetyl Aspartic Acid Linker | 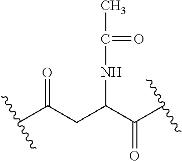 | 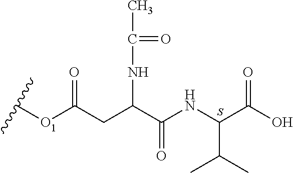 |
| N^{a}-Acetyl Glutamic Acid Linker | 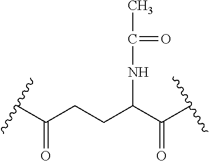 | 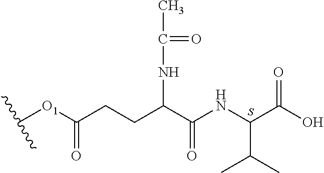 |
| Malic Acid Linker | 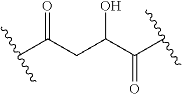 | 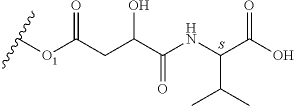 |
| Tartaric Acid Linker | 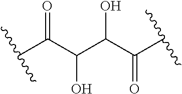 | 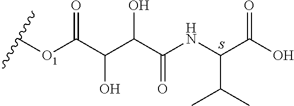 |
| Citramilic Acid Linker | 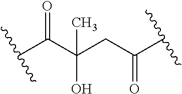 | 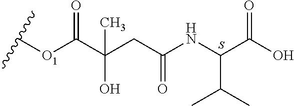 |
| β-Alanine Linker | 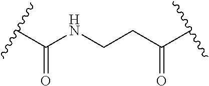 | 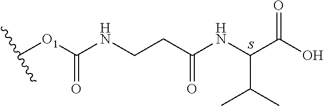 |
| γ-Aminobutyric Acid (GABA) Linker | 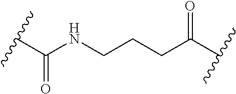 | 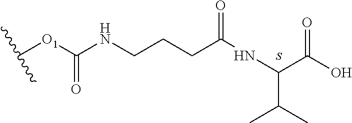 |
| 3-(Carboxyoxy) Butanoic Acid Linker | 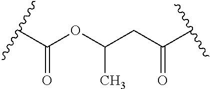 | 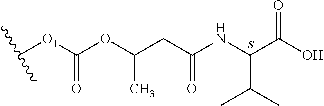 |
| 3-(Carboxyoxy) Propanoic Acid Linker | 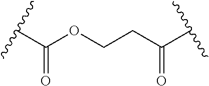 | 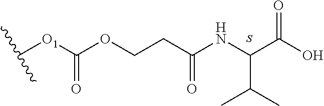 |
| 4-(Carboxyoxy) Butanoic Acid Linker | 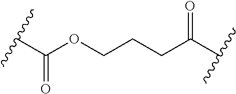 | 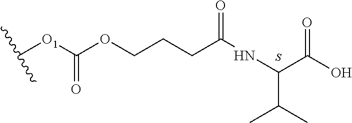 |
| Fumaric Acid Linker | 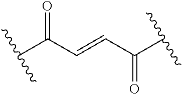 | 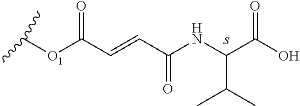 |

Examples of dicarboxylic acid prodrug moieties of the present invention include valine succinate, which has the formula

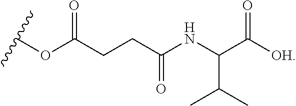

For a dipeptide, such as tyrosine-valine succinate, it should be assumed unless otherwise specified that the amino acid adjacent to the drug, in this case valine, is attached via the amino terminus to the dicarboxylic acid linker. The terminal carboxyl residue of the dipeptide (in this case tyrosine) forms the C (carboxyl) terminus.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. In some embodiments, water or aqueous-based solutions are employed as carriers for orally administered formulations. In other embodiments, oil-based carriers are employed as carrier for orally-administered formulations. Suitable pharmaceutical carriers are described in Remington's Pharmaceutical Sciences by E. W. Martin, 18^{th} Edition.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the appropriate governmental agency or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “treating” includes: (1) preventing or delaying the appearance of clinical symptoms of the state, disorder or condition developing in an animal that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a patient to be treated is either statistically significant or at least perceptible to the patient or to the physician.

The term “subject” includes humans and other mammals, such as domestic animals (e.g., dogs and cats).

The term “prodrug” means a pharmacological substance (i.e., active agent or drug) that is administered in an inactive (or significantly less active) form. The invention provides covalent attachment of galantamine and derivatives or analogs thereof to a variety of chemical moieties. The chemical moieties may include any substance which results in a prodrug form, i.e., a molecule which is converted into its active form in the body by normal metabolic processes. The chemical moieties may be for instance, amino acids, nature and non-natural peptides, dicarboxylic acid residues and combinations thereof. The galantamine prodrugs can also be characterized as conjugates in that they possess a covalent attachment. They may also be characterized as conditionally bioreversible derivatives (“CBDs”) in that the galantamine prodrug preferably remains inactive until acted upon in the body to release the galantamine from the chemical moiety.

“Effective amount” means an amount of a prodrug or composition of the present invention sufficient to result in the desired therapeutic response. The therapeutic response can be any response that a user (e.g., a clinician) will recognize as an effective response to the therapy. The therapeutic response will generally be analgesia and/or an amelioration of one or more gastrointestinal side effect symptoms that are present when galantamine in the prodrug is administered in its active form (i.e., when galantamine or 3-OH galantamine is administered alone). It is further within the skill of one of ordinary skill in the art to determine appropriate treatment duration, appropriate doses, and any potential combination treatments, based upon an evaluation of therapeutic response.

The term “active ingredient,” unless specifically indicated, is to be understood as referring to galantamine or 3-OH galantamine portion of a prodrug of the present invention, as described herein. The active ingredient is the drug part of the prodrug, which can be galantamine or a metabolite of a prodrug of the invention such as 3-OH galantamine.

The term “salts” can include acid addition salts or addition salts of free bases. Suitable pharmaceutically acceptable salts (for example, of the carboxyl terminus of the amino acid or peptide) include, but are not limited to, metal salts such as sodium potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts such as the hydrochloride, hydrobromide, sulfate, phosphate; organic acid salts such as trifluoroacetate, tartrate, and maleate salts; sulfonates such as methanesulfonate, ethanesulfonate, benzenesulfonate, p-toluenesulfonate, camphor sulfonate and naphthalenesulfonate; and amino acid salts such as arginate, gluconate, galacturonate, alaninate, asparginate and glutamate salts (see, for example, Berge, et al. “Pharmaceutical Salts,” J. Pharma. Sci. 1977; 66:1). Salts of the basic azepine nitrogen may include, but not limited to, a range of differing lipophilicities e.g TFA, HBr, HCl, tartrate, maleate, tosylate, (toluene sulphonic acid) camsylate (camphor sulphonic acid), and napsylate (naphthalene sulphonic acid). Additionally, salts of the carboxylic acid residues of the conjugated amino acid/peptide moiety can be made.

The term “bioavailability,” as used herein, generally means the rate and/or extent to which the active ingredient is absorbed from a drug product and becomes systemically available, and hence available at the site of action. See Code of Federal Regulations, Title 21, Part 320.1 (2003 ed.). For oral dosage forms, bioavailability relates to the processes by which the active ingredient is released from the oral dosage form and moves to the site of action. Bioavailability data for a particular formulation provides an estimate of the fraction of the administered dose that is absorbed into the systemic circulation. Thus, the term “oral bioavailability” refers to the fraction of a dose of galantamine given orally that is absorbed into the systemic circulation after a single administration to a subject. A preferred method for determining the oral bioavailability is by dividing the AUC of galantamine (or 3-OH galantamine) given orally by the AUC of the same galantamine (or 3-OH galantamine) dose given intravenously to the same subject, and expressing the ratio as a percent. Other methods for calculating oral bioavailability will be familiar to those skilled in the art, and are described in greater detail in Shargel and Yu, Applied Biopharmaceutics and Pharmacokinetics, 4th Edition, 1999, Appleton & Lange, Stamford, Conn., incorporated herein by reference in its entirety.

The term “T_{>50% Cmax}” is the time or period for which the plasma drug concentration remains at or above 50% of their maximum concentration. Preferably the T_{>50% Cmax} increases by at least 100%, and more preferably at least 200% or at least 300%. In other embodiments the fold increase would be at least 2-fold, at least 3-fold, at least 4-fold or at least 5-fold.

### Compounds of the Invention

In one embodiment of the present invention, the prodrugs are novel amino acid and peptide prodrugs of galantamine. Preferably, these prodrugs comprise galantamine attached either directly to a single amino acid or short peptide or through a carbamate or dicarboxylic acid bridge. The amino acid may be attached singly or as a portion of a peptide. In another embodiment of the present invention, prodrugs of the more potent and selective active metabolite O-desmethyl galantamine (3-OH galantamine) are contained as novel amino acid or peptide conjugates at either the 3-hydroxyl function or the 6-hydroxyl function or both.

These prodrugs are depicted generically in Formula 1, as follows:

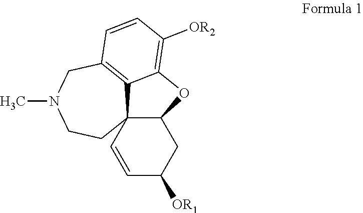

or a pharmaceutically acceptable salt thereof,

wherein,

R₁ is selected from H,

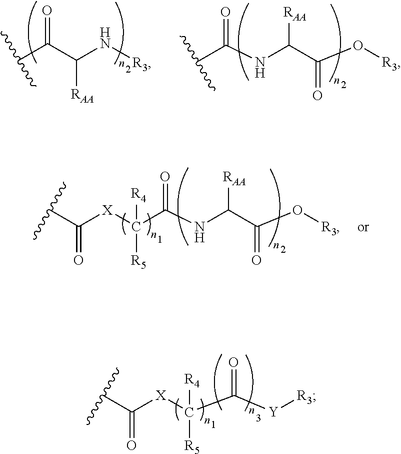

R₂ is selected from H, CH₃,

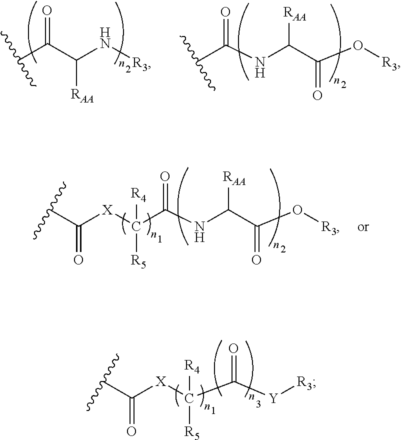

Each occurrence of R_{AA} is independently a proteinogenic or non-proteinogenic amino acid side chain;

Each occurrence of R₃ is independently selected from hydrogen, a substituted alkyl group or an unsubstituted alkyl group;

Each occurrence of R₄ and R₅ is each independently selected from hydrogen,

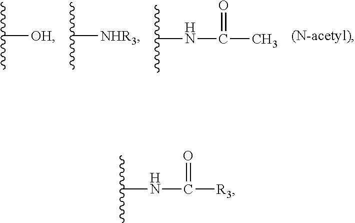

a substituted alkyl group, or an unsubstituted alkyl group;

Each occurrence of n₁ is independently an integer from 0, 1, 2, 3, 4, 5, 6, 7, 8, 9, 10, 11, 12, 13, 14, 15, or 16 and each occurrence of n₂ is independently an integer from 1, 2, 3, 4 5, 6, 7, 8, or 9;

Each occurrence of n₃ is independently 0 or 1;

Each occurrence of X is independently (—NH—), (—O—), or absent;

Each occurrence of Y is independently

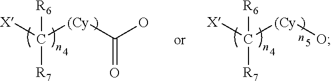

Each occurrence of X′, R₆, R₇, and n₄ is as defined in the application for X, R₄, R₅, and n1, respectively and each occurrence of n₅ is independently 0 or 1;

Each occurrence of Cy is independently a 5- or 6-membered cycloalkyl, 5- or 6-membered heterocycle, 5- or 6-membered aryl, or 5- or 6-membered heteroaryl, wherein Cy optionally has fused thereto a second ring which is a 5- or 6-membered heterocycle, 5- or 6-membered cycloalkyl 5- or 6-membered aryl or a 5- or 6-membered heteroaryl ring;

In the case of a double bond in the carbon chain defined by n₁, R₄ is present and R₅ is absent on the carbons that form the double bond; and

At least one of R₁ or R₂ is

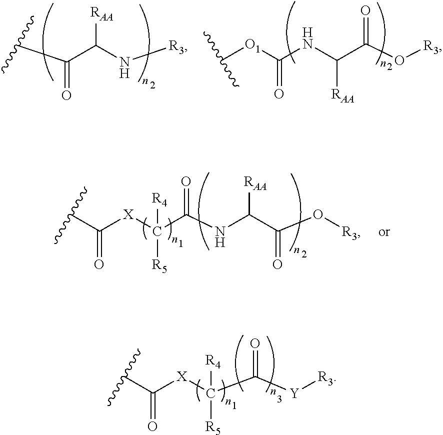

In one dicarboxylic acid linker embodiment, at least one occurrence of n₁ is 0, 1, 2, 3 or 4. In a further dicarboxylic acid linker embodiment, each occurrence of n₁ is independently 0, 1, 2, 3 or 4.

In one embodiment, each occurrence of n₂ is independently 1, 2, 3, 4, or 5.

In a preferred embodiment, the compound of the present invention has one prodrug moiety, and the prodrug moiety has one, two or three amino acids (i.e., n₂ is 1, 2 or 3), while R₃ is H.

In one embodiment, n₂ is 1. In another embodiment, n₂ is 2. In yet another embodiment, each occurrence of n₂ is independently 1 or 2 and each occurrence of R_{AA} is independently a proteinogenic amino acid side chain.

In another embodiment of the invention, prodrugs of galantamine are provided as shown in Formulae 1a-1h, below. In these embodiments, each occurrence of R_{AA}, R₃, R₄, R₅, R₆, R₇, n₁, n₂, n₃, n₄, nₛ, X, X′, and Y are defined as provided for Formula 1.

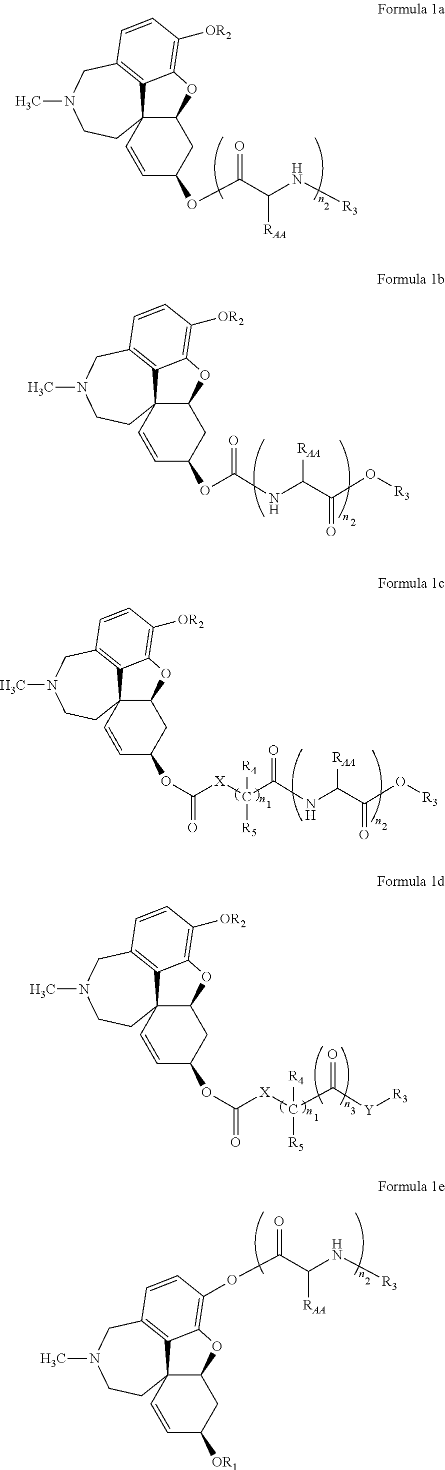

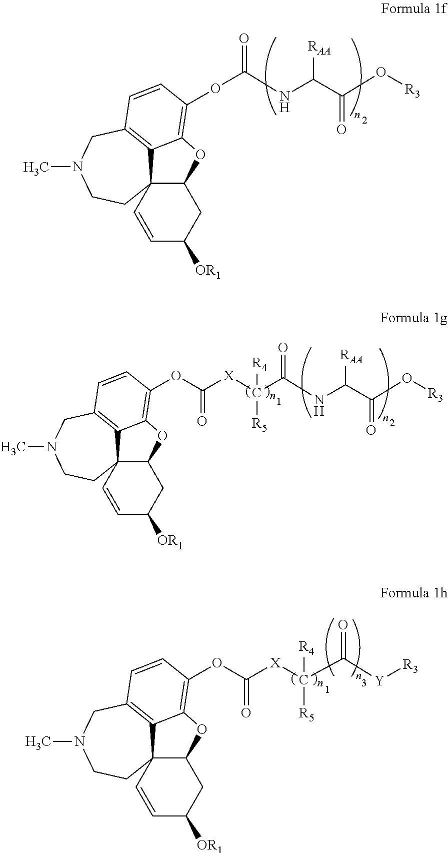

In one embodiment (i.e., an embodiment of any of Formulae 1a-1h), each occurrence of n₁ is independently 1, 2, 3 or 4. In a further embodiment, each occurrence of R₃ is H. In yet a further embodiment, each occurrence of R_{AA} is independently a proteinogenic amino acid side chain. In another embodiment (i.e., an embodiment of any of Formula 1a-1h), each occurrence of n₂ is independently 1, 2, 3 or 4. In a further embodiment, each occurrence of R₃ is independently an alkyl group. In an even further embodiment, each occurrence of R_{AA} is independently a non-proteinogenic amino acid side chain.

In another embodiment (i.e., an embodiment of any of Formulae 1a-1h), each occurrence of n₂ is independently 1, 2, 3 or 4. In a further embodiment, each occurrence of R₃ is H. In another embodiment (i.e., an embodiment of any of Formulae 1a-1h), each occurrence n₂ is independently 1, 2, 3 or 4. In a further embodiment, each occurrence R₃ is independently an alkyl group.

In yet another Formulae 1a-1h embodiment, each occurrence of n₂ is independently 1 or 2 and each occurrence of R_{AA} is independently a proteinogenic amino acid side chain.

In one Formulae 1a-1h embodiment, each occurrence of n₂ is independently 1 or 2 and at least one occurrence of R_{AA} is independently a non-proteinogenic amino acid side chain.

In one Formulae 1a-1h embodiment, each occurrence of n₁ is independently 0, 1, 2, 3 or 4. In a further embodiment, R₃ is H and each occurrence of n₂ is independently 1, 2 or 3.

In one Formulae 1a-1h embodiment, each occurrence of n₁ is independently 0, 1, 2 or 3. In a further embodiment, each occurrence of n₁ is independently 0, 1, 2 or 3 while each occurrence of each occurrence of R₃, R₄ and R₅ is hydrogen.

In one Formulae 1a-1h embodiment, each occurrence of n₁ is independently 0, 1, 2 or 3, each occurrence of n₂ is independently 1, 2 or 3 and each occurrence of R₃, R₄ and R₅ is each H. In a further embodiment, n₁ is 2.

In one Formulae 1a-1h embodiment, each occurrence of n₁ is independently 0, 1, 2 or 3, each occurrence of n₂ is independently 1, 2 or 3 and each occurrence of R₃, R₄ and R₅ is each H. In a further embodiment, n₁ is 2 and n₂ is 1.

In one Formulae 1a-1h embodiment, each occurrence of n₁ is independently 0, 1, 2 or 3, each occurrence of n₂ is independently 1, 2 or 3 and each occurrence of R₃, R₄ and R₅ is each H. In a further embodiment, n₁ is 2.

In another Formulae 1a-1h embodiment, each occurrence of n₁ is independently 1, 2 or 3 and each occurrence of n₂ is independently 1, 2 or 3. In a further embodiment, at least one occurrence of R₄ is

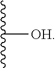

In yet another Formulae 1a-1h embodiment, each occurrence of n₁ is independently 1 or 2 and each occurrence of n₂ is independently 1, 2, 3, 4 or 5. In a further embodiment, each occurrence of R_{AA} is independently a proteinogenic amino acid side chain.

In one Formulae 1a-1h embodiment, each occurrence of n₁ is independently 0, 1 or 2, each occurrence of n₂ is independently 1 or 2 and R₃ is H. In a further embodiment, at least one occurrence of R₄ is

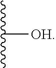

In another Formulae 1a-1h embodiment, each occurrence of n₁ is independently 0, 1 or 2, each occurrence of n₂ is independently 1 or 2 and R₃ is H. In a further embodiment, at least one occurrence of R₄ is

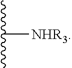

In a preferred Formulae 1a-1h embodiment, the moiety of the present invention has one or two amino acids (i.e., n₂ is 1 or 2). In one embodiment, each occurrence of n₁ is independently 1 or 2 while each occurrence of n₂ is independently 1, 2 or 3.

In a preferred Formulae 1a-1h embodiment, each occurrence of n₂ is independently 1, 2 or 3 while each occurrence of R₃, R₄ and R₅ is H. In another embodiment, n₂ is 1. In yet another Formulae 1a-1h embodiment, n₂ is 2. In yet another Formulae 1a-1b embodiment, each occurrence of n₂ is independently 1 or 2 and each occurrence of R_{AA} is independently a proteinogenic amino acid side chain.

In a further Formulae 1a-1h, each occurrence of R_{AA} is independently a non-proteinogenic amino acid side chain or a combination of proteinogenic and non-proteinogenic amino acid side chain.

In another embodiment of the invention, carbamate prodrugs of galantamine are provided, shown in Formulae 2, 3, and 4, below. In these embodiments, each occurrence of R₃, R_{AA}, and n₂ is defined as provided for Formula 1.

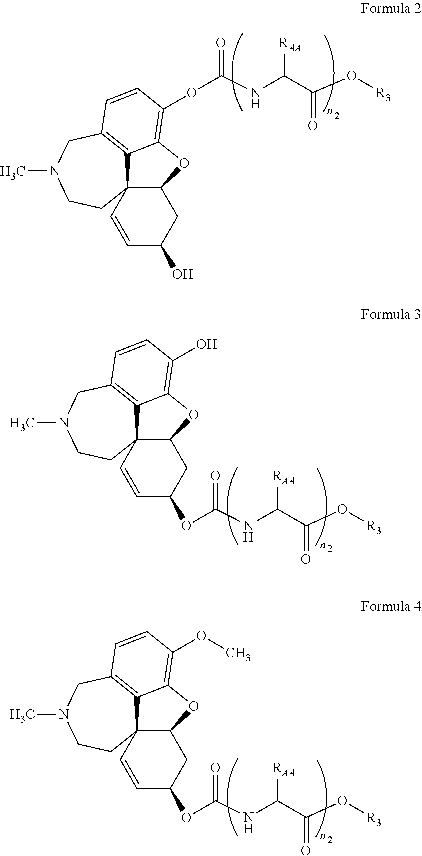

In one carbamate prodrug embodiment (i.e., an embodiment of any of Formulae 2, 3 or 4), each occurrence of n₂ is independently 1, 2, 3 or 4. In a further embodiment, R₃ is H. In yet a further embodiment, each occurrence of R_{AA} is independently a proteinogenic amino acid side chain. In another carbamate prodrug embodiment (i.e., an embodiment of any of Formula 2, 3, 4), each occurrence of n₂ is independently 1, 2, 3 or 4. In a further embodiment, each occurrence of R₃ is independently an alkyl group. In an even further embodiment, each occurrence of R_{AA} is independently a proteinogenic amino acid side chain. In another embodiment, each occurrence of R_{AA} is independently a non-proteinogenic amino acid side chain.

In another carbamate prodrug embodiment (i.e., an embodiment of any of Formulae 2, 3 or 4), each occurrence of n₂ is independently 1, 2, 3 or 4. In a further embodiment, each occurrence of R₃ is H. In another carbamate prodrug embodiment (i.e., an embodiment of any of Formulae 2, 3 or 4), each occurrence of n₂ is independently 1, 2, 3 or 4. In a further embodiment, each occurrence of R₃ is independently an alkyl group.

In yet another Formulae 2-4 embodiment, each occurrence of n₂ is independently 1 or 2 and each occurrence of R_{AA} is independently a proteinogenic amino acid side chain.

In one Formulae 2-4 embodiment, each occurrence of n₂ is independently 1 or 2 and at least one occurrence of R_{AA} is independently a non-proteinogenic amino acid side chain.

Examples of dicarboxylic acid linked galantamine prodrugs are provided in Formulae 5-13, below. In these embodiments, each occurrence of R₃, R₄, R₅, R_{AA}, n1 and n₂ is defined as provided for Formula 1. For the purposes of clarity, the galantamine phenolic oxygen atom attached to the prodrug moiety is drawn as —O₁—.

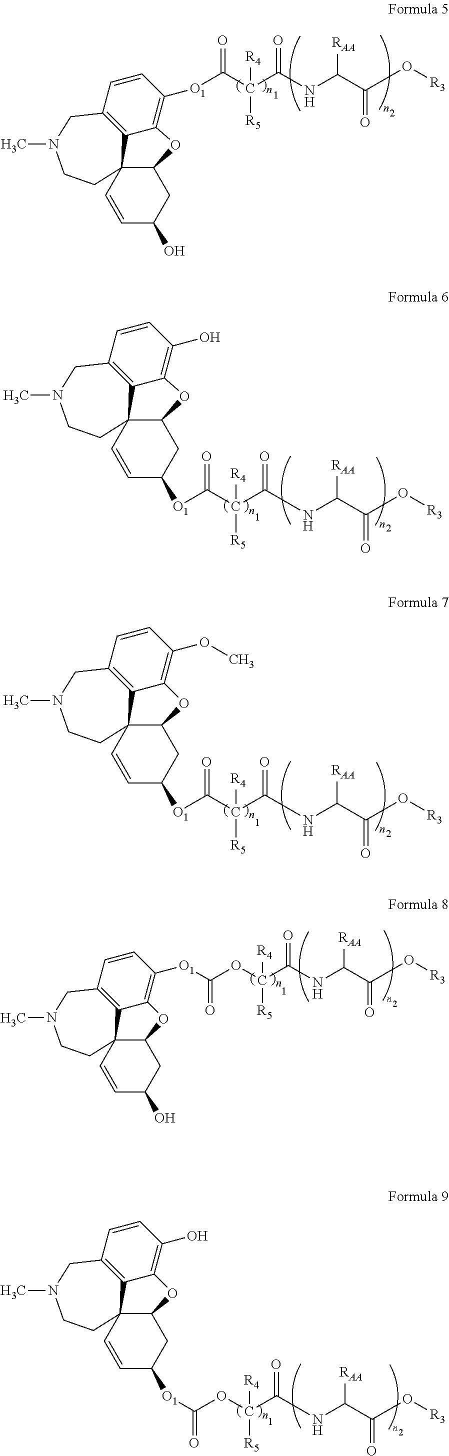

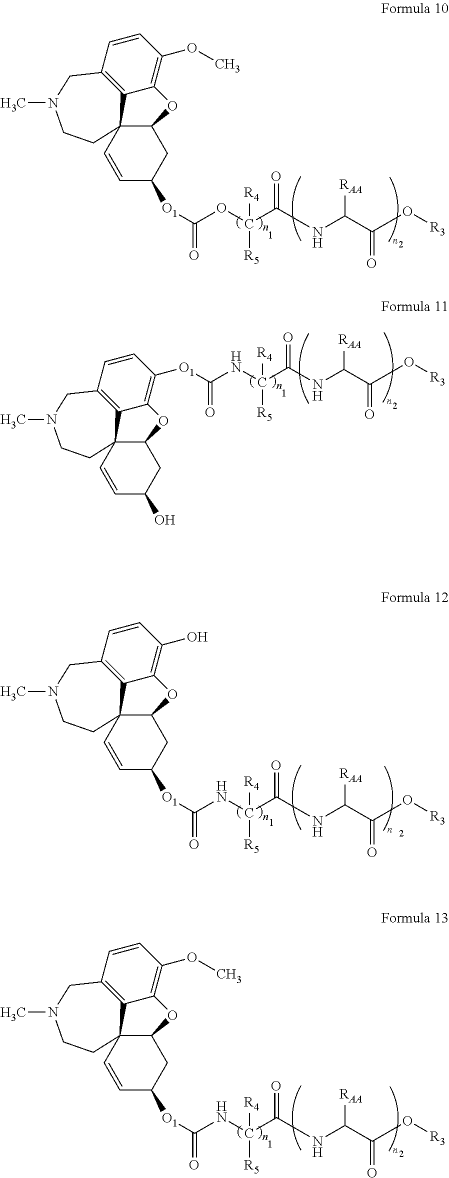

In one Formulae 5-13 embodiment, each occurrence of n₁ is independently 0, 1, 2, 3, or 4. In a further embodiment, each occurrence of R₃ is H and each occurrence of n₂ is independently 1, 2 or 3.

In one Formulae 5-13 embodiment, each occurrence of n₁ is independently 0, 1, 2 or 3. In a further embodiment, each occurrence of n₁ is independently 0, 1, 2 or 3 while each occurrence of each occurrence of R₃, R₄ and R₅ is hydrogen.

In one Formulae 5-13 embodiment, each occurrence of n₁ is independently 0, 1, 2 or 3, each occurrence of n₂ is independently 1, 2 or 3 and each occurrence of R₃, R₄ and R₅ is each H. In a further embodiment, each occurrence of n₁ is 2.

In one Formulae 5-13 embodiment, each occurrence of n₁ is independently 0, 1, 2 or 3, each occurrence of n₂ is independently 1, 2 or 3 and each occurrence of R₃, R₄ and R₅ is each H. In a further embodiment, each occurrence of n₁ is 2 and each occurrence of n₂ is 1.

In one Formulae 5-13 embodiment, each occurrence of n₁ is independently 0, 1, 2 or 3, each occurrence of n₂ is independently 1, 2 or 3 and each occurrence of R₃, R₄ and R₅ is H. In a further embodiment, each occurrence of n₁ is 2.

In another Formulae 5-13 embodiment, each occurrence of n₁ is independently 1, 2 or 3 and each occurrence of n₂ is independently 1, 2 or 3. In a further embodiment, at least one occurrence of R₄ is

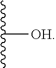

In yet another Formulae 5-13 embodiment, each occurrence of n₁ is independently 1 or 2 and each occurrence of n₂ is independently 1, 2, 3, 4 or 5. In a further embodiment, each occurrence of R_{AA} is independently a proteinogenic amino acid side chain.

In one Formulae 5-13 embodiment, each occurrence of n₁ is independently 0, 1 or 2, each occurrence of n₂ is independently 1, or 2 and each occurrence of R₃ is H. In a further embodiment, at least one occurrence of R₄ is

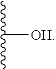

In another Formulae 5-13 embodiment, each occurrence of n₁ is independently 0, 1 or 2, each occurrence of n₂ is independently 1 or 2 and each occurrence of R₃ is H. In a further embodiment, at least one occurrence of R₄ is

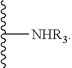

In a preferred Formulae 5-13 embodiment, the prodrug moiety of the present invention has one or two amino acids (i.e., n₂ is 1 or 2). In one embodiment, each occurrence of n₁ is independently 1 or 2 while each occurrence of n₂ is independently 1, 2 or 3.

In a preferred Formulae 5-13 embodiment, each occurrence of n₂ is independently 1,2 or 3 while each occurrence of R₃, R₄ and R₅ is H. In another embodiment, each occurrence of n₂ is 1. In yet another Formulae 5-13 embodiment, each occurrence of n₂ is 2. In yet another Formulae 5-13 embodiment, each occurrence of n₂ is independently 1 or 2 and each occurrence of R_{AA} is independently a proteinogenic amino acid side chain.

In yet another embodiment of the invention, prodrugs of Formulae 14-16 are provided. In these embodiments, each occurrence of R₃, R_{AA} and n₂ is defined as provided for Formula 1. For the purposes of clarity, the galantamine phenolic oxygen atom attached to the prodrug moiety is drawn as —O₁—.

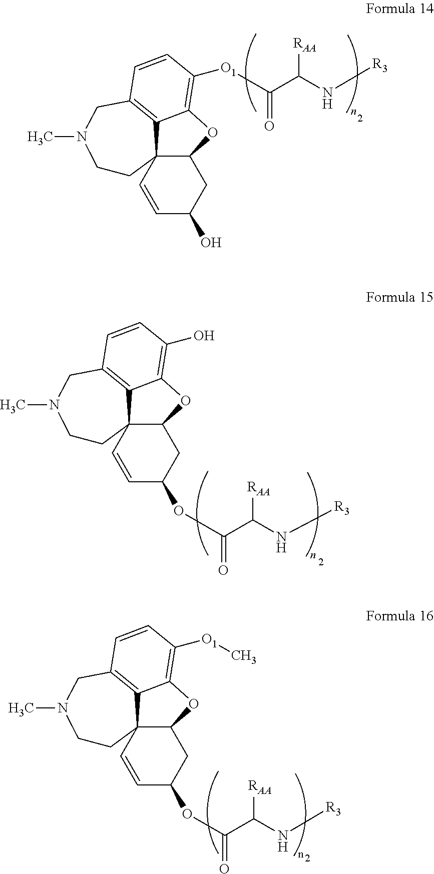

In one Formulae 14-16 embodiment, each occurrence of R₃ is H and each occurrence of n₂ is independently 1, 2 or 3. In a further embodiment, each occurrence of n₂ is 2.

In another Formulae 14-16 embodiment, each occurrence of n₂ is independently 1, 2 or 3. In a further embodiment, each occurrence of R_{AA} is independently a proteinogenic amino acid side chain.

In another Formulae 14-16 embodiment, each occurrence of n₂ is independently 1 or 2 and R₃ is H.

In a preferred Formulae 14-16 embodiment, the prodrug moiety of the present invention has one or two amino acids (i.e., n₂ is 1 or 2).

In a preferred Formulae 14-16 embodiment, each occurrence of n₂ is 1. In yet another Formulae 14-16 embodiment, each occurrence of n₂ is 2. In yet another Formulae 14-16 embodiment, each occurrence of n₂ is independently 1 or 2 and each occurrence of R_{AA} is independently a proteinogenic amino acid side chain.

Still other embodiments of the present invention are directed to prodrugs of galantamine that include two prodrug moieties. For example, in one embodiment, the present invention is directed to a prodrug with two dicarboxylic acid moieties, shown below in Formulae 17-25. In these embodiments, each occurrence of R₃, R₄, R₅, R_{AA}, n₁ and n₂ is defined as provided for Formula 1. For the purposes of clarity, the galantamine phenolic oxygen atom attached to the prodrug moiety is drawn as —O₁—.

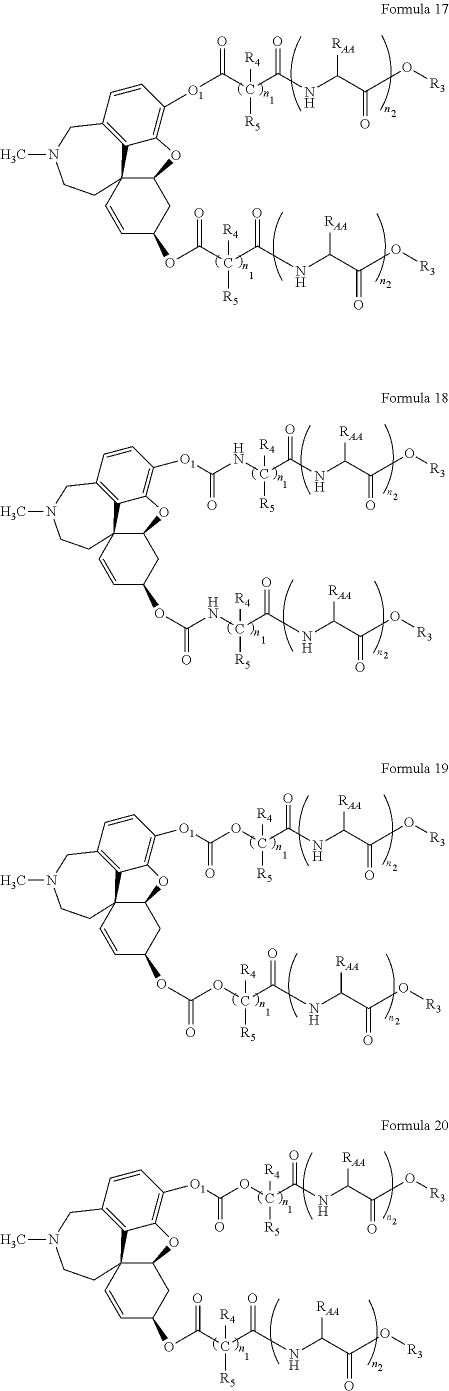

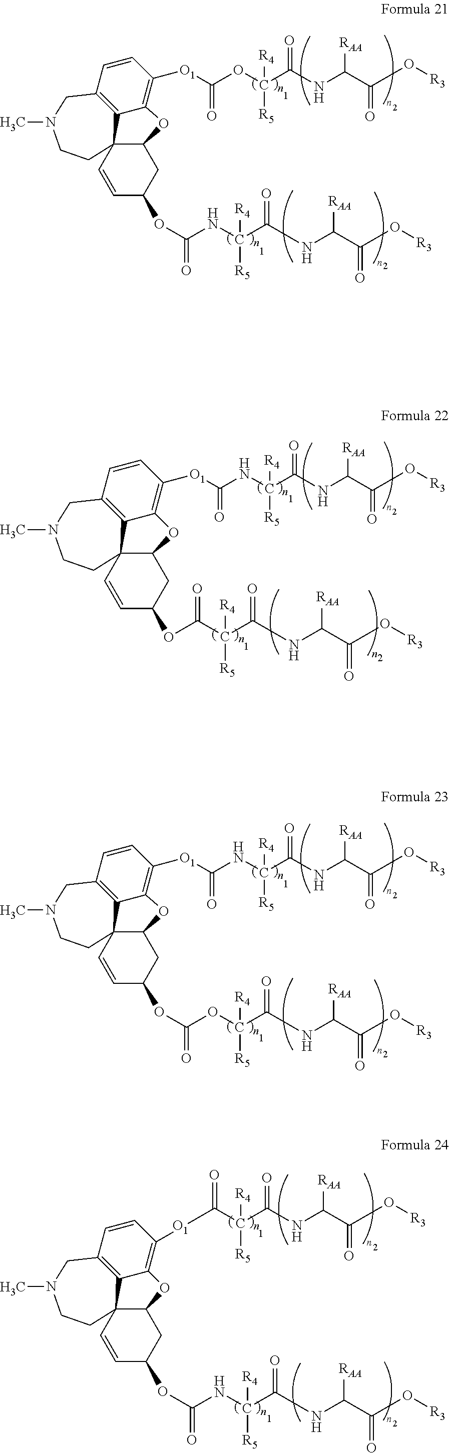

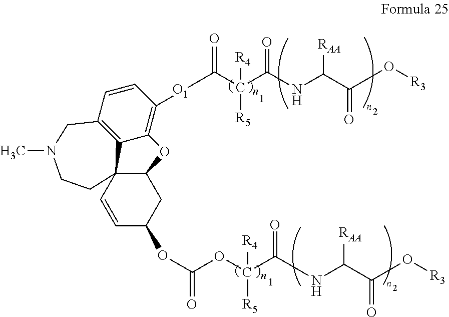

In one Formulae 17-25 embodiment, at least one occurrence of each occurrence of n₁ is independently 0, 1, 2, 3, or 4. In a further embodiment, at least one occurrence of R₃ is H and at least one occurrence of n₂ is independently 1, 2 or 3.

In one Formulae 17-25 embodiment, at least one occurrence of n₁ is independently 0, 1, 2 or 3. In a further embodiment, each occurrence of n₁ is independently 0, 1, 2 or 3 while each occurrence of R₃, R₄ and R₅ is hydrogen.

In one Formulae 17-25 embodiment, each occurrence of n₁ is independently 0, 1, 2 or 3, and each occurrence of n₂ is independently 1, 2 or 3 and R₃, R₄ and R₅ are each H. In a further embodiment, each occurrence of n₁ is 2.

In one Formulae 17-25 embodiment, each occurrence of n₁ is independently 0, 1, 2 or 3, each occurrence of n₂ is independently 1, 2 or 3 and each occurrence of R₃, R₄ and R₅ are H. In a further embodiment, each occurrence of n₁ is 2.

In one Formulae 17-25 embodiment, each occurrence of n₁ is independently 0, 1, 2 or 3, each occurrence of n₂ is independently 1, 2 or 3 and each occurrence of R₃, R₄ and R₅ is H. In a further embodiment, each occurrence of n₁ is 2, n₂ is 1.

In another Formulae 17-25 embodiment, each occurrence of n₁ is independently 1, 2 or 3 and each occurrence of n₂ is independently 1, 2 or 3. In a further embodiment, at least one occurrence of R₄ is

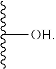

In yet another Formulae 17-25 embodiment, each occurrence of n₁ is independently 1 or 2 and each occurrence of n₂ is independently 1, 2, 3, 4 or 5. In a further embodiment, each occurrence of R_{AA} is independently a proteinogenic amino acid side chain.

In one Formulae 17-25 embodiment, each occurrence of n₁ is independently 0, 1 or 2, each occurrence of n₂ is independently 1 or 2 and each occurrence of R₃ is H. In a further embodiment, at least one occurrence of R₄ is

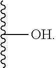

In another Formulae 17-25 embodiment, each occurrence of n₁ is independently 0, 1 or 2, each occurrence of n₂ is independently 1 or 2 and each occurrence of R₃ is H. In a further embodiment, at least one occurrence of R₄ is

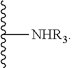

In a preferred Formulae 17-25 embodiment, the prodrug moiety of the present invention has one or two amino acids (i.e., each occurrence of n₂ is 1 or 2). In one embodiment, each occurrence of n₁ is independently 1 or 2 while each occurrence of n₂ is independently 1, 2 or 3.

In a preferred Formulae 17-25 embodiment, each occurrence of n₂ is independently 1,2 or 3 while each occurrence of R₃, R₄, and R₅ is H. In another embodiment, at least one occurrence of n₂ is 1. In yet another Formulae 17-25 embodiment, each occurrence of n₂ is 2. In yet another Formulae 17-25 embodiment, at least one occurrence of n₂ is 1 or 2 and each occurrence of R_{AA} is independently a proteinogenic amino acid side chain.

In another embodiment, the present invention is directed to a prodrug with two prodrug moieties—one dicarboxylic acid prodrug with at least one carbamate moiety, as provided in Formulae 26-34, shown below. For Formulae 26-34, each occurrence of R₃, R₄, R₅, R_{AA}, n₁ and n₂ is defined as provided for Formula 1.

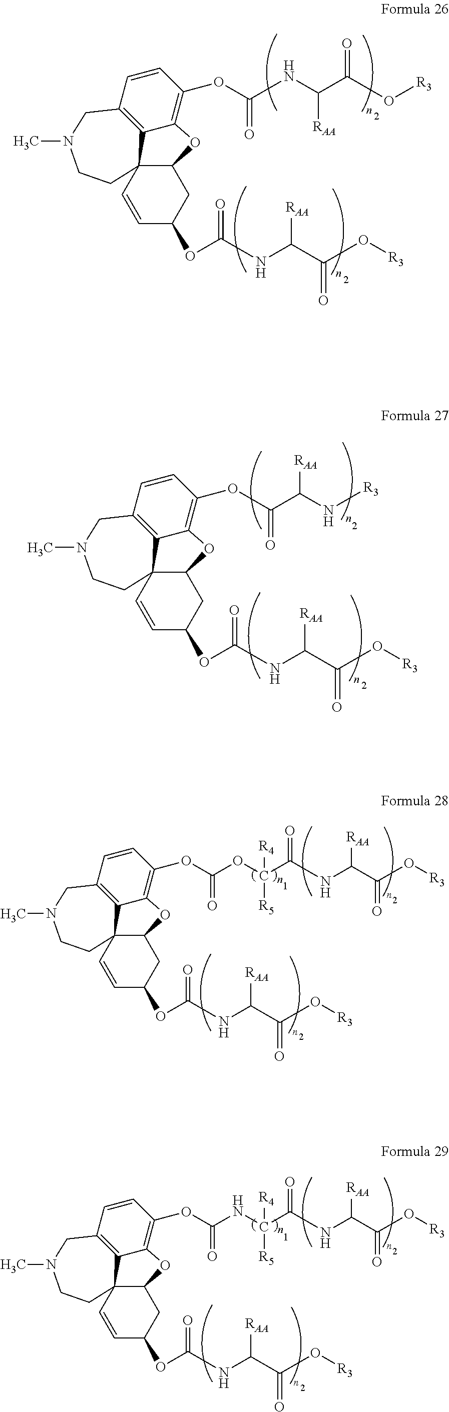

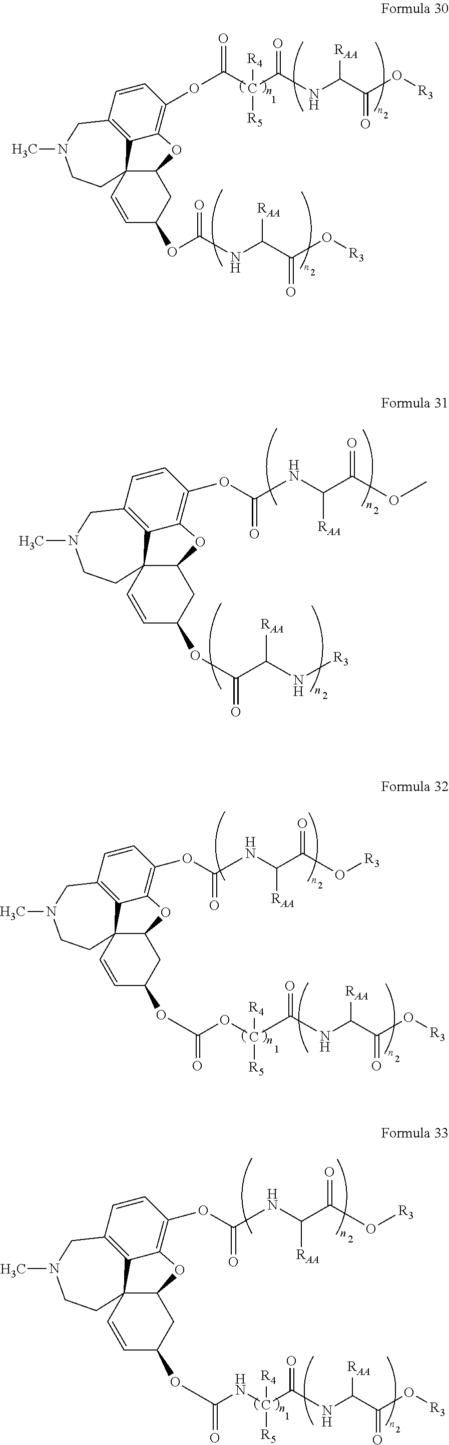

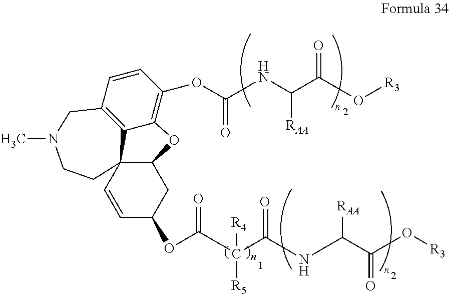

In yet another embodiment of the invention, prodrugs of Formulae 35-46 are provided as shown below. For Formulae 35-46, independently R_{AA}, R₃, R₄, R₅, R₆, R₇ X, X′, Y, Cy, n₁, n₃, n₄, and n₅ is defined as provided for Formula 1.

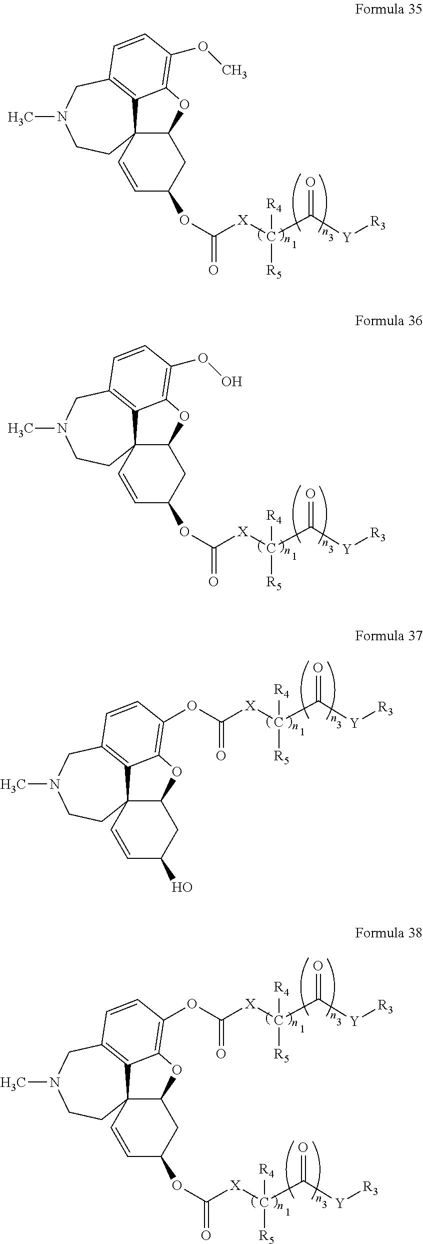

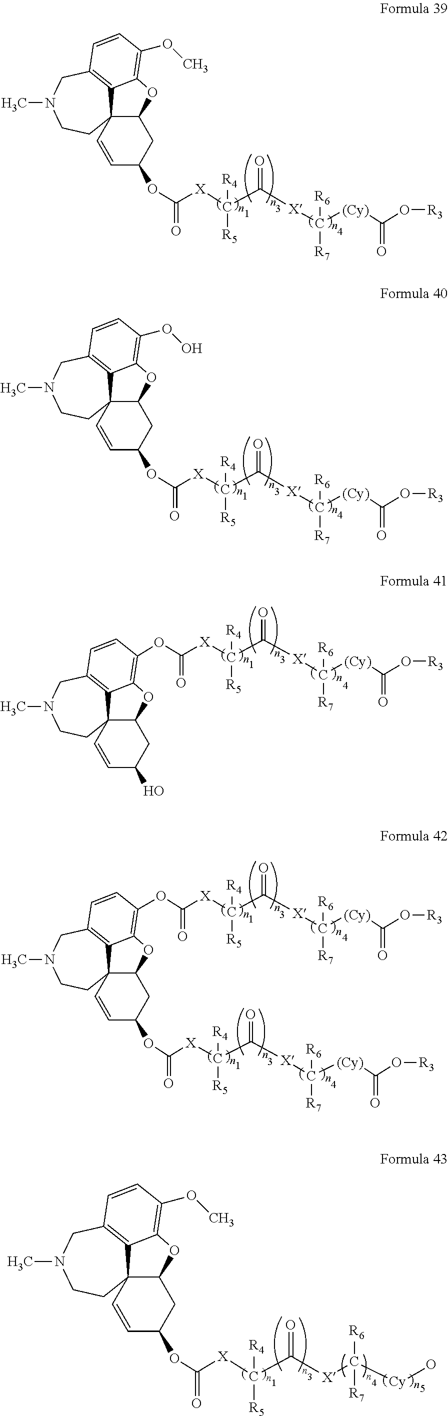

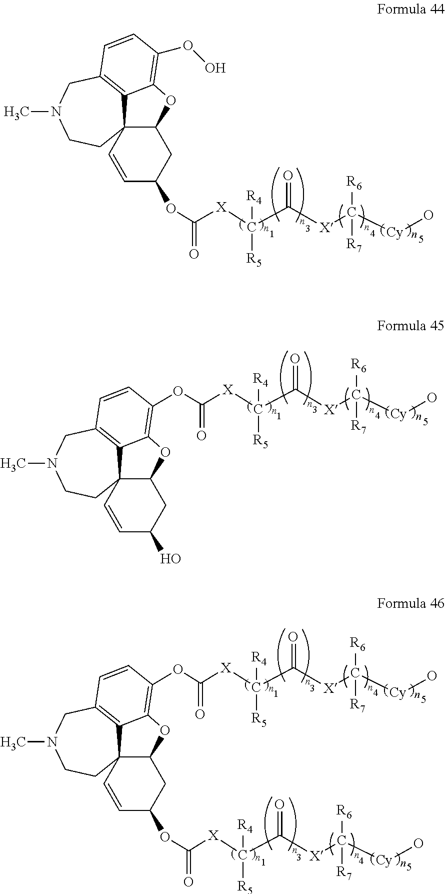

In one Formulae 35-46 embodiment, each occurrence of n₁ is independently 0, 1, 2, 3, or 4. In a further embodiment, each occurrence of R₃, R₄, R₅, R₆, and R₇ is H and each occurrence of n₃ is independently 0 or 1.

In another Formulae 35-46 embodiment, X and X′ is absent, each occurrence of n₁ is independently 0, 1, 2, 3, or 4. In a further embodiment, each occurrence of R₃, R₄, and R₅ is H and n₃ is 1 and n₄ and n₅ is 0.

In yet another Formulae 35-46 embodiment, X is absent, X′ is O, n₁ is independently 0, 1, 2, 3, or 4. In a further embodiment, each occurrence of R₃, R₄, and R₅ is H and n₃ is 1 and n₄ is 0 and Cy is aryl.

In another Formulae 35-46 embodiment, X and X′ is absent, each occurrence of n₁ is independently 0, 1, 2, 3, or 4. In a further embodiment, each occurrence of R₃, R₄, and R₅ is H and n₃ is 0 and n₄ is 0 and Cy is aryl.

In another Formulae 35-46 embodiment, X is absent, X′ is NH, each occurrence of n₁ is independently 0, 1, 2, 3, or 4. In a further embodiment, each occurrence of R₃, R₄, and R₅ is H and n₃ is 1 and n₄ is 0 and Cy is aryl.

In yet another Formulae 35-46 embodiment, X is absent, X′ is NH, each occurrence of n₁ is independently 0, 1, 2, 3, or 4. In a further embodiment, each occurrence of R₃, R₄, and R₅ is H and n₃ is 0 and n₄ is 0 and Cy is aryl.

In one Formulae 35-46 embodiment, X is absent, X′ is NH, each occurrence of n₁ is independently 0, 1, 2, 3, or 4. In a further embodiment, each occurrence of R₃, R₄, and R₅ is H and n₃ is 0 and n₄ is 0 and Cy is aryl.

In another Formulae 35-46 embodiment, X is absent, X′ is NH, each occurrence of n₁ is independently 0, 1, 2, 3, or 4. In a further embodiment, each occurrence of R₃, R₄, R₅, R₆, and R₇ is H and n₃ is 0 and n₄ is 1 and Cy is aryl.

In yet another Formulae 35-46 embodiment, X is absent, X′ is NH, each occurrence of n₁ is independently 0, 1, 2, 3, or 4. In a further embodiment, each occurrence of R₃, R₄, and R₅ is H and n₃ is 0 and n₄ is 0 and Cy is heteroaryl.

In yet another embodiment of the invention, prodrugs of Formulae 47 is provided as shown below.

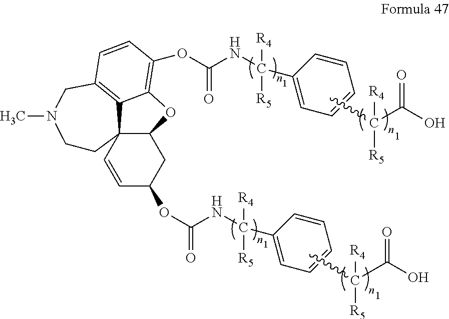

Each occurrence of R₄ and R₅ is independently selected from hydrogen,

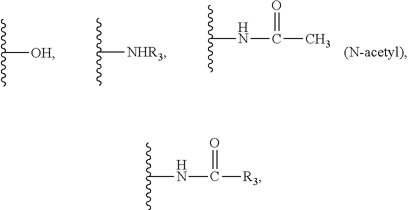

a substituted alkyl group, or an unsubstituted alkyl group;

In the case of a double bond in the carbon chain defined by n₁, R₄ is present and R₅ is absent on the carbons that form the double bond;

In Formula 47, each occurrence of n₁ can be independently 0, 1, 2, or 3.

In yet another embodiment of the invention, prodrugs of Formulae 48 is provided as shown below.

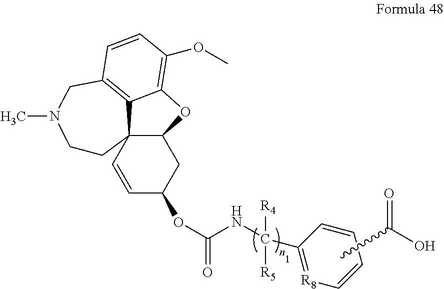

Each occurrence of R₄ and R₅ is independently selected from hydrogen,

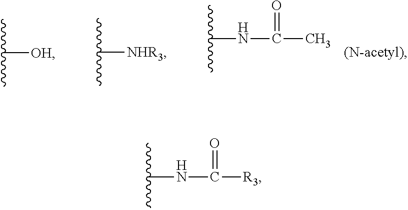

a substituted alkyl group, or an unsubstituted alkyl group;

R₈ is C or N;

In the case of a double bond in the carbon chain defined by n₁, R₄ is present and R₅ is absent on the carbons that form the double bond;

In Formula 48, each occurrence n₁ can be independently 0, 1, 2, or 3.

In yet another embodiment of the invention, prodrugs of Formulae 49 is provided as shown below.

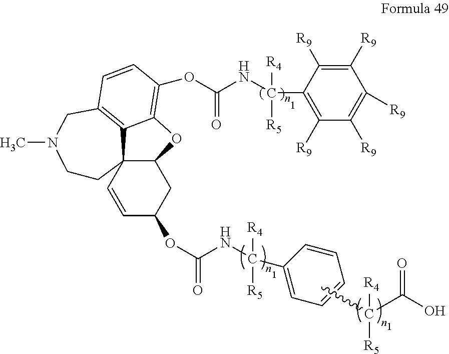

Each occurrence of R₄ and R₅ is independently selected from hydrogen,

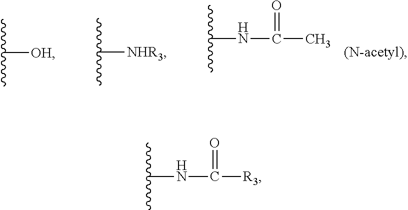

a substituted alkyl group, or an unsubstituted alkyl group;

Each occurrence of R₉ is independently hydrogen or

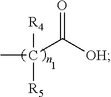

In the case of a double bond in the carbon chain defined by n₁, R₄ is present and R₅ is absent on the carbons that form the double bond;

In Formula 49, each occurrence of n₁ can be independently 0, 1, 2, or 3.

In yet another embodiment of the invention, prodrugs of Formulae 50 is provided as shown below.

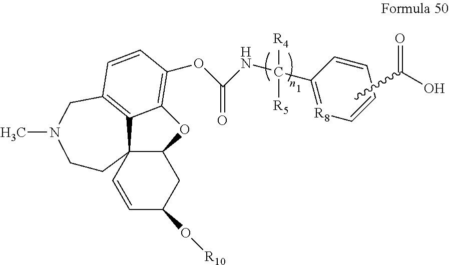

Each occurrence of R₄ and R₅ is independently selected from hydrogen,

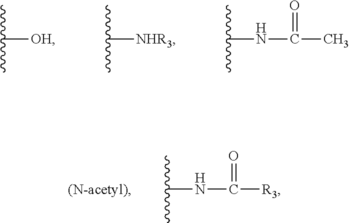

a substituted alkyl group, or an unsubstituted alkyl group;

Each occurrence of R₈ is independently C or N;

R₁₀ is hydrogen or

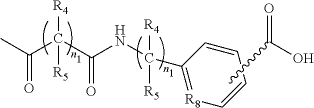

In the case of a double bond in the carbon chain defined by n₁, R₄ is present and R₅ is absent on the carbons that form the double bond;

In Formula 50, each occurrence of n₁ can be independently 0, 1, 2, or 3.

In yet another embodiment of the invention, prodrugs of Formulae 51, an example of a galantamine (dicarboxylic acid-PABA) ester is provided as shown below.

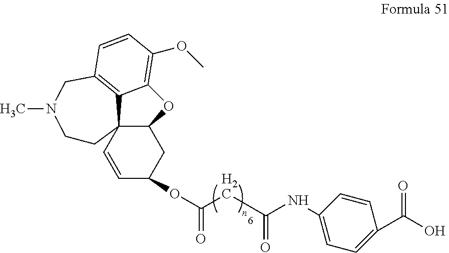

In Formula 51, n₆ is an integer from 0, 1, 2, 3, 4, 5, 6, 7, 8, 9, 10, 11, 12, 13, 14, 15, or 16.

In yet another embodiment of the invention, prodrugs of Formulae 52 is provided as shown below.

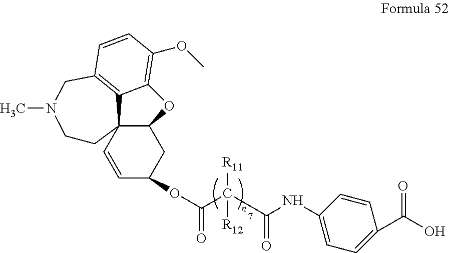

Each occurrence of R₁₁ and R₁₂ is independently selected from hydrogen,

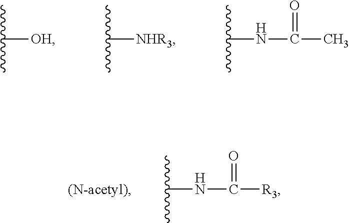

a substituted alkyl group, an unsubstituted alkyl group, a substituted aryl group, or an substituted aryl group;

R₁₁ and R₁₂ may be independently, geminal substituted or vincinal substituted;

In Formula 52, n₇ is an integer from 0, 1, 2, 3, 4, 5, 6, 7, 8, 9, 10, 11, 12, 13, 14, 15, or 16.

In yet another embodiment of the invention, prodrugs of Formulae 53 is provided as shown below.

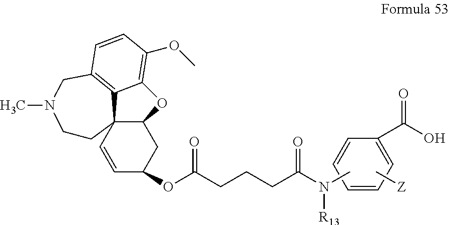

R₁₃ hydrogen, a substituted alkyl group, an unsubstituted alkyl group;

Z is hydrogen,

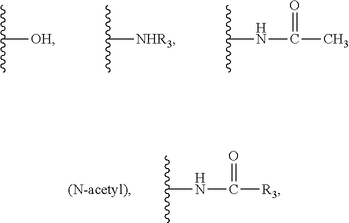

a substituted alkyl group, or an unsubstituted alkyl group;

R₁₁ and R₁₂ may be independently, geminal substituted or vincinal substituted;

In one embodiment, the phenolic function of galantamine's 3-OH metabolite may be linked to an amino acid or peptide by a simple ester linkage, or through a carbamate or dicarboxylic acid bridge such as a hemi-ester of, for example, malonic acid, succinic acid or glutaric acid or similar. Prodrugging the phenolic hydroxyl function serves specifically to ensure good oral bioavailability of the metabolite.

The prodrugs of the present invention are therefore likely to lead to improved patient compliance and greater predictability of pharmacologic response both within and between patients.

Although galantamine and 3-OH galantamine prodrugs represent two embodiments of the present invention that will offer the aforementioned advantages, these advantages are equally available to other acetylcholine esterase inhibitors or their active metabolites with derivatizable functions. Such compounds would include, but are not limited to, tacrine.

One embodiment of single amino acid simple ester of the parent drug would be with a valine residue.

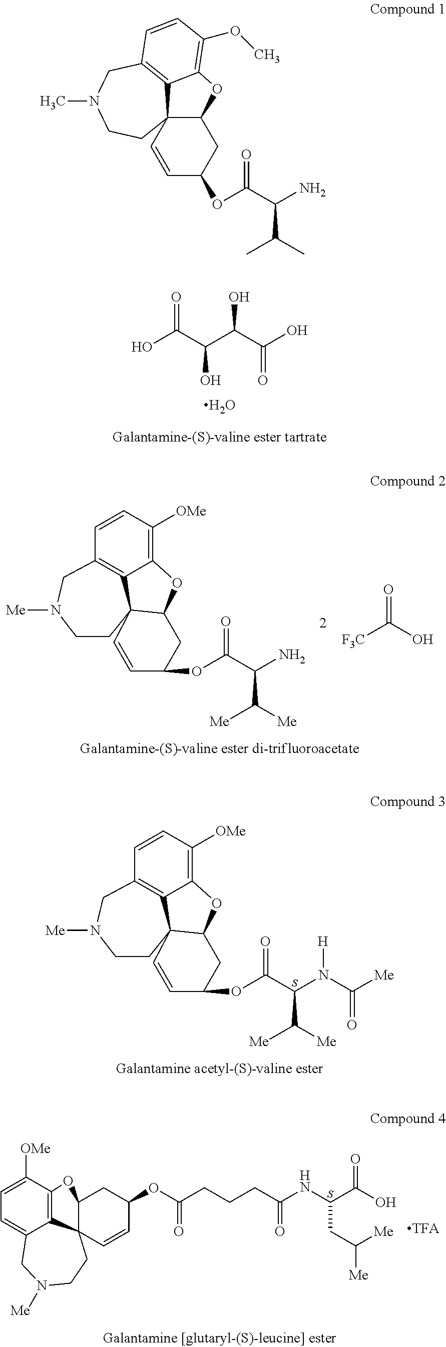

Other ester prodrug embodiments can include conjugates with isoleucine, phenylalanine and/or leucine.

In some embodiments, dipeptide conjugates of the simple esters of the parent drug include galantamine valine-valine ester, galantamine isoleucine-isoleucine ester and galantamine leucine-leucine ester.

In various embodiments, single amino acid carbamate conjugates of the parent drug include:

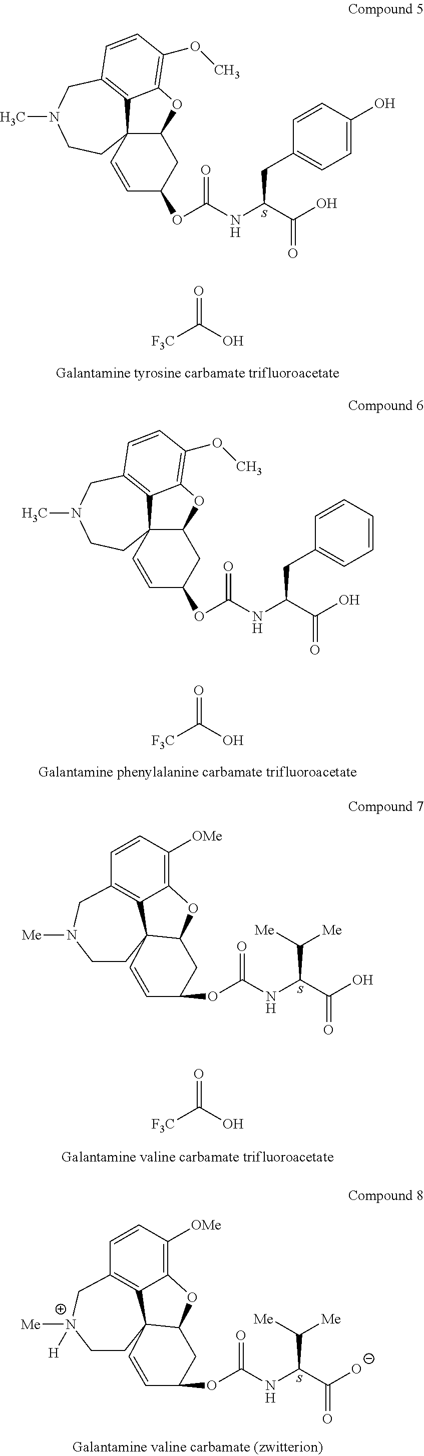

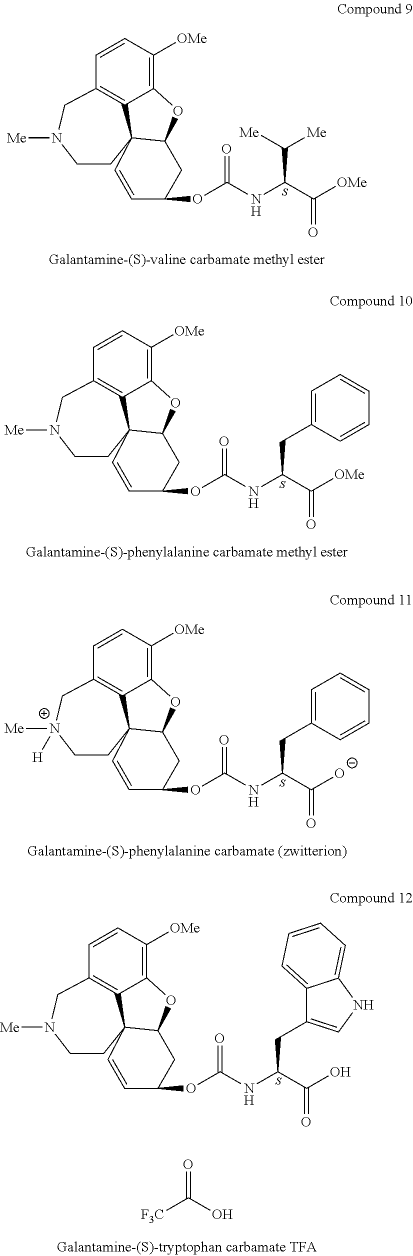

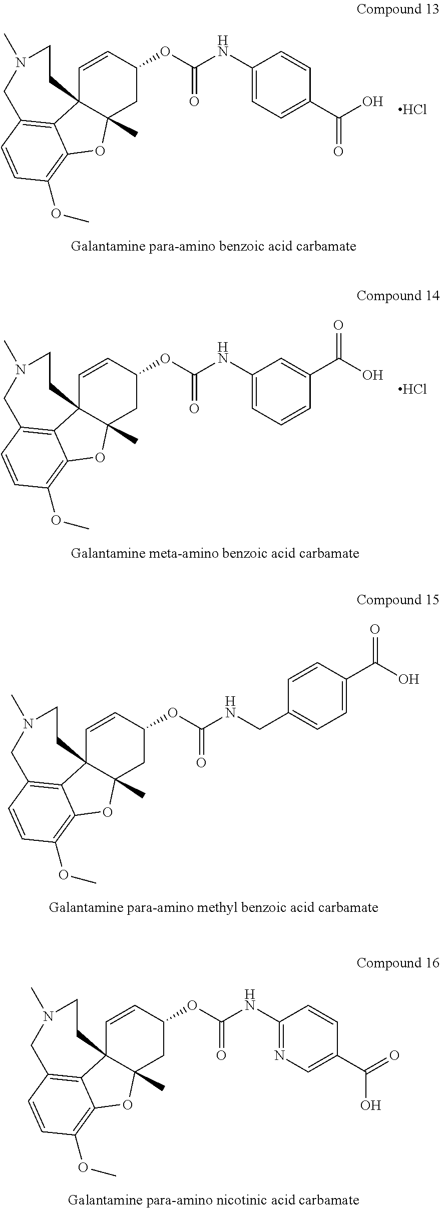

Some examples of galantamine dipeptide carbamate prodrugs include galantamine-tyrosine-tyrosine and galantamine-phenylalanine-phenylalanine.

Non-limiting examples of galantamine amino acid prodrugs that are succinyl linked include galantamine-valine (shown below), galantamine-isoleucine and galantamine-leucine.

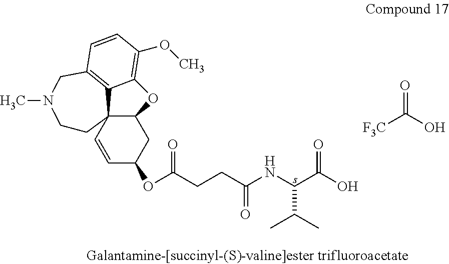

An intermediate metabolite of an amino acid or peptide prodrug of the present invention comprising a succinate bridge (e.g., Compound 17) is shown below as Compound 18. This intermediate metabolite, compound 18, can serve as a reservoir for the release of the active agent, wherein the succinate bridge is used to link a hydrolyzable amino acid or peptide to galantamine or galantamine metabolite. In otherwords, a galantamine prodrug of the present invention utilizing a succinate bridge can undergo metabolism to form a galantamine succinyl intermediate.

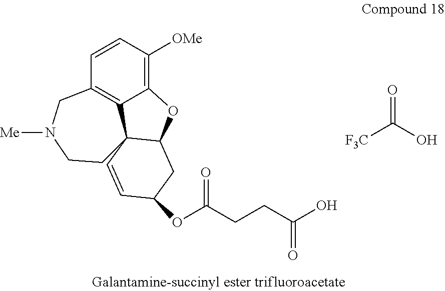

An intermediate metabolite of an amino acid or peptide prodrug of the present invention comprising a glutarate bridge is shown below as Compound 19. This intermediate metabolite, compound 19, can serve as a reservoir for the release of the active agent, wherein the glutarate bridge is used to link a hydrolyzable amino acid or peptide to galantamine or galantamine metabolite. In otherwords, a galantamine prodrug of the present invention utilizing a glutarate bridge can undergo metabolism to form a galantamine glutarate intermediate. Likewise, any galantamine prodrug of the present invention comprising a dicarboxylic bridge linker to a hydrolysable amino acid residue, can yield the related galantamine dicarboxylic intermediate.

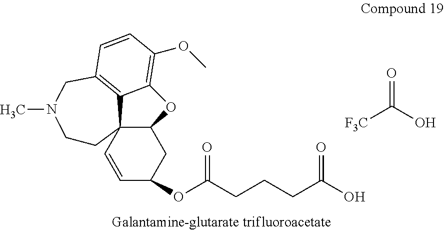

Dipeptide succinyl linked conjugates of galantamine include, but are not limited to galantamine succinyl valine-valine ester, galantamine succinyl isoleucine-isoleucine ester and galantamine succinyl leucine-leucine ester. Other dipeptide succinyl linked conjugates include, but are not limited to heteropeptides of leucine, isoleucine and valine.

Alternative dicarboxylic acid bridges to succinic acid (linking the drug to the amino acid) include, but are not limited to, malonic, glutaric and tartaric acids. Other dicarboxylic linkers for use with the present invention are given in tables 2 and 3. Additionally non proteinogenic amino acids such as para-amino benzoic as in galantamine glutaryl para-amino benzoic acid ester may be employed.

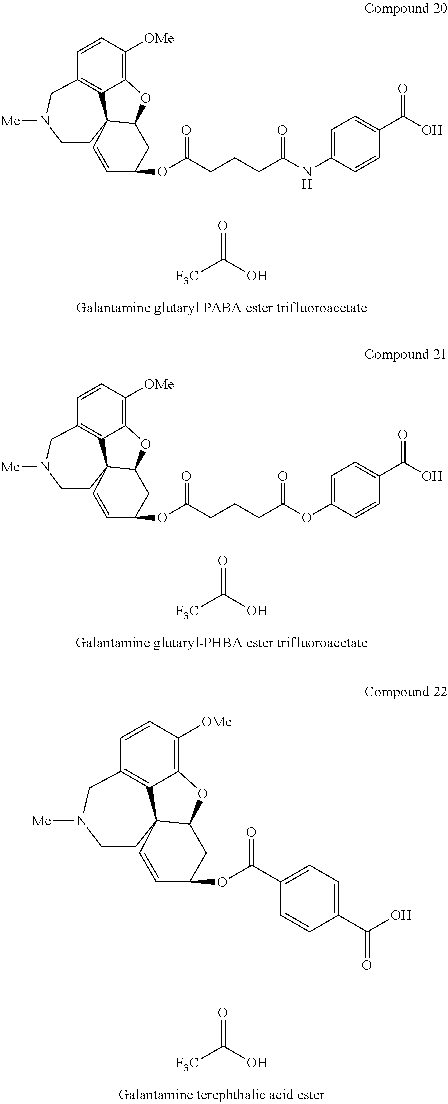

Amino acid conjugates of the active 3-OH metabolite can include those using either or both of the possible sites for derivatization, namely the 6 or 3 position. At either or both positions, single amino acids or short peptides can be conjugated either directly as simple esters or indirectly, through a carbamate or dicarboxylic acid linker.

In one embodiment, the pharmacologically active 3-OH galantamine prodrug is selected from the following:

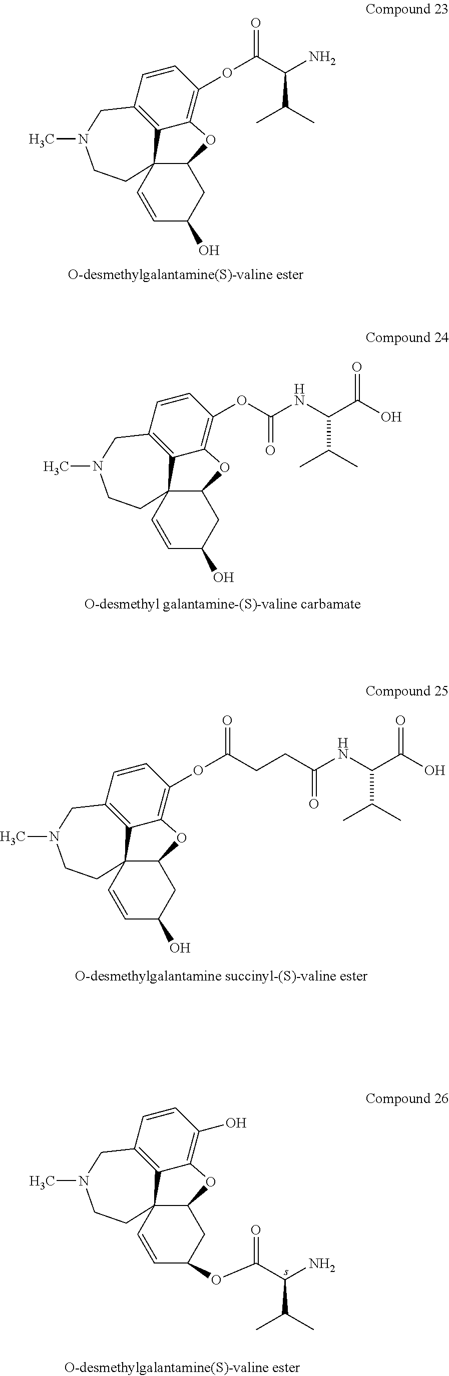

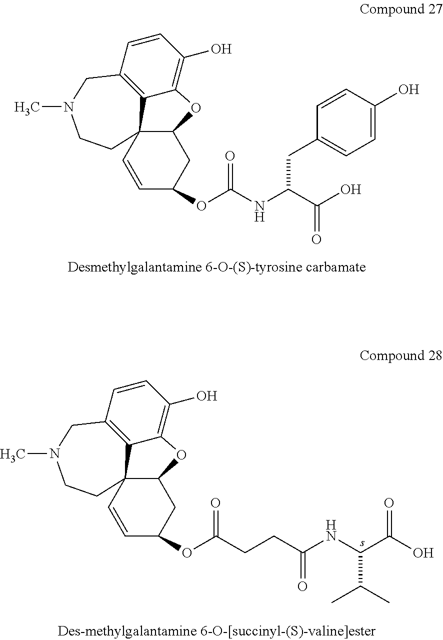

### Galantamine Prodrugs

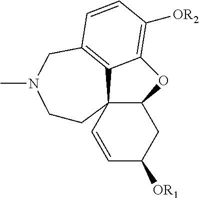

TABLE 4
Galantamine prodrugs

| Compd | Name | R₁ | R₂ |
|---|---|---|---|
| 29 | Galantamine [glutaryl-S)- valine] ester | 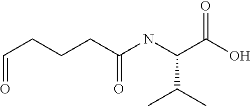 | Me |
| 30 | Galantamine [succinyl-S)- valine] ester | 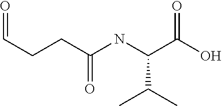 | Me |
| 31 | Galantamine [succinyl-S)- phenylalanine] ester | 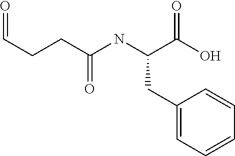 | Me |
| 32 | Galantamine [glutaryl-S)- phenylalanine] ester | 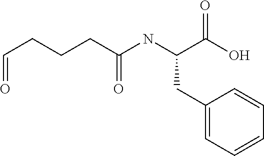 | Me |
| 33 | O-desmethyl galantamine 3- PABA carbamate | H | 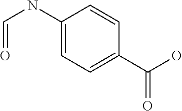 |
| 34 | O-desmethyl galantamine 3- glutaryl valine | H | 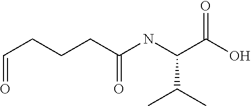 |
| 35 | O-desmethyl galantamine 3- succinyl valine | H | 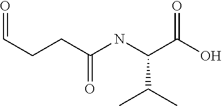 |
| 36 | O-desmethyl galantamine 3- valine carbamate | H | 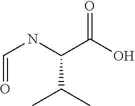 |
| 37 | Galantamine [glutaryl-S)- PABA] ester | 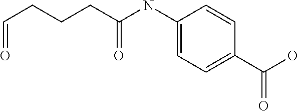 | Me |
| 38 | Galantamine [succinyl-S- PABA] ester | 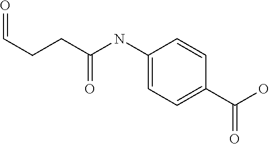 | Me |
| 39 | O-desmethyl galantamine [3- succinyl PABA] ester | H | 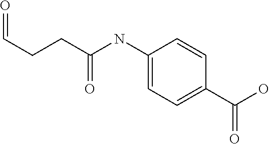 |
| 40 | O-desmethyl galantamine [3- glutaryl PABA] ester | H | 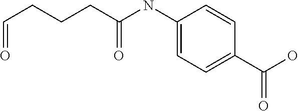 |
| 41 | Galantamine [glutaryl-S)- PHBA] ester | 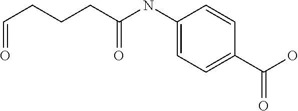 | Me |
| 42 | O-desmethyl galantamine [3- phenylalanine] carbamate | H | 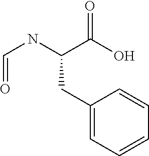 |
| 43 | O-desmethyl galantamine [3- PABA 6- PABA] carbamate | 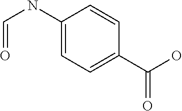 | 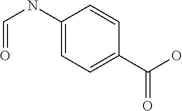 |
| 44 | O-desmethyl galantamine [3- sunccinyl PABA ester, 6- PABA carbamate] | 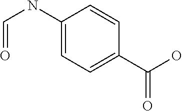 | 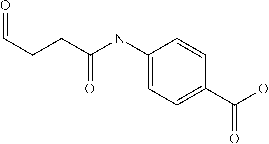 |
| 45 | O-desmethyl galantamine 3- [succinyl PABA] ester-6- [succinyl PABA] ester | 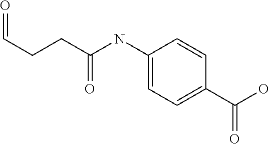 | 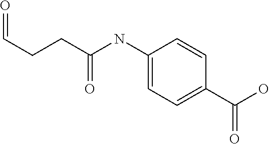 |
| 46 | O-desmethyl galantamine 3- [succinyl PABA] ester-6- [6- aminonicotinic acid] carbamate | 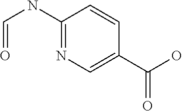 | 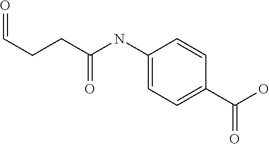 |
| 47 | O-desmethyl galantamine 3- [glutaryl PABA] ester-6-[6- aminonicotinic acid] carbamate | 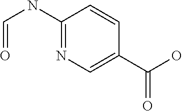 | 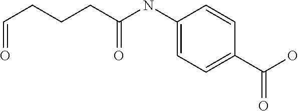 |
| 48 | O-desmethyl galantamine 3- [glutaryl PABA] ester-6-[glutaryl PABA] ester | 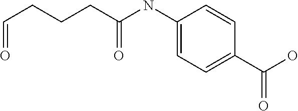 | 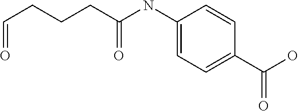 |
| 49 | O-desmethyl galantamine 3- [glutaryl PABA] ester-6-PABA carbamate | 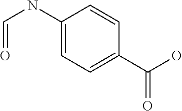 | 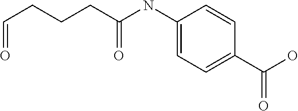 |
| 50 | O-desmethyl galantamine 3- [glutaryl 6- aminonicotinic acid] ester-6- [glutaryl 6- aminonicotinic acid] ester | 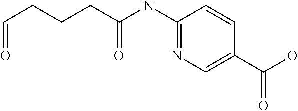 | 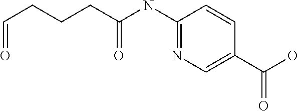 |
| 51 | O-desmethyl galantamine 3- [glutaryl 6- aminonicotinic acid] ester-6- PABA carbamate | 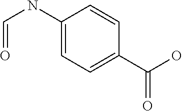 | 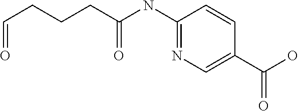 |
| 52 | O-desmethyl galantamine 3- [glutaryl 6- aminonicotinic acid] ester-6- [succinyl PABA] ester | 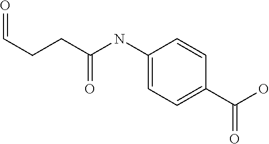 | 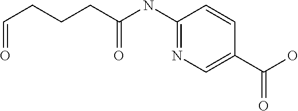 |
| 53 | O-desmethyl galantamine 3- [succinyl PABA] ester-6- [glutaryl 6- aminonicotinic acid] ester | 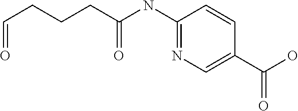 | 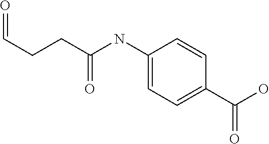 |
| 54 | O-desmethyl galantamine 3- [glutaryl PABA] ester-6- [succinyl PABA] ester | 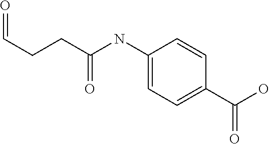 | 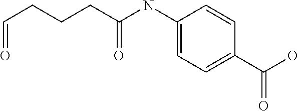 |
| 55 | O-desmethyl galantamine 3- [glutaryl PABA ester-6-(S)- valine carbamate | 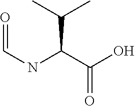 | 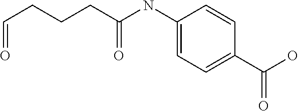 |
| 56 | O-desmethyl galantamine 3- [glutaryl PABA] ester-6-(S)- phenylalanine carbamate | 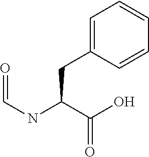 | 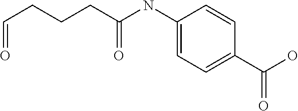 |
| 57 | O-desmethyl galantamine 3- [glutaryl PABA] ester-6-[(S)- phenylalanine methyl ester] carbamate | 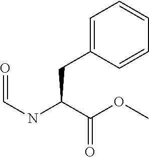 | 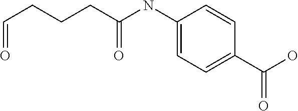 |
| 58 | O-desmethyl galantamine 3- [glutaryl PABA] ester-6-(S)- tyrosine carbamate | 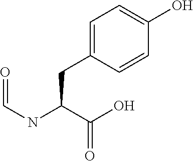 | 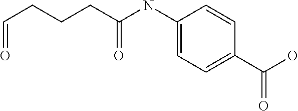 |
| 59 | O-desmethyl galantamine 3- [glutaryl PABA] ester-6-(S)- tryptophan carbamate | 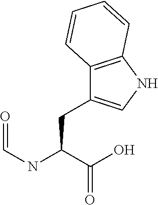 | 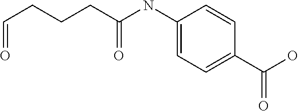 |
| 60 | O-desmethyl galantamine 3- PABA carbamate-6- [glutaryl PABA] ester | 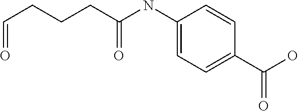 | 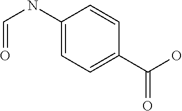 |
| 61 | O-desmethyl galantamine 3- (S)-valine carbamate-6- [glutaryl PABA] ester | 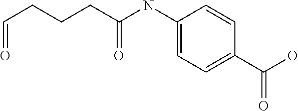 | 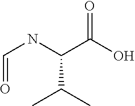 |
| 62 | O-desmethyl galantamine 3- PABA carbamate-6- [succinyl-(S)- phenylalanine] ester | 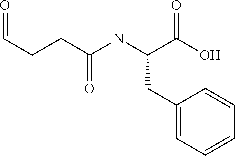 | 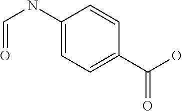 |
| 63 | O-desmethyl galantamine 3- (S)-valine carbamate-6- [succinyl-(S)- phenylalanine] ester | 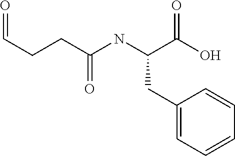 | 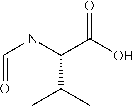 |
| 64 | O-desmethyl galantamine 3- [succinyl-(S)- phenylalanine] ester-6- [succinyl-(S)- phenylalanine] ester | 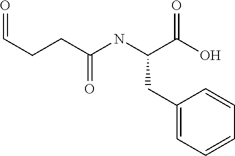 | 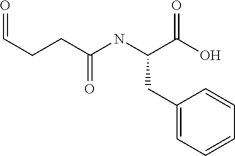 |
| 65 | O-desmethyl galantamine 3- [glutaryl PABA] ester-6- [succinyl-(S)- phenylalanine] ester | 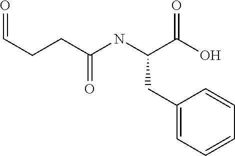 | 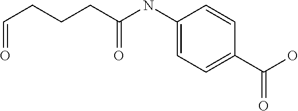 |
| 66 | O-desmethyl galantamine 3- (S)-valine carbamate-6- [glutaryl PABA] ester | 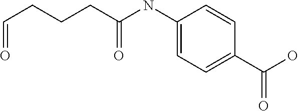 | 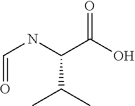 |
| 67 | O-desmethyl galantamine 3- [succinyl-(S)- phenylalanine] ester-6- [succinyl PABA] ester | 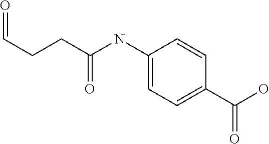 | 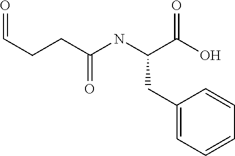 |
| 68 | O-desmethyl galantamine 3- PABA carbamate-6- [succinyl PABA] ester | 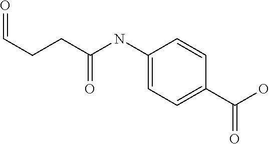 | 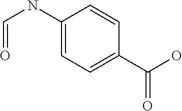 |
| 69 | O-desmethyl galantamine 3- PABA carbamate-6- [glutaryl PABA] ester | 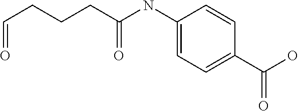 | 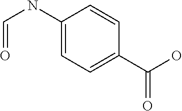 |
| 70 | O-desmethyl galantamine 3- PABA carbamate-6- [glutaryl PHBA] ester | 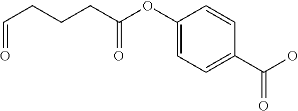 | 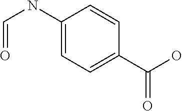 |
| 71 | O-desmethyl galantamine 3- (S)-valine carbamate-6- [glutaryl PHBA] ester | 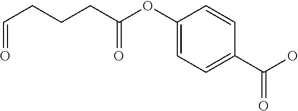 | 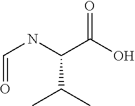 |

### Advantages of Compounds of the Invention

Without wishing to be bound by any particular theory, emesis associated with galantamine may be mediated by a direct local action within the gastrointestinal (GI) tract. Such effects are believed to result largely from a direct cholinergic action on the gut following oral ingestion of galantamine, with a prior study showing a direct action of galantamine on isolated gastrointestinal smooth muscle (Turiiski et al. (2004). Eur. J. Pharmacol. 13, 233-239). Additional evidence for a direct local effect of galantamine came from a study by Leonard, in which oral and intranasal doses of galantamine were compared with respect to their emetic potential in a ferret model (Leonard et al. (2007). Int J. Pharmaceutics 335, 138-146). Despite the attainment of much higher systemic levels of the drug after intranasal dosing, the incidence of emesis was much greater following oral dosing with galantamine.

Local concentrations of galantamine within the stomach following a typical 24 mg dose (˜200-400 μM) substantially exceed the IC₅₀ for inhibition of acetylcholine esterase (0.35 μM). Thus, acetylcholine esterase secreted in the gut will be inhibited leading to a local elevation of acetylcholine and the consequential cholinomimetic effects on the gut. Further evidence for local effects of galantamine within the GI tract comes from the observation that transdermally delivered galantamine and rivastigmine (another AChEI) are associated with a reduced incidence of emesis (U.S. Patent Publication No. 2007/0104771 and Yang et al. Drug (2007). CNS 21, 957-965).

A transiently inactivated galantamine prodrug may represent an alternative means of minimizing the drug's direct effect on the gut. Such a prodrug may preclude direct contact of the active drug with the gut and should therefore lessen the potential to cause nausea, emesis and other adverse GI effects. Subsequent to oral absorption of the prodrug, and cleavage of the prodrug moiety, galantamine would be available for systemic action.

Without wishing to be bound to any particular theory, it is believed that the amino acid or peptide portion of galantamine and/or 3-OH-hydroxy galantamine prodrugs may exploit the inherent di- and tripeptide transporter Pept 1 within the digestive tract to effect absorption. Alternatively other transporters may be involved such as the fluoroscein/nateglinide when the conjugating moiety is an aromatic carboxylic acid such as para amino benzoic acid. Once absorbed these preferred prodrugs are subject to hydrolysis releasing the active drug into the systemic circulation. Avoidance of direct contact between active drug and gut wall minimizes the risk of emesis while the assisted absorption of the prodrug by Pept 1 ensures more consistent plasma drug levels. In the case of prodrugs of 3-hydroxy galantamine, such compounds avoid the usual polymorphically expressed CYP2D6 clearance mechanism of galantamine leading to more reproducible plasma levels across the whole patient population. Furthermore, prodrugs of either the drug or its active metabolite also have the potential to sustain plasma concentrations as the result of the continuing generation of the active principal from its inactivated form.

### Uses of the Compounds of the Invention

In one embodiment of the invention, a method is provided for treating a disorder in a subject in need thereof with galantamine. The method comprises orally administering a therapeutically effective amount of a galantamine prodrug or a pharmaceutically acceptable salt thereof to a subject in need thereof, wherein the galantamine prodrug is comprised of galantamine or its 3-OH metabolite covalently bonded to an amino acid or peptide of 2-9 amino acids in length. The disorder may be one treatable with galantamine. For example the disorder may be a memory or cognition disorder (e.g., Alzheimer's Disease, vascular dementia, Parkinson's Disease, Huntington's Disease, infection-induced dementia). In a further embodiment, the galantamine prodrug has a second prodrug moiety.

In one embodiment, a method for improving memory and/or cognitive function in a subject in need thereof is provided. The method comprises orally administering a therapeutically effective amount of a galantamine prodrug or a pharmaceutically acceptable salt thereof to a subject in need thereof, wherein the galantamine prodrug is comprised of galantamine or its 3-OH metabolite covalently bonded to an amino acid or peptide of 2-9 amino acids in length. In a further embodiment, the galantamine prodrug has a second prodrug moiety.

In another embodiment of the invention, the galantamine prodrugs provided herein confer the benefit of reducing adverse GI side effects, including nausea and vomiting, associated with oral ingestion of the parent compound. The method comprises orally administering a therapeutically effective amount of a galantamine prodrug or a pharmaceutically acceptable salt thereof, or a composition thereof, to a subject in need thereof, wherein the galantamine prodrug is comprised of galantamine or its 3-OH metabolite covalently bonded to an amino acid or peptide of 2-9 amino acids in length, and wherein upon oral administration, the prodrug or pharmaceutically acceptable salt minimizes, if not completely avoids, the gastrointestinal side effects usually seen after oral administration of the unbound galantamine. In a further embodiment, the galantamine prodrug of the present invention has two prodrug moieties.

In yet another embodiment of the invention, the amino acid and peptide prodrugs of the present invention improve galantamine's overall pharmacokinetic profile and consistency of achievement of therapeutic plasma concentrations, as compared to the administration of galantamine itself.

In a further embodiment, a method for sustaining plasma drug concentrations and hence reducing dosing frequency and consequently improving patient compliance is provided. Sustaining or maintaining plasma drug concentrations may result in fewer daily administrations of the galantamine prodrug, thus limiting the daily exposure of the GI tract to galantamine or the galantamine prodrug. Less daily exposure of the GI tract to galantamine or the galantamine prodrug may result in fewer GI side effects with reduced emesis and diarrhea and more consistent drug availability ensuring less unintentional drug loss and thus greater consistency in blood levels This should lead to improvements in patient compliance. The sustainment or maintenance of blood levels is an important feature or attribute of the galantamine prodrugs of the present invention, which allows the prolonged generation, conversion, or release of the galantamine, or an active metabolite of the galantamine, or an active metabolite of a galantamine prodrug from a prodrug reservoir. The active form is released into the blood to achieve sustained plasma levels of the galantamine or active metabolite. T_{>50% Cmax}, the time or period for which the plasma drug concentration remains at or above 50% of the maximum concentration, is a useful measurement of sustainment or maintenance of blood levels.

The reservoir from which the active form of the drug is released comprises both the whole prodrug or an intermediate metabolite (e.g., Compounds 18 and 19). The proportion of prodrug to intermediate metabolite will vary on the identity of the particular prodrug.

Without being bound by theory, it is believed that present invention may include the formation of a prodrug metabolite prior to the formation of the parent drug upon administration to a patient. The prodrug metabolite may accumulate so as to form a reservoir in the bloodstream. The prodrug metabolite may then further metabolize to form the parent molecule at a specific rate related to the disappearance of the parent compound. The reservoir in the bloodstream of the patient may allow a T_{>50% Cmax} that is larger than that obtained with the an equivalent dose of the parent drug, allowing the constant generation of the parent drug as required by the patient. In an embodiment of the present invention the increase in T_{>50% Cmax} is equal to or greater than 100% of that obtained with the administration of an equivalent dose of the parent drug. In another embodiment of the present invention the T_{>50% Cmax} is between about 100% and about 300% of that obtained with the administration of an equivalent dose of the parent drug.

In still another embodiment, a method for reducing inter- or intra-subject variability of galantamine serum levels is provided. The method comprises administering to a subject, or group of subjects, in need thereof, a therapeutically effective amount of a galantamine prodrug of the present invention (e.g., a prodrug of Formula 1), a pharmaceutically acceptable salt thereof, or a composition thereof, wherein the galantamine prodrug is comprised of galantamine or its 3-OH metabolite covalently bonded to an amino acid or peptide of 2-9 amino acids in length. The disorder may be one treatable with galantamine.

### Salts and Solvates Derivatives of the Compounds of the Invention

The methods of the present invention further encompass the use of salts, or solvates, of the prodrugs of galantamine/3-OH galantamine described herein, for example salts of the prodrugs of Formulae 1-53 given above. In various embodiments, the invention disclosed herein is meant to encompass all pharmaceutically acceptable salts of galantamine/3-OH galantamine prodrugs, and specifically, all pharmaceutically acceptable salts of the compounds of Formulae 1-53.

Typically, a pharmaceutically acceptable salt of a prodrug of galantamine used in the practice of the present invention is prepared by reaction of the prodrug with a desired acid as appropriate. This could alternatively involve making a salt of the free phenolic function or carboxylic function in the case of carbamate and dicarboxylic acid bridged ester prodrugs. The salt may precipitate from solution and be collected by filtration or may be recovered by evaporation of the solvent. For example, an aqueous solution of an acid such as hydrochloric acid may be added to an aqueous suspension of the prodrug and the resulting mixture evaporated to dryness (lyophilized) to obtain the acid addition salt as a solid. Alternatively, the prodrug may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the prodrugs may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts of those prodrugs containing an acidic function (carboxylic acid or phenol) may be formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine and N-methylglucamine.

The base addition salts of the acidic compounds are prepared by contacting the free acid form with a sufficient amount of the desired base to produce the salt in the conventional manner. The free acid form may be regenerated by contacting the salt form with an acid and isolating the free acid.

Compounds useful in the practice of the present invention of the 3-OH metabolite may have both a basic and an acidic center and may therefore be in the form of zwitterions.

Salts of the basic azepine nitrogen would include, but not limited to, a range of differing lipophilicities e.g TFA, HBr, HCl, tartrate, maleate, tosylate, (toluene sulphonic acid) camsylate (camphor sulphonic acid), and napsylate (naphthalene sulphonic acid).

Those skilled in the art of organic chemistry will appreciate that many organic compounds can form complexes, i.e., solvates, with solvents in which they are reacted or from which they are precipitated or crystallized, e.g., hydrates with water. The salts of compounds useful in the present invention may form solvates such as hydrates useful therein. Techniques for the preparation of solvates are well known in the art (see, e.g., Brittain, Polymorphism in Pharmaceutical solids. Marcel Decker, New York, 1999.). The compounds useful in the practice of the present invention can have one or more chiral centers and, depending on the nature of individual substituents, they can also have geometrical isomers.

### Pharmaceutical Compositions of the Invention

While it is possible that, for use in the methods of the invention, the prodrug may be administered as the bulk substance, it is preferable to present the active ingredient in a pharmaceutical formulation, e.g., wherein the agent is in admixture with a pharmaceutically acceptable carrier selected with regard to the intended route of administration and standard pharmaceutical practice.

The formulations of the invention may be immediate-release dosage forms, i.e., dosage forms that release the prodrug at the site of absorption immediately, or controlled-release dosage forms, i.e., dosage forms that release the prodrug over a predetermined period of time. Controlled release dosage forms may be of any conventional type, e.g., in the form of reservoir or matrix-type diffusion-controlled dosage forms; matrix, encapsulated or enteric-coated dissolution-controlled dosage forms; or osmotic dosage forms. Dosage forms of such types are disclosed, for example, in Remington, The Science and Practice of Pharmacy, 20^{th} Edition, 2000, pp. 858-914. The formulations of the present invention can be administered from one to six times daily, depending on the dosage form and dosage.

Absorption of amino acid and peptide prodrugs of galantamine/3-OH-galantamine is likely to proceed via an active transporter such as Pept 1. This transporter is believed to be largely confined to the upper GI tract and as such may restrict the utility of conventional sustained release formulations for continued absorption along the whole length of the GI tract. For those prodrugs of galantamine/3-OH galantamine which do not result in sustained plasma drugs levels due to continuous systemic generation of active from a plasma “reservoir” of prodrug, a gastroretentive or mucoretentive formulation analogous to those used in metformin products such as Glumetz® metformin or Gluphage XR® metformin may be useful. The former exploits a drug delivery system known as Gelshield Diffusion™ Technology while the latter uses a so-called Acuform™ delivery system. In both cases, the concept is to slow drug delivery into the ileum maximizing the period over which absorption take place and effectively prolonging plasma drug levels. Other drug delivery systems affording delayed progression along the GI tract may also be of value.

For those galantamine/3-OH galantamine prodrugs that do not require the sophistication of the aforementioned delivery systems conventional formulations as described below should be adequate.

Alternatively other transporters may be involved such as the fluoroscein/nateglinide when the conjugating moiety is an aromatic carboxylic acid such as para amino benzoic acid.

In one embodiment, the present invention provides a pharmaceutical composition comprising at least one active pharmaceutical ingredient (i.e., a prodrug of galantamine or 3-OH galantamine), or a pharmaceutically acceptable derivative (e.g., a salt or solvate) thereof, and a pharmaceutically acceptable carrier. In particular, the invention provides a pharmaceutical composition comprising a therapeutically effective amount of at least one prodrug of the present invention, or a pharmaceutically acceptable derivative thereof, and a pharmaceutically acceptable carrier.

For the methods of the invention, the prodrug employed in the present invention may be used in combination with other therapies and/or active agents. Accordingly, the present invention provides, in a further aspect, a pharmaceutical composition comprising at least one compound useful in the practice of the present invention, or a pharmaceutically acceptable salt or solvate thereof, a second active agent, and, optionally a pharmaceutically acceptable carrier.

When combined in the same formulation it will be appreciated that the two compounds must be stable and compatible with each other and the other components of the formulation. When formulated separately they may be provided in any convenient formulation, conveniently in such manner as are known for such compounds in the art.

The prodrugs used herein may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit, 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and flavoring agents may be provided in the pharmaceutical composition. Examples of preservatives include sodium benzoate, ascorbic acid and esters of p-hydroxybenzoic acid. Antioxidants and suspending agents may also be used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, for example see International Patent Application No. WO 02/00196 (SmithKline Beecham).

The compounds and pharmaceutical compositions of the present invention are intended to be administered orally (e.g., as a tablet, sachet, capsule, pastille, pill, bolus, powder, paste, granules, bullets or premix preparation, ovule, elixir, solution, suspension, dispersion, gel, syrup or as an ingestible solution). In addition, compounds may be present as a dry powder for constitution with water or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

The compounds and pharmaceutical compositions of the present invention can be administered orally in a water or aqueous solution-based formulation. In other embodiments, the compounds and pharmaceutical compositions of the present invention can be administered orally in an oil-based formulation. One possible advantage of an oil-based formulation is to preserve the prodrug's integrity particularly while resident in the GI tract.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions useful herein include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g, banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers useful herein include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants useful herein include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyanisole.

The pharmaceutical compositions of the invention may contain from 0.01 to 99% weight per volume of the prodrugs encompassed by the present invention.

### Dosages

Appropriate patients to be treated according to the methods of the invention include any human or animal in need of such treatment. Methods for the diagnosis and clinical evaluation of Alzheimer's disease, are well known in the art. Thus, it is within the skill of the ordinary practitioner in the art (e.g, a medical doctor or veterinarian) to determine if a patient is in need of treatment. The patient is preferably a mammal, more preferably a human, but can be any animal, including a laboratory animal in the context of a clinical trial or screening or activity experiment employing an animal model. Thus, as can be readily appreciated by one of ordinary skill in the art, the methods and compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, research animals, such as mice, rats, rabbits, goats, sheep, pigs, dogs, cats, etc., avian species, such as chickens, turkeys, songbirds, etc.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

In one embodiment, an effective daily amount of a prodrug of galantamine (expressed as galantamine free base) is from 1 mg to 1000 mg, preferably from 1 mg to 100 mg. For example, the prodrugs encompassed by the present invention may be formulated in a dosage form that contains from about 20 mg to about 80 mg of the prodrug per unit dose. In a preferred embodiment, an effective daily amount of the prodrugs of galantamine is from 40 to 80 mg. 1, 5, 10, 20, 30, 40, 50, 60, 70, 80, 90, or 100 mg of the prodrug per unit dose. In another embodiment, the dosage form contains from 15, 25, 75, 125, 150, 175, 200, 250, 300, 350, 400, 450, 500, 550, 600, 650, 700, 750, 800, 850, 900, 950, or 1000 mg of the prodrug per unit dose.

In one embodiment, an effective daily amount of a prodrug of active metabolite, expressed as 3-OH galantamine free base, 3-OH galantamine is from 1 mg to 300 mg, preferably from 1 mg to 30 mg. For example, the prodrugs encompassed by the present invention may be formulated in a dosage form that contains from about 5 mg to about 30 mg of the prodrug per unit dose. Another example, the prodrugs encompassed by the present invention may be formulated in a dosage form that contains from about 10, 20, 25, 30, 40, 50, 60, 70, 75, 80, 90, 100, 125, 150, 175, 200, 225, 250, 275, or 300 mg of the prodrug per unit dose. In a preferred embodiment, an effective amount of the prodrugs of formulae 1-53 is from about 5 to about 15 mg

Depending on the severity of cognitive impairment to be treated, a suitable therapeutically effective and safe dosage, as may be determined within the skill of the art, and without undue experimentation, maybe administered to subjects. For oral administration to humans, the daily dosage level of the prodrug may be in single or divided doses. The duration of treatment may be determined by one of ordinary skill in the art, and should reflect the nature of the condition and/or the rate and degree of therapeutic response to the treatment.

In the methods of treating the condition the prodrugs encompassed by the present invention may be administered in conjunction with other therapies and/or in combination with other active agents. For example, the prodrugs encompassed by the present invention may be administered to a patient in combination with other active agents used in the management of Alzheimer's disease. In such combination therapies the prodrugs encompassed by the present invention may be administered prior to, concurrent with, or subsequent to the other therapy and/or active agent.

Where the prodrugs encompassed by the present invention are administered in conjunction with another active agent, the individual components of such combinations may be administered either sequentially or simultaneously in separate or combined pharmaceutical formulations by any convenient route. When administration is sequential, either the prodrugs encompassed by the present invention or the second active agent may be administered first. For example, in the case of a combination therapy with another active agent, the prodrugs encompassed by the present invention may be administered in a sequential manner in a regimen that will provide beneficial effects of the drug combination. When administration is simultaneous, the combination may be administered either in the same or different pharmaceutical compositions. For example, the prodrugs encompassed by the present invention and another active agent may be administered in a substantially simultaneous manner, such as in a single capsule or tablet having a fixed ratio of these agents or in multiple, separate capsules or tablets for each agent.

When the prodrugs encompassed by the present invention are used in combination with another agent active in the methods for treating pain, the dose of each compound may differ from that when the compound is used alone. Appropriate doses will be readily appreciated by those skilled in the art.

### EXAMPLES

The present invention is further illustrated by reference to the following Examples. However, it should be noted that these Examples, like the embodiments described above, are illustrative and are not to be construed as restricting the enabled scope of the invention in any way.

General Synthesis Procedures

An activated amino acid or peptide, such as BOC-(S)-valine, can be added to galantamine or 3-OH galantamine, in the presence of DCC and DMAP. After a chromatography step, the galantamine prodrug can be deprotected with trifluoroacetic acid. A salt of the prodrug can then be formed, for example, by adding a solution of tartaric acid in methanol to the prodrug.

Examples 1-6 demonstrate the general scheme of covalently attaching galantamine to a variety of chemical moieties resulting in different embodiments of the present invention. From this disclosure, one of skill in the art would be able to synthesize further embodiments of the present invention using standard organic chemical synthesis reactions as described herein.

### Example 1

### Synthesis of Galantamine-(S)-Valine Ester Tartrate

The synthesis of galantamine-(S)-valine ester tartrate was carried as shown in Scheme 1.

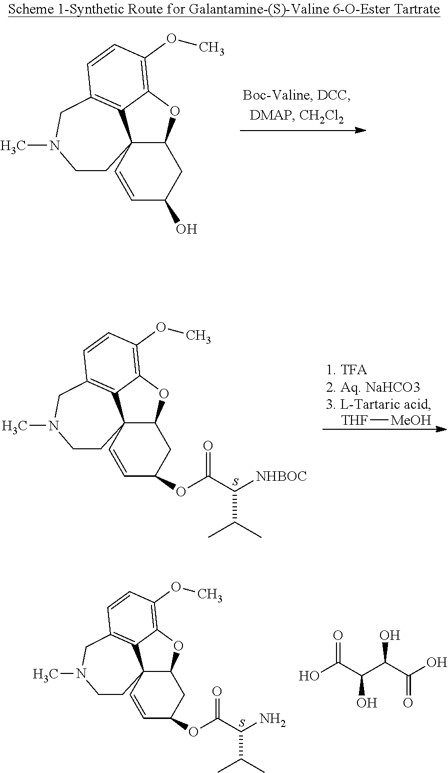

Galantamine was coupled with BOC-(S)-valine, in the presence of dicyclohexylcarbodi-imide (DCC) in dichloromethane, and the reaction was catalyzed by N,N-dimethylaminopyridine (DMAP). The reaction gave an 89% yield of the ester in very good purity after chromatography. TFA deprotection with a very short reaction time of just 5 minutes afforded galantamine-(S)-valine ester ditrifluoroacetate, which was neutralized by extraction from aqueous sodium bicarbonate into dichloromethane.

The resulting diamine free base was dissolved in tetrahydrofuran and treated with a solution of L-tartaric acid in methanol. The required compound crystallized immediately and was collected by filtration, washed, and dried under vacuum. HPLC analysis indicted 96% purity and CHN analysis showed the product was a monohydrate.

### ¹H NMR (DMSO-d₆) spectrum

6.72 (d, J=8.1 Hz, 1H, ArH), 6.58 (d, J=8.1 Hz, 1H, ArH), 6.42 (d, J=10.5 Hz, 1H, alkene H), 5.80 (quartet, J=5.1 Hz, 1H, alkene H), 5.29 (broad s, 1H, CH—O.CO), 4.51 (broad s, 1H, valine α-CH), 4.17+3.64 (AB system, J=14.7 Hz, ArCH₂N), 3.98 (s, 2H, 2×tartrate CH), 3.72 (s, 3H, ArOCH₃), 3.45 (m, 1H, CH—O—Ar), 3.29 (m, 1H, 0.5×CH₂N), 2.98 (m, 1H, 0.5×CH₂N), 2.5−2.0 (m, 4H, 1.5×CH₂+valine β-CH), 2.30 (m, 3H, NCH₃), 1.56 (d, 1H, J=13.2 Hz, 0.5×CH₂), 0.92 (t, J=7.7 Hz, 6H, 2×valine CH₃).

### Example 2

### Synthesis of Galantamine-(S)-Valine Ester Trifluoroacetate

The synthesis of galantamine-(S)-valine ester trifluoroacetate was carried as shown in Scheme 1.

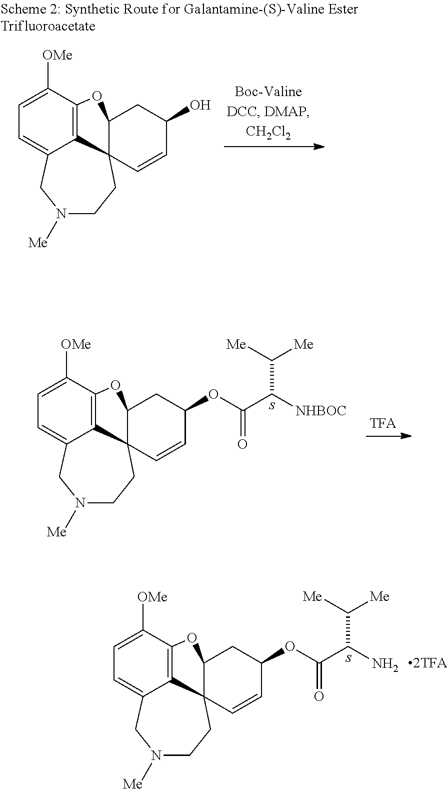

### ¹H NMR (DMSO-d₆) spectrum

8.33 (broad s, 3H, NH₃⁺), 6.89 (d, J=8.1 Hz, 1H, ArH), 6.81 (d, J=8.1 Hz, 1H, ArH), 6.52 (m, 1H, alkene H), 5.90 (m, 1H, alkene H), 5.38 (broad s, 1H, CH—O.CO), 4.9−4.2 (m, 4H, CH—O—Ar+valine α-CH+ArCH₂N), 3.78 (s, 3H, ArOCH₃), 3.00 (broad s, 2H, CH₂N), 2.6−2.0 (m, 8H, 2×CH₂+NCH₃+valine β-CH), 1.00 (m, 6H, 2×valine CH₃).

### Example 3

### Synthesis of Galantamine-(S)-Phenylalanine Carbamate Trifluoroacetate

This synthetic route is shown in the Scheme 3 below.

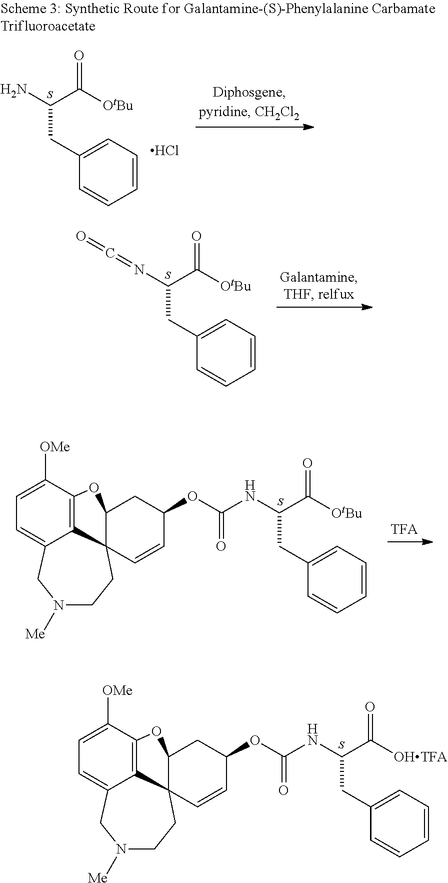

(S)-Phenylalanine tert-butyl ester hydrochloride was treated with diphosgene in dichloromethane in the presence of pyridine. After stirring for 2 hours with warming from 0° C. to room temperature, the required isocyanate was isolated after aqueous work-up and was used immediately in the next reaction step.

Reaction of the isocyanate with galantamine free-base in refluxing tetrahydrofuran for 2 days afforded, after column chromatography, a good yield of galantamine-(S)-phenylalanine carbamate tert-butyl ester, in the form of its free base.

The free base was stirred in trifluoroacetic acid (TFA) for 30 minutes to cleave the tert-butyl ester. This reduced reaction time was introduced to help minimise the formation of possible by-products. Evaporation of the trifluoroacetic acid followed by azeotroping with chloroform afforded the desired galantamine-(S)-phenylalanine carbamate trifluoroacetate in nearly quantitative yield, as a glassy solid.

### ¹H NMR (DMSO-d₆) spectrum

7.53 (d, J=8.1 Hz, 1H, carbamate NH), 7.4−7.2 (m, 5H, 5×phenylalanine ArH), 6.88 (d, J=8.1 Hz, 1H, ArH), 6.81 (d, J=8.1 Hz, 1H, ArH), 6.31 (m, 1H, alkene H), 5.86 (m, 1H, alkene H), 5.02 (broad, 1H, CH—O.CO), 4.9−4.0 (m, 4H, CH—O—Ar+phenylalanine α-CH+ArCH₂N), 3.77 (s, 3H, ArOCH₃), 3.60 (m, 1H, 0.5×CH₂N), 3.1−2.8 (m, 3H, 0.5×CH₂N+phenylalanine β-CH₂), 2.4−2.0 (m, 7H, 2×CH₂+NCH₃).

### Example 4

### Synthesis of Galantamine-(S)-Tyrosine Carbamate Trifluoroacetate

The synthetic route to galantamine-(S)-tyrosine carbamate trifluoroacetate is outlined in Scheme 3. Di-t-butyl protected (S)-tyrosine, commercially available, was used as the starting material.

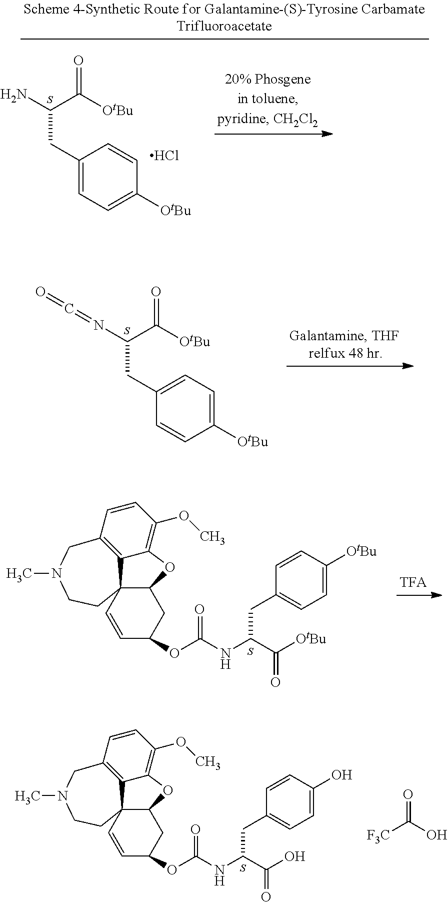

H-Tyr(O^{t}Bu)—O^{t}Bu hydrochloride was treated with 20% phosgene in toluene solution in dichloromethane in the presence of pyridine to convert it to the isocyanate. After stirring for 2 hours with warming from 0° C. to room temperature, the required isocyanate was isolated after aqueous work-up and was used immediately in the next reaction step.

Galantamine free base was reacted with the isocyanate in refluxing tetrahydrofuran for 2 days to afford, after column chromatography, a good yield of the doubly-protected carbamate, in the free base form.

Deprotection using trifluoroacteic acid (90 minutes at room temperature) removed both protecting groups. After concentration followed by trituration with diethyl ether, galantamine-(S)-tyrosine carbamate trifluoroacetate was obtained as a hygroscopic, glassy solid with >95% purity as analyzed by LCMS and NMR.

### ¹H NMR (DMSO-d₆) spectrum

7.53 (d, J=6.9 Hz, 1H, carbamate NH), 7.05 (d, J=8.1 Hz, 2H, 2×tyrosine ArH), 6.88 (d, J=8.4 Hz, 1H, ArH), 6.81 (d, J=8.4 Hz, 1H, ArH), 6.69 (d, J=8.1 Hz, 2H, 2×tyrosine ArH), 6.33 (m, 1H, alkene H), 5.86 (m, 1H, alkene H), 5.03 (broad, 1H, CH—O.CO), 4.9-4.0 (m, 4H, CH—O—Ar+tyrosine α-CH+ArCH₂N), 3.78 (s, 3H, ArOCH₃), ca. 3.6 (obscured m, 1 H, 0.5×CH₂N), 3.1−2.7 (m, 3H, 0.5×CH₂N+tyrosine β-CH₂), 2.4−2.0 (m, 7H, 2×CH₂+NCH₃).

### Example 5

### Synthesis of Galantamine-[Succinyl-(S)-Valine]Ester TFA Salt

The synthesis of galantamine-[succinyl-(S)-valine]ester trifluoroacetate is outlined in Scheme 5.

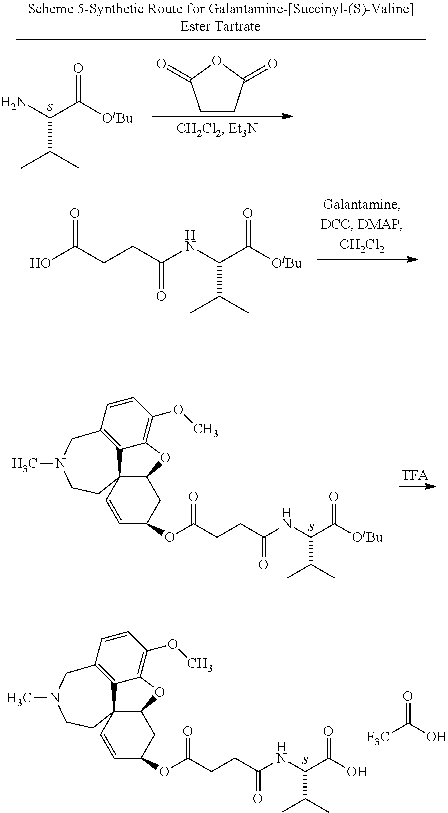

The necessary succinyl-valine half amide was synthesized according to a literature method (Stupp et al. (2003). J. Am. Chem. Soc., 125, 12680-12681) by reacting (S)-valine tert-butyl ester hydrochloride with succinic anhydride in dichloromethane in the presence of triethylamine. After an aqueous work-up, the product was isolated by crystallization from a mixture of diethyl ether and petrol, as a fluffy white powder.

Coupling of galantamine with this material mediated by dicyclohexylcarbodi-imide (DCC) in dichloromethane catalyzed by N,N-dimethylaminopyridine (DMAP) gave a high yield (81%) of the half-ester in good purity after chromatography. Deprotection of the valine carboxyl group using trifluoroacetic acid, followed by trituration with diethyl ether afforded galantamine-[succinyl-(S)-valine]ester trifluoroacetate in quantitative yield, as a white powder.

### ¹H NMR (DMSO-d₆) spectrum

7.99 (d, J=8.4 Hz, 1H, amide NH), 6.88 (d, J=8.4 Hz, 1H, ArH), 6.80 (d, J=8.4 Hz, 1H, ArH), 6.41 (m, 1H, alkene H), 5.88 (m, 1H, alkene H), 5.22 (broad, 1H, CH—O.CO), 4.9−4.2 (m, 3H, CH—O—Ar+ArCH₂N), 4.14 (m, 1H, valine α-CH), 3.78 (s, 3H, ArOCH₃), 3.6−2.3 (m, 2H, CH₂N), 2.97 (s, 3H, CH₃N), 2.6−2.1 (m, 8H, 2×galantamine CH₂+2×succinyl CH₂), 2.04 (m, 1H, valine. β-CH₂), 0.86 (d, J=7.5 Hz, 2×valine CH₃).

### Example 6

### Synthesis of Galantamine (Glutaryl-PABA) Ester Trifluoroacetate

The initial synthesis of galantamine glutarate ester trifluoroacetate, the key intermediate for the preparation of galantamine glutarate-linked prodrugs, was accomplished in three steps (shown in Scheme 6 below).

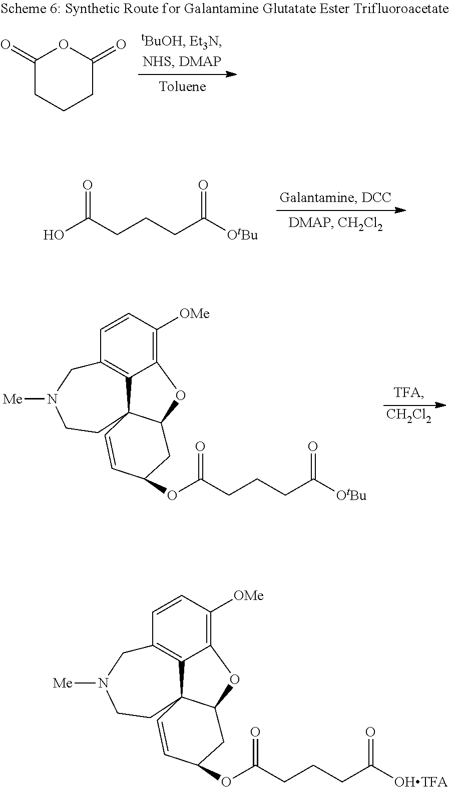

Glutaric anhydride was ring-opened with tert-butanol in toluene in the presence of triethylamine, N-hydroxysuccinimide (NHS) and DMAP to afford mono tert-butyl glutarate. This was coupled to galantamine using DCC in the presence of DMAP in dichloromethane to afford the tert-butyl protected galantamine glutarate ester, which was purified by column chromatography. Removal of the tert-butyl ester in trifluoroacetic acid and dichloromethane proceeded smoothly to give galantamine glutarate trifluoroacetate in good yield.

Coupling of galantamine glutarate trifluoroacetate with 4-aminobenzoic acid (PABA) was accomplished in two steps, as shown in Scheme 7 below:

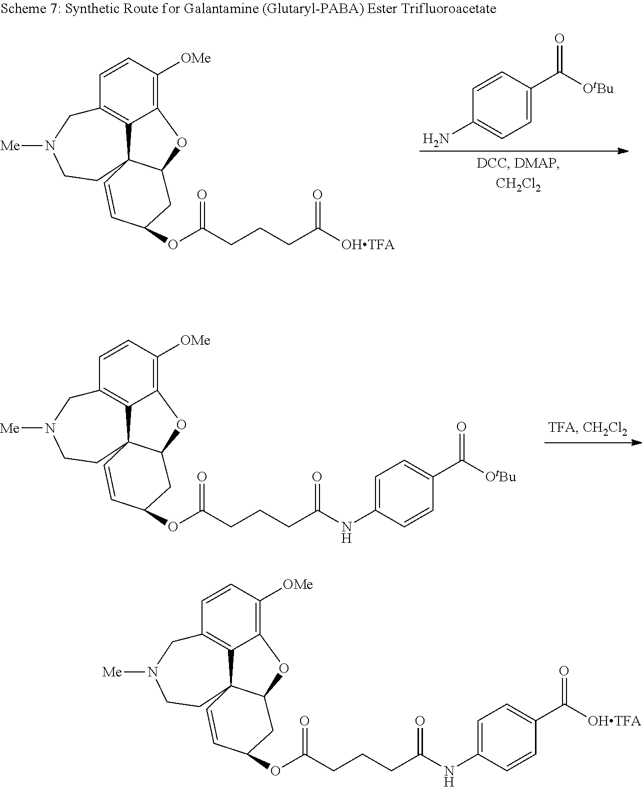

Galantamine glutarate trifluoroacetate was coupled to tert-butyl-4-aminobenzoate using DCC in dichloromethane to give the corresponding tert-butyl protected galantamine (glutaryl-PABA) ester, which was purified by column chromatography.

Removal of the tert-butyl ester in TFA and dichloromethane gave the corresponding trifluoroacetate salt of galantamine (glutaryl-PABA) ester trifluoroacetate which did not require any further purification.

¹H NMR (DMSO-d₆) spectrum: 10.80 and 9.95 (br s, 1H, NH⁺), 10.21 (s, 1H, CONH), 7.87 (d, J=8.8 Hz, 2H, 2×PABA ArH), 7.69 (d, J=8.8 Hz, 2H, 2×PABA ArH), 6.85 (d, J=8.3 Hz, 1H, ArH), 6.78 (d, J=8.3 Hz, 1H, ArH), 6.47−6.37 (m, 1H, alkene H), 5.95−5.86 (m, 1H, alkene H), 5.24 (broad, 1H, CH—O.CO), 4.87−4.59 (m, 2H, ArCH₂N), 4.39−4.19 (m, 1H, CH—O—Ar), 3.86−3.74 (m, 1H, 0.5×CH₂N), 3.72 (s, 3H, ArOCH₃), 3.61−3.48 (m, 1H, 0.5×CH₂N), 2.98 (d, J=4.2 Hz, 1.5H, 0.5×CH₃N), 2.57 (br, 1H, 0.33×CH₃N), 2.43 (s, 0.5H, 0.17×CH₃N), 2.41−2.19 (m, 6H, 2×COCH₂ and galantamine CH₂), 2.12−1.99 (m, 1.5H, 0.75×galantamine CH₂), 1.87−1.77 (m, 2.5H, 0.25×galantamine CH₂ and glutaryl CH₂).

### Example 7

### Assessment of the Chemical Stability of Various Galantamine Prodrugs in Simulated Gastric and Intestinal Fluid

In order to avoid the potential for directly mediated local interactions with the stomach and gut mucosa, a prodrug may remain intact during its residency in the gut lumen prior to its absorption. To evaluate the stability of potential prodrugs these compounds were incubated in USP simulated gastric and intestinal juice at 37° C. for 2 h or in some cases the more biorelevant Fasted State Simulated Intestinal Fluid (FaSSIF) or Fed State Simulated Intestinal Fluid (FeSSIF). See www.dissolutiontech.com/DTresour/200405Articles/DT200405_A03.pdf

Methodology

Aqueous solutions of various galantamine prodrugs were prepared in USP stimulated gastric pH 1.2 and intestinal juice pH 6.8 and incubated for 1 or 2h respectively at 37° C. In later studies the methodology was refined to use more representative intestinal juice designated FaSSIF (fasted) and FeSSIF (fed). Incubate aliquots were removed for HPLC analysis of both prodrug and active drug.

Results

These are shown in Table 5 and reveal that these prodrugs are essentially stable in either simulated USP gastric juice or USP simulated gastric juice or FaSSIF/FeSSIF—thus, providing encouragement that no direct local action of the drug on the stomach or within the small intestine may occur using these prodrugs. This would be expected to reduce the possibility of any locally mediated emetic response.

TABLE 5
Stability of various galantamine prodrugs under likely physiological conditions

| | USP SGF pH 1.2 | USP SGF pH 1.2 | USP SIF pH 6.8 | USP SIF pH 6.8 |
|---|---|---|---|---|
| | 37 deg. C. 0 hours | 37 deg. C. 1 hour | 37 deg. C. 0 hours | 37 deg. C. 2 hours |
| Compound | % Prodrug Remaining | % Prodrug Remaining | % Prodrug Remaining | % Prodrug Remaining |
| Galantamine | 94.7% | 94.7% | 95.9% | 95.6% |
| phenylalanine | | | | |
| carbamate | | | | |
| Galantamine | 100-% | 100% | 100% | 100% |
| PABA Carbamate | | | | |
| Galantamine | 100% | 100% | 100% | 100% |
| MABA | | | | |
| Carbamate | | | | |
| Galantamine | 100% | 100% | 100% | 100% |
| Benzyl ABA | | | | |
| Carbamate | | | | |
| Galantamine | 100% | 100% | 100% | 100% |
| Para Amino | | | | |
| Nicotinate | | | | |
| Carbamate | | | | |
| | FaSSIF (pH 6.5) | FaSSIF (pH 6.5) | FeSSIF (pH 6.5) | FeSSIF(pH 6.5) |
| | 37 deg. C. 0 hours | 37 deg. C. 2 hour | 37 deg. C. 0 hours | 37 deg. C. 2 hours |
| Compound | % Prodrug Remaining | % Prodrug Remaining | % Prodrug Remaining | % Prodrug Remaining |
| Galantamine- | 103% | 95% | 82% | 91% |
| [succinyl-(S)-Val] | | | | |
| Ester | | | | |
| Galantamine- | 96% | 86% | 85% | 90% |
| (glutaryl-PABA) | | | | |
| Carbamate | | | | |

### Example 8

### Comparative Bioavailability of Galantamine from Various Prodrugs in the Dog and Monkey

For prospective prodrugs to be of value it is essential that firstly the prodrug is efficiently absorbed from the GI tract and secondly that the parent active drug molecule is regenerated once the prodrug is in the systemic circulation. A comparative oral bioavailability study was therefore carried out on a number of prospective prodrugs in two higher species namely dogs and monkeys.

Test substances (i.e., galantamine and various prodrug conjugates,) were administered by oral gavage to various groups of dogs or monkeys. Blood samples were taken at various times after dosing and submitted to analysis for the parent drug using a validated LC-MS-MS assay.

Pharmacokinetic parameters derived from the plasma analytical data, including t½, AUC, absolute bioavailability, etc., were determined using the program Win Nonlin®.

Results

Results are shown in Table 6 & 7.

The results of this study show a wide range in bioavailability of galantamine from the various amino acid conjugates. The largest collection of prodrug conjugates was investigated in the dog, with a smaller cohort examined in the monkey.

While the highest systemic availability in the dog was seen with the simple valine ester (see Table 6), the longest sustainment of plasma drug concentrations was seen after administration of the succinyl valine ester and the glutaryl PABA ester prodrugs, the T_{>50% Cmax} values (the time plasma levels remained at or above 50% of Cmax) being 6.75±1.08 h and 4.05±0.98 h respectively as compared to 2.3±0.38 h following administration of the unconjugated galantamine. Both prodrugs gave good overall systemic availability, being 58.9 and 56% respectively. In addition to these two dicarboxylate bridged ester prodrugs, two carbamate bridged amino acid (the phenylalanine and tryptophan conjugates) displayed good pharmacokinetics. In the monkey (see Table 7), again the best performing prodrug conjugates were the succinyl valine ester and the glutaryl PABA ester with relative bioavailabilities of 39 and 20%, respectively. The periods of sustainment of plasma drug levels were >5.0 h and 5.26±0.69 h respectively compared to 1.66±0.39 h following administration of the unconjugated galantamine.

TABLE 6
Comparative pharmacokinetic parameters for galantamine in the dog following
oral administration of 1 mg equivalent galantamine free base/kg of galantamine
itself or various amino acid prodrugs of galantamine

| | Cₘₐₓ | AUC | Relative oral |
|---|---|---|---|
| Compound Name | (mean ng/mL ± sd) | (mean ng · h/mL ± sd) | Bioavailability (%) |
| Galantamine HBr | 216 ± 63 | 453 ± 75 | — |
| Galantamine-(S)-valine ester | 171 ± 58 | 539 ± 211 | 119.0 |
| Galantamine-[succinyl-(S)- | 44.0 ± 6.4 | 267 ± 52 | 58.9 |
| valine] ester | | | |
| Galantamine [glutaryl-PABA] | 71 ± 23 | 326 ± 26 | 56.0 |
| ester | | | |
| Galantamine-(S)-tyrosine | 61.7 ± 17.5 | 211 ± 65 | 46.6 |
| carbamate | | | |
| Galantamine phenylalanine | 54.0 ± 47.4 | 118 ± 48 | 26.0 |
| carbamate | | | |
| Galantamine phenylalanine | 20.4 ± 10.2 | 98.1 ± 47.5 | 21.7 |
| carbamate methyl ester | | | |
| Galantamine-(S)-tryptophan | 16.7 ± 2.5 | 66.8 ± 15.9 | 14.7 |
| carbamate | | | |
| Galantamine glutaryl (S) leucine | 16.4 ± 1.63 | 72 ± 5 | 10.0 |
| ester | | | |
| Galantamine valine carbamate | 13.8 ± 5.5 | 41.9 ± 8.0 | 9.2 |
| Galantamine para-amino methyl | 12.7 ± 4.3 | 46.7 ± 25 | 7.0 |
| benzoic acid carbamate | | | |
| Galantamine para-amino | 7.4 ± 1.0 | 24.2 ± 3 | 3.5 |
| nicotinic acid carbamate | | | |
| Galantamine valine carbamate | 3.25 ± 0.33 | 12.0 ± 4.1 | 2.6 |
| methyl ester | | | |
| Galantamine para amino benzoic | 4.6 ± 0.6 | 15 ± 1 | 2.0 |
| acid carbamate | | | |

PABA = para-amino benzoic acid

TABLE 7
Comparative pharmacokinetic parameters for galantamine in the monkey following
oral administration of 1 mg equivalent galantamine free base/kg of galantamine
itself or various amino acid prodrugs of galantamine

| | Cₘₐₓ | AUC | Relative oral |
|---|---|---|---|
| Compound Name | (mean ng/mL ± sd) | (mean ng · h/mL ± sd) | Bioavailability (%) |
| Galantamine HBr | 127 ± 6 | 258 ± 56 | — |
| Galantamine [glutaryl-PABA] | 18 ± 2 | 94 ± 20 | 39 |
| ester | | | |
| Galantamine-[succinyl-(S)- | 11.3 ± 6.9 | 50 ± 35 | 20 |
| valine] ester | | | |
| Galantamine glutaryl (S) leucine | 8.87 ± 2.3 | 36 ± 16 | 14.5 |
| ester | | | |
| Galantamine para-amino | 3.0 ± 1.34 | 5.5 ± 3.0 | 2.0 |
| nicotinic acid carbamate | | | |
| Galantamine para-amino methyl | 2.35 ± 0.25 | 5.4 ± 0.4 | 1.7 |
| benzoic acid carbamate | | | |
| Galantamine para amino benzoic | 1.61 ± 0.6 | 3 ± 1 | 1.5 |
| acid carbamate | | | |

PABA = para-amino benzoic acid

### Example 9

### Study on the Plasma Persistence of Galantamine from Galantamine Succinyl Valine Ester in the Dog and Monkey

A more detailed examination was conducted to study the sustainment or maintenance of blood levels of galantamine following administration of the galantamine succinyl valine ester prodrug as compared to the sustainment or maintenance of blood levels of galantamine when administered in the parent drug form in dogs and monkeys

Test substances (i.e., galantamine (parent drug) or galantamine succinyl valine ester (prodrug)) were administered by oral gavage to groups of five or six beagle dogs or cynomolgus monkeys. Blood samples were taken at various times after dosing and submitted to analysis for the parent drug using a validated LC-MS-MS assay.

Pharmacokinetic parameters derived from the plasma analytical data, including t½, AUC, absolute bioavailability, etc., were determined using the WinNonlin® data analysis program.

Results

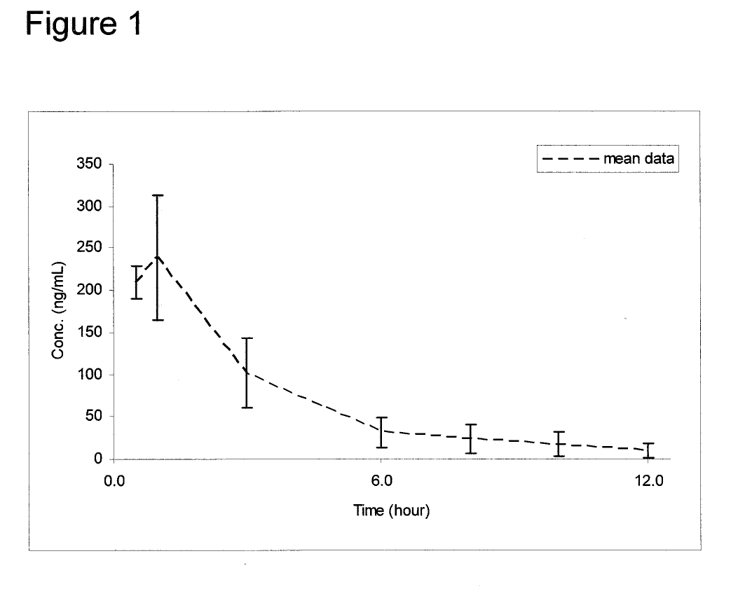

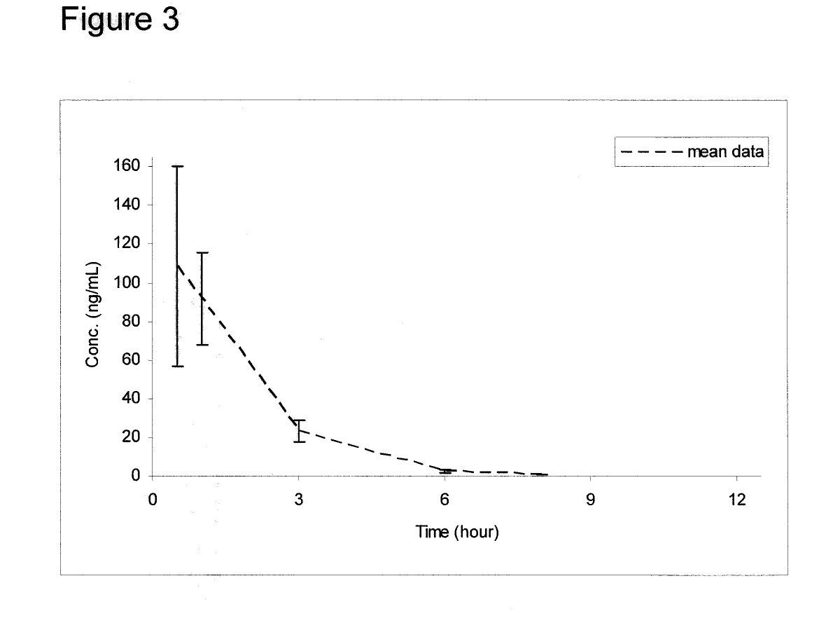

Results are shown in Tables 8, 9, 10 & 11 and FIGS. 1, 2, 3 & 4.

In dogs, the mean T_{>50% Cmax} value (the period for which plasma drug concentrations remained at or above 50% of their maximum values) for galantamine was 2.26±0.29 h after giving the drug itself. In contrast, the T_{>50% Cmax} value after giving the succinyl valine ester prodrug, was 6.28±0.98 h, almost three-fold longer.

In monkeys, the mean T_{>50% Cmax} value for galantamine was 1.5±0.39 h after administering the parent drug itself. In contrast, the T_{>50% Cmax} value, after giving the succinyl valine ester prodrug, was 4.85±0.98 h, over three-fold longer

These increased sustainments of plasma drug levels should enable less frequent drug administration further serving to minimize adverse GI events (vomiting and diarrhea) and unintentional drug loss, thus improving patient response and compliance.

TABLE 8
Pharmacokinetics of galantamine following oral administration of 1 mg/kg
galantamine HBr to the female beagle dog

| PK parameter | 100 | 102 | 104 | 110 | 112 | Mean | SD |
|---|---|---|---|---|---|---|---|
| T_{1/2} | 1.47 | 1.74 | 1.47 | 2.37 | 3.29 | 2.07 | ±0.77 |
| Tₘₐₓ | 0.5 | 1.0 | 0.5 | 0.5 | 1.0 | 0.70 | ±0.27 |
| Cₘₐₓ | 236 | 226 | 211 | 217 | 362 | 250 | ±63 |
| AUC | 740 | 763 | 512 | 688 | 1344 | 809 | ±315 |
| T_{>50%} _{Cmax} | 2.56 | 1.85 | 2.47 | 2.07 | 2.33 | 2.26 | ±0.29 |

TABLE 9
Pharmacokinetics of galantamine following oral administration of 1 mg/kg
galantamine succinyl-(S)-valine ester TFA (expressed as galantamine
free base equivalents) to the female beagle dog

| PK parameter | 100 | 102 | 104 | 110 | 112 | Mean | SD |
|---|---|---|---|---|---|---|---|
| T_{1/2} | 4.20 | 3.29 | 3.66 | 3.61 | 5.75 | 4.10 | ±0.98 |
| Tₘₐₓ | 3 | 3.0 | 3.0 | 3.0 | 3.0 | 3.00 | ±0 |
| Cₘₐₓ | 17 | 25 | 53 | 52 | 40 | 37 | ±16 |
| AUC | 132 | 178 | 391 | 365 | 400 | 293 | ±128 |
| T_{>50%} _{Cmax} | 5.16 | 6.22 | 6.77 | 5.62 | 7.65 | 6.28 | ±0.98 |
| Fᵣₑₗₐₜᵢᵥₑ | 18% | 23% | 76% | 53% | 30% | 40% | ±24 |
| C_{max relative} | 7.3% | 11% | 25% | 24% | 11% | 16% | ±8.3 |

TABLE 10
Pharmacokinetics of galantamine following oral administration of 1 mg/kg
galantamine HBr to the female cynomolgus monkey

| PK parameter | 964 | 966 | 968 | 970 | 972 | Mean | SD |
|---|---|---|---|---|---|---|---|
| T_{1/2} | 0.86 | 0.96 | 1.03 | 0.98 | 0.96 | 0.96 | ±0.06 |
| Tₘₐₓ | 0.5 | 0.5 | 0.5 | 0.5 | 1.0 | 0.60 | ±0.22 |
| Cₘₐₓ | 129 | 132 | 121 | 144 | 69 | 119 | ±29 |
| AUC | 221 | 322 | 232 | 241 | 154 | 234 | ±60 |
| T_{>50%} _{Cmax} | 2.08 | 1.60 | 1.30 | 1.01 | 1.50 | 1.50 | ±0.39 |

TABLE 11
Pharmacokinetics of galantamine following oral administration of 1 mg/kg
galantamine succinyl-(S)-valine ester TFA (expressed as galantamine free
base equivalents) to the female cynomolgus monkey

| PK parameter | 964 | 966 | 968 | 970 | 972 | Mean | SD |
|---|---|---|---|---|---|---|---|
| T_{1/2} | 13.70 | 2.48 | 3.02 | 3.00 | 37.07 | 11.86 | 14.86 |
| Tₘₐₓ | 3 | 3.0 | 3.0 | 3.0 | 3.0 | 3.00 | 0 |
| Cₘₐₓ | 5 | 22 | 8 | 9 | 6 | 10 | 7 |
| AUC | 54 | 120 | 48 | 38 | 22 | 57 | 37 |
| T_{>50%} _{Cmax} | 5.56 | 5.03 | 4.47 | >5.1 | >4.1 | 4.85 | 0.57 |
| Fᵣₑₗₐₜᵢᵥₑ | 25% | 37% | 21% | 16% | 15% | 23% | 9% |
| C_{max relative} | 4% | 17% | 7% | 6% | 8% | 8% | 5% |

### Example 10

### Comparative In Vitro Assessment of Human Acetylcholine Esterase Inhibition by Galantamine and Various Amino Acid Prodrugs

Methodology

TABLE 12
Assay used to measure human acetylcholine esterase inhibition

| | | Reference | |
|---|---|---|---|
| Assay | Origin | Compound | Bibliography |
| Acetylcholin- | human recombinant | neostigmine | Ellman |
| esterase (h) | (HEK-293 cells) | | et al. (1961) |

Experimental Conditions

TABLE 13
Experimental Conditions used for assay

| | Substrate/ | | Reaction | Method of |
|---|---|---|---|---|
| Assay | Tracer | Incubation | Product | Detection |
| Acetylcholin- | AMTCh | 30 min./ | thio- | Photometry |
| esterase (h) | (50 μM) | 37° C. | conjugate | |

Analysis and Expression of Results

The results are expressed as a percent of control specific activity ((measured specific activity/control specific activity)×100) obtained in the presence of the test compounds.

The IC₅₀ values (concentration causing a half-maximal inhibition of control specific activity), and Hill coefficients (nH) were determined by non-linear regression analysis of the inhibition curves generated with mean replicate values using Hill equation curve fitting (Y=D+[(A−D)/(1+(C/C₅₀)^{nH})], where Y=specific activity, D=minimum specific activity, A=maximum specific activity, C=compound concentration, C₅₀=IC₅₀, and nH=slope factor).

This analysis was performed using software developed at Cerep (Hill software) and validated by comparison with data generated by the commercially available software SigmaPlot® 4.0 software.

Results

TABLE 14
Effects of various galantamine prodrugs
on human acetyl choline esterase

| | IC₅₀ for inhibition of human | |
|---|---|---|
| Galantamine prodrug | acetyl choline esterase | |
| Galantamine | 1.8 | μM |
| Galantamine phenylalanine | No inhibition observed | |
| carbamate | | |
| Galantamine succinyl valine ester | >100 | μM |
| Galantamine glutaryl PABA ester | >100 | μM |
| Galantamine glutaryl (S) leucine ester | >100 | μM |
| Galantamine para amino benzoic acid | 32 | μM |
| carbamate | | |
| Galantamine meta amino benzoic acid | 26 | μM |
| carbamate | | |
| Galantamine para-amino methyl | 46 | μM |
| benzoic acid carbamate | | |
| Galantamine para-amino nicotinic acid | 55 | μM |
| carbamate | | |

The results presented in Table 14 show the apparent IC₅₀ value for galantamine of 1.8 μM from this study to be somewhat less than previously reported for human erythrocytes (0.35 μM), but was nevertheless within the expected ± of 0.5 log units for such estimations.

In contrast to galantamine, the phenylalanine carbamate prodrug was apparently without activity while both the succinyl valine ester and the glutaryl PABA ester conjugates of galantamine, demonstrated significantly less inhibitory actions toward human acetylcholine esterase. This implies that when in contact with the gut wall they may be less likely to directly elicit a cholinergic response Later studies presented as examples 11, 12, and 13 will show the significance of this in relation to the emetic effects of galantamine.

### Example 11

### Ex Vivo Assessment of the Effects of Galantamine and its Conjugates Galantamine Succinyl Valine Ester on Rabbit and Human Stomach Circular Smooth Muscle Preparations

In order to determine whether galantamine may have a direct effect on gastric smooth muscle and potentially thereby elicit emesis by this mechanism, an investigation of the effects of the drug and its succinyl valine ester prodrug initially using rabbit and later human stomach tissue was undertaken.

Methodology

Strips of rabbit or human stomach smooth muscle (mucosa intact) cut from antral region and mounted between platinum ring electrodes.

The tissue was stretched to steady tension of ˜1 g and changes in force production were recorded using sensitive transducers.

The optimal voltage for stimulation was determined while the tissue was paced with electrical field stimulation (EFS) at 14 Hz, with a pulse width of 0.5 msec. Trains of pulses occurred for 20 seconds, every 50 seconds.

EFS at optimal voltage was continued throughout the protocol (stable responses=“baseline measurement of EFS”).

3 test conditions:

(1) vehicle (deionized water, added at equivalent volume additions to test articles)

(2) Galantamine at 6 concentrations (100 nM, 1 μM, 3 μM, 10 μM, 30 μM, 100 μM)

(3) Galantamine succinyl valine ester at 6 concentrations (100 nM, 1 μM, 3 μM, 10 μM, 30 μM, 100 μM)

Following 10 minutes of baseline EFS, the first addition of test article or vehicle (deionized water) was performed. Test concentrations were added in a non-cumulative manner with PSS washes between each addition.

Addition of TTX (Na⁺ channel blocker) was then carried out to confirm EFS responses elicited via nerve stimulation. EFS was then stopped and then acetylcholine (1 μM) was added to confirm heck tissue viability at end of study. The response of the muscle preparations (change in force production) was measured for each test compound and concentration.

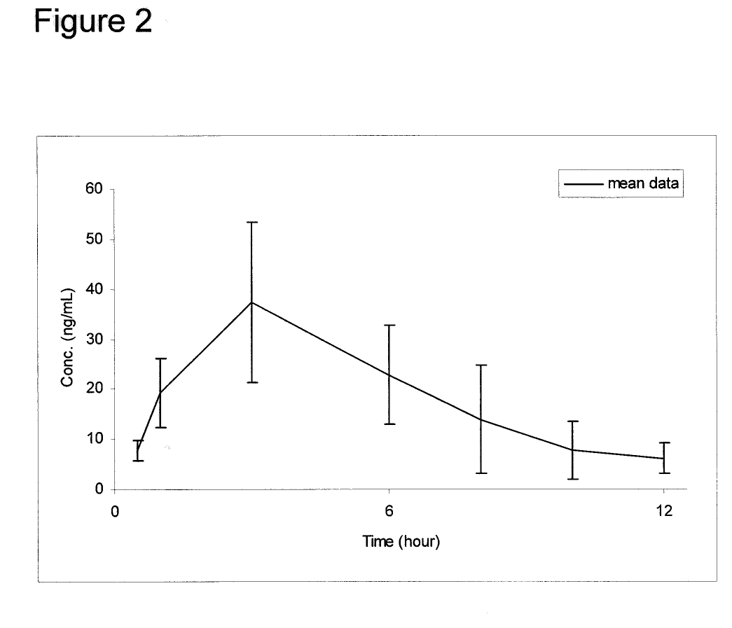

The results of this experiment, while showing evidence of a dose response for galantamine itself stimulating smooth muscle contractions, also indicate a complete absence of any such effect with the succinyl valine ester prodrug (FIGS. 1 and 2). This suggests the prodrug may have little action in vivo in stimulating contractions of stomach smooth muscle and thereby emesis.

### Example 12

### Investigation of the Direct Intragastric Emetic Actions of Galantamine in the Sprague Dawley Rat and Avoidance by Use of a Selected Prodrug, Galantamine Succinyl Valine Ester

In order to confirm that direct intragastric cholinergic effects of galantamine were responsible for the emetic actions of the drug, a comparison was made of the effects of drug after either parenteral (subcutaneous) or oral dosing. Studies were subsequently carried out to investigate the effects of a candidate prodrug galantamine succinyl valine ester. Because rats do not possess a vomiting reflex, measurement of the so-called PICA behavior (i.e., consumption of non nutritive material (e.g., kaolin)) was used as a surrogate for emesis. This is a well established model for this purpose in the rat (Takeda N et al (1993) 45 817-21).

Methodology

Initially the maximum tolerated oral and subcutaneous dose levels of galantamine were established in the rat. Once determined, a comparison was made of the effects of these doses on kaolin consumption over a 0-96 hour period in 24 h increments.

Subsequently a comparison was made of kaolin consumption after administration of a single oral dose of either galantamine (40 mg/kg) or various doses of galantamine succinyl valine ester, (GSVE) up to 47 mg galantamine free base content/kg, to rats.

In detail groups of 10 male Sprague Dawley rats were habituated to kaolin for 3 days, then singly housed in grid-bottomed cages and habituated for a further 2 days prior to dosing. On the day of drug administration, the animals were food-deprived for 1 h prior to dosing. At t=0, rats were orally dosed with 1% methylcellulose vehicle or LiCL 130 mg base/kg (positive control) or galantamine 40 mg free base/kg or galantamine succinyl valine ester 11.75 23.5 and 47 mg galantamine base content/kg po. Access to a weighed quantity of food and kaolin was then restored. Food and kaolin was weighed 24, 48, 72 and 96 h after dosing.

Results

As shown in Table 15, the initial assessment of the comparative acute toxicity of orally (po) and subcutaneously (sc) administered doses of galantamine showed that 3.5 mg/kg sc elicited much the same overt clinical signs as did 40 mg/kg po. These doses were therefore selected as the doses to be used for the comparative assessment of effects on PICA behavior.

As seen in Table 16, kaolin consumption over the 96 h post drug administration was found to be significantly higher in the animals orally dosed with the drug at 40 mg/kg being strongly indicative of emetic-like activity. By contrast the subcutaneously dosed rats showed no increase at all in kaolin consumption compared with controls over the whole 96 h period suggesting that when the drug is given by this route it is not emetic. Importantly the lack of kaolin consumption after the sc dose was not simply a reflection of drug induced inappetence since food consumption was indistinguishable between the oral and sc groups

A subsequent study comparing the effects of orally administered galantamine itself at 40 mg/kg or the succinyl valine ester prodrug up to 47 mg (galantamine free base content)/kg showed the former, once again, to induce marked PICA behavior in the rat. The results presented in Table 17 show little evidence for any increase in kaolin consumption when the prodrug was administered (compared with that seen after the vehicle alone). The comparative consumption of kaolin over 96 h was 4.69±2.43, 0.91±0.45 & 0.75±0.27 for galantamine (40 mg/kg), galantamine succinyl valine ester (47 mg/kg) and vehicle respectively This suggests that the emetic properties of galantamine may have been much reduced following administration of this prodrug.

TABLE 15
Effect of galantamine 3.5 mg/kg sc and 40 mg/kg po on rat behavior

| Time after | | | |
|---|---|---|---|
| dosing | | Galantamine 3.5 mg/kg sc | Galantamine 40 mg/kg po |
| 2 | min post dose | Rat 1: purposeless chewing, mouth | |
| | | movements, flattened rostral body posture | |
| | | Rat 3: purposeless chewing, mouth | |
| | | movements, flattened rostral body posture | |
| 5 | min post-dose | Rat 1: purposeless chewing, mouth movements, | |
| | | flattened rostral body posture + yawning | |
| | | Rat 3: As per 2 min | |
| 10 | min post-dose | Rat 1: Tremors, flattened rostral body | Rat 6: Quiescent, purposeless |
| | | posture, body twitches | chewing, flattened body posture |
| | | Rat 3: purposeless chewing, mouth | Rat 7: Quiescent, purposeless chewing, |
| | | movements, flattened rostral body | flattened body posture, grooming |
| | | posture, shaking, muscle twitching | |
| 20 | min post-dose | Rat 1: Quiescent, flattened body posture, | Rate 6 as per 10 min + arching |
| | | purposeless chewing, grooming - | |
| | | symptoms less intense | |
| | | Rat 3: Some ambulation restored symptoms | Rat 7: grooming + purposeless |
| | | less intense, purposeless chewing | chewing + flattened body posture, |
| | | | yawning and tremor |
| 30 | min post-dose | Rat 1: Quiescent, flattened body posture, | Rat 6: quiescent, purposeless chewing, |
| | | purposeless chewing, grooming - | yawning, lacrymation |
| | | symptoms less intense | |
| | | Rat 3: Some ambulation restored | Rat 7: |
| | | symptoms less intense, purposeless | |
| | | chewing, yawning | |
| 45 | min pos-dose | Rat 1: food consumption and more | Rat 6: flattened body posture, chewing, |
| | | normal appearance | tremor |
| | | Rat 3: | Rat 7: grooming, sawdust consumption |
| 1 | h | Rat 1: Normal | Rat 6: flattened body posture, tremor |
| | | | ceased, some ambulation, yawning, |
| | | | purposeless chewing |
| | | Rat 3: Normal | Rat 7: Grooming, sawdust |
| | | | consumption, purposeless chewing |
| 1.5 | h | | Rat 6: Flattened posture but |
| | | | ambulation |
| | | | Rat 7: quiet, falttened posture |
| 2 | h | | Rat 6: much improved |
| | | | Rat 7: much improved |
| 3 | h | | Rat 6: normal |
| | | | Rat 7: normal |

TABLE 16
Effect of a single oral dose of galantamine 40 mg base/kg
or a single subcutaneous dose of 3.5 mg base/kg on
rat kaolin consumption (g) over 24 h post-dose

| Rat | | Galantamine 3.5 | Galantamine 40 |
|---|---|---|---|
| number | Vehicle | mg/kg sc | mg/kg po |
| 1 | 0.08 | 0.05 | 3.11 |
| 2 | 0.03 | 0.00 | 1.03 |
| 3 | 0.10 | 0.67 | 1.06 |
| 4 | 0.05 | 0.36 | 0.35 |
| 5 | 0.04 | 0.88 | 0.02 |
| 6 | 0.00 | 0.08 | 3.14 |
| 7 | 0.84 | *1.57 | 4.47 |
| 8 | 0.74 | 0.00 | 1.62 |
| 9 | 0.07 | 0.21 | 0.16 |
| 10 | 0.43 | 0.00 | 2.93 |
| Mean | 0.24 | 0.25 | 1.79 |
| SD | 0.32 | 0.32 | 1.53 |

*Omitted from analysis as a statistical outlier

TABLE 17
Cumulative mean ± SD kaolin consumption (g) at different times
time after dosing galantamine or galantamine succinyl valine ester to rats

| | Collection period | | | |
|---|---|---|---|---|
| Treatment | 24 h | 48 h | 72 h | 96 h |
| Vehicle | 0.20 ± 0.27 | 0.51 ± 0.40 | 0.61 ± 0.31 | 0.75 ± 0.27 |
| GSVE * 11.75 mgbase **/kg | 0.14 ± 0.20 | 0.29 ± 0.24 | 0.48 ± 0.27 | 0.57 ± 0.40 |
| GSVE * 23.5 mg base **/kg | 0.31 ± 0.53 | 0.61 ± 0.69 | 0.95 ± 0.96 | 1.25 ± 1.31 |
| GSVE * 47 mg base **/kg | 0.15 ± 0.18 | 0.40 ± 0.18 | 0.63 ± 0.28 | 0.91 ± 0.45 |
| Galantamine 40 mg base/kg | 2.73 ± 1.4 *** | 3.36 ± 1.68 *** | 4.07 ± 1.88 *** | 4.69 ± 2.43 *** |
| Positive control - LiCl 130 | 3.33 ± 1.98 *** | 3.71 ± 2.22 *** | 4.42 ± 2.82 *** | 4.82 ± 3.11 *** |
| mg base/kg | | | | |

* GSVE = galantamine succinyl valine ester
** Dose of prodrug refers to galantamine free base content/kg
*** P < 0.001 Dunnett's test difference from vehicle dosed animals

### Example 13

### In Vivo Assessment of the Effects of Galantamine and its Conjugates on Emetic Activity in the Ferret

The classic model for preclinical assessment of emetic activity employs the ferret and involves assessing the number and time of onset of retches and vomits over a 2 h period following administration of the drug or vehicle. A comparison was made of the effects of either galantamine itself or galantamine succinyl valine ester in this model.

Methodology

Male ferrets were fasted overnight and up to the end of the 2 hr. observation period post dosing. The test compound was administered p.o. prior to observation at a dose expressed in mg/kg with respect to weight of galantamine free base content using an aqueous vehicle volume of 5 mL/kg. Animals responding to the emetic effects of galantamine were then used in assessment of the effects of the prodrugs. The administered dose of prodrug was based on the bioavailability of galantamine from these compounds, in the dog, relative to that of the drug itself. For example, galantamine phenylalanine carbamate ester was given at 4× the galantamine dose based on a bioavailability in the dog of 25%. Similarly galantamine succinyl valine ester was given at 2× the galantamine dose based on this prodrug having only half the bioavailability of the drug itself. Galantamine succinyl valine ester was given at 1× since it showed comparable bioavailability with galantamine. The frequency and timing of retching and vomiting was recorded over a period of 2 hr. post dosing

Results

The results presented in Tables 18 & 19, show that after galantamine treatment at 20 mg (free base)/kg not all the animals retched or vomited, but 55% & 40%, respectively, of those dosed did so. Oral administration of the valine ester (valgalantamine) at a similar molar dose showed a somewhat lesser effect (45% and 18% respectively). However, no retching or vomiting at all was observed in any of the animals dosed orally with the succinyl valine ester at 40 mg/galantamine (free base equivs)/kg. This is consistent with the previous work, firstly showing much reduced acetyl choline esterase activity and subsequently the lack of effect in the isolated organ bath work using rabbit or human stomach smooth muscle and finally in the rat PICA model were no effects were seen.

TABLE 18
Effects of galantamine on retching and vomiting in the ferret

| | Ani- | Total number of | | Time (min) to | |
|---|---|---|---|---|---|
| | mal | individual incidences of: | | onset of: | |
| Treatment | No. | Retching | Vomiting | Retching | Vomiting |
| Galantamine | 1 | 3 | 2 | 6 | 6 |
| HBr | 2 | 0 | 0 | >120 | >120 |
| 20 mg/kg | 3 | 22 | 1 | 6 | 41 |
| | 4 | 21 | 4 | 33 | 33 |
| | 5 | 29 | 1 | 3 | 75 |
| | 6 | 18 | 1 | 5 | 5 |
| | 7 | 0 | 0 | >120 | >120 |
| | 8 | 31 | 4 | 41 | 37 |
| | 9 | 3 | 0 | 5 | >120 |
| | 10 | 0 | 0 | >120 | >120 |
| | 11 | 0 | 0 | >120 | >120 |
| | 12 | 6 | 0 | 11 | >120 |
| | 13 | 0 | 1 | 10 | 10 |
| | 14 | 0 | 0 | >120 | >120 |
| | 15 | 0 | 0 | >120 | >120 |
| | 16 | 2 | 0 | 7 | >120 |
| | 17 | 0 | 0 | >120 | >120 |
| | 18 | 0 | 0 | >120 | >120 |
| | 19 | 5 | 1 | 5 | 5 |
| | 20 | 13 | 1 | 7 | 13 |

TABLE 19
Effects galantamine valine ester (valgalantamine) and galantamine
succinyl valine ester on retching and vomiting in the ferret

| | Ani- | Total number of | | Time (min) to | |
|---|---|---|---|---|---|
| | mal | individual incidences of: | | onset of: | |
| Treatment | No. | Retching | Vomiting | Retching | Vomiting |
| Galantamine | 1 | 2 | 0 | 6 | >120 |
| valine ester | 3 | 0 | 0 | >120 | >120 |
| tartrate (val- | 4 | 22 | 1 | 35 | 44 |
| galantamine) | 5 | 0 | 0 | >120 | >120 |
| 20 mg/kg | 6 | 2 | 0 | 106 | >120 |
| | 8 | 0 | 0 | >120 | >120 |
| | 9 | 0 | 0 | >120 | >120 |
| | 12 | 3 | 0 | 11 | >120 |
| | 16 | 14 | 3 | 9 | 9 |
| | 19 | 0 | 0 | >120 | >120 |
| | 20 | 0 | 0 | >120 | >120 |
| Mean | | 3.9 | | | |
| Galantamine | 1 | 0 | 0 | >120 | >120 |
| succinyl | 3 | 0 | 0 | >120 | >120 |
| valine ester | | | | | |
| 40 mg/kg | 4 | 0 | 0 | >120 | >120 |
| as base | 5 | 0 | 0 | >120 | >120 |
| | 8 | 0 | 0 | >120 | >120 |
| | 20 | 0 | 0 | >120 | >120 |
| Mean | | 0 | 0 | >120 | >120 |

Patents, patent applications, publications, product descriptions, and protocols which are cited throughout this application are incorporated herein by reference in their entireties. The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A compound of Formula 1: 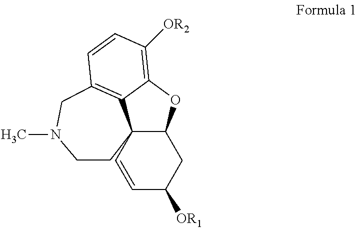
or a pharmaceutically acceptable salt thereof, wherein
R₁ is H, 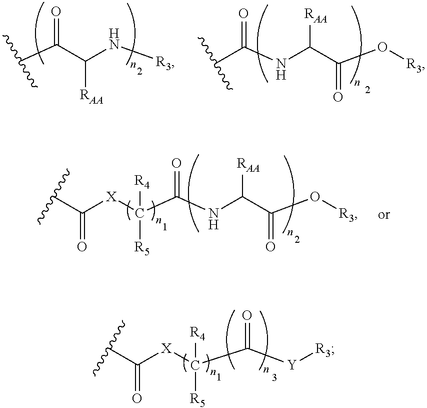
R₂ is H, CH₃, 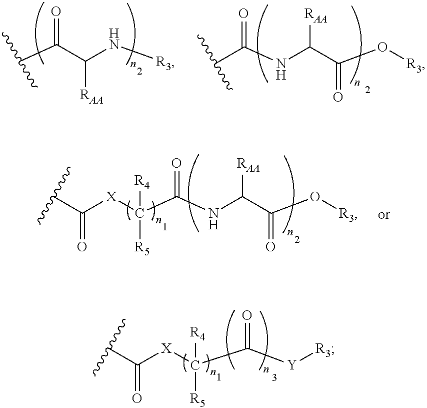
each occurrence of R₃ is independently hydrogen, a substituted alkyl group or an unsubstituted alkyl group;
each occurrence of R₄, R₅, R₆, and R₇ is independently selected from hydrogen, 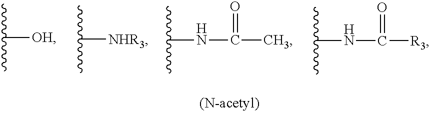
a substituted alkyl group, or an unsubstituted alkyl group;
each occurrence of n₁ is independently 0, 1, 2, 3, 4, 5, 6, 7, 8, 9, 10, 11, 12, 13, 14, 15, or 16;
each occurrence of n₂ is independently 1, 2, 3, 4, 5, 6, 7, 8, or 9;
each occurrence of n₃ is independently 0 or 1;
each occurrence of n₄ is independently 0, 1, 2, 3, 4, 5, 6, 7, 8, 9, 10, 11, 12, 13, 14, 15, or 16;
each occurrence of n₅ is independently 0 or 1;
each occurrence of R_{AA} is independently a proteinogenic or non-proteinogenic amino acid side chain;
each occurrence of X is independently (—NH—), (—O—), or absent;
each occurrence of X′ is independently (—NH—) (—O—) or absent;
each occurrence of Y is independently 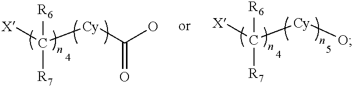
and
each occurrence of Cy is independently a 5- or 6-membered cycloalkyl, 5- or 6-membered heterocycle, 5- or 6-membered aryl, or 5- or 6-membered heteroaryl, wherein Cy optionally has fused thereto a second ring which is a 5- or 6-membered heterocycle, 5- or 6-membered cycloalkyl 5- or 6-membered aryl or a 5- or 6-membered heteroaryl ring.

2. The compound of claim 1, wherein R₁ is

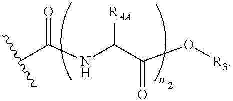

3. The compound of claim 1, wherein R₂ is

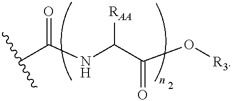

4. The compound of claim 2 or 3, wherein each occurrence of R_{AA} is independently the amino acid side chain of valine, phenylalanine, tryptophan, or tyrosine.

5. The compound of claim 4, wherein R₂ is CH₃; n₂ is 1; and each occurrence of R₃ is independently H or an alkyl group.

6. The compound of claim 1, wherein R₁ is

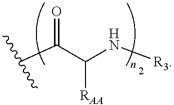

7. The compound of claim 1, wherein R₂ is

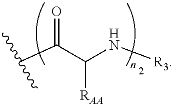

8. The compound of claim 6 or 7, wherein each occurrence of R_{AA} is independently the amino acid side chain of valine, phenylalanine, tryptophan, or tyrosine.

9. The compound of claim 6 or 7, wherein R₃ is H and R_{AA} is the amino side chain of valine.

10. The compound of claim 9, wherein R₂ is CH₃; n₂ is 1; and R₃ is H.

11. The compound of claim 1, wherein R₁ is

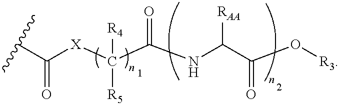

12. The compound of claim 1, wherein R₂ is

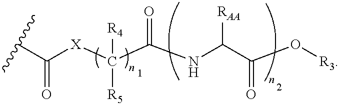

13. The compound of claim 11 or 12, wherein X is absent.

14. The compound of claim 13, wherein each occurrence of R_{AA} is independently the amino acid side chain of valine or leucine.

15. The compound of claim 14, wherein each occurrence of R₃, R₄, and R₅ is H; n₁ is 2 or 3; and n₂ is 1.

16. The compound of claim 15, wherein n₁ is 2 and R_{AA} is the amino side chain of valine.

17. The compound of claim 1, wherein R₁ is

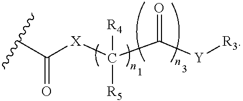

18. The compound of claim 1, wherein R₂ is

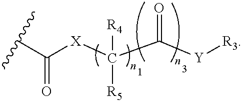

19. The compound of claims 17 and 18, wherein Y is

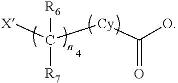

20. The compound of claim 19, wherein n₁ is 3; n₃ is 1; n₄ is 0; X is absent;
each occurrence of X′ is independently O or NH; each occurrence of R₃ is independently H or an alkyl group; R₄ and R₅ are H, and each occurrence of Cy is independently an aryl.

21. The compound of claim 20, wherein R₃ is H and X′ is O.

22. The compound of claim 19, wherein n₁ and n₃ are 0; n₄ is 1; X′ is NH; R₃, R₆, and R₇ are H; and Cy is aryl.

23. The compound of claim 19, wherein n₁, n₃, and n₄ are 0; X′ is absent; R₃ is H; and Cy is aryl.

24. The compound of claim 19, wherein n₁, n₃, and n₄ are 0; X′ is NH; R₃ is H; and Cy is aryl.

25. The compound of claim 19, wherein n₁, n₃, and n₄ are 0; X′ is NH; R₃ is H; and Cy is a heteroaryl.

26. The compound of claim 1, wherein the formula is Formula 1a: 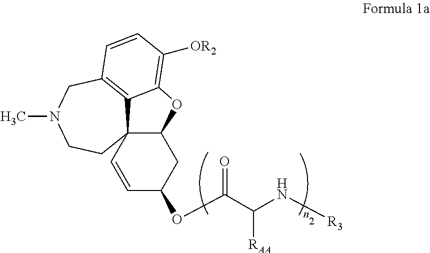
or a pharmaceutically acceptable salt thereof, wherein
R₂ is H, CH₃, 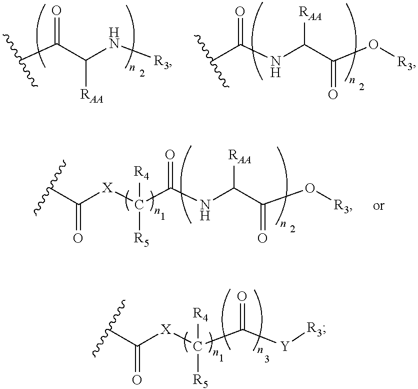
each occurrence of R₃ is independently hydrogen, a substituted alkyl group or an unsubstituted alkyl group;
each occurrence of R₄, R₅, R₆, and R₇ is independently selected from hydrogen, 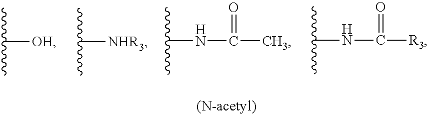
a substituted alkyl group, or an unsubstituted alkyl group;
each occurrence of n₁ is independently 0, 1, 2, 3, 4, 5, 6, 7, 8, 9, 10, 11, 12, 13, 14, 15, or 16;
each occurrence of n₂ is independently 0, 1, 2, 3, 4, 5, 6, 7, 8, or 9;
each occurrence of n₃ is independently 0 or 1;
each occurrence of n₄ is independently 0, 1, 2, 3, 4, 5, 6, 7, 8, 9, 10, 11, 12, 13, 14, 15, or 16;
each occurrence of n₅ is independently 0 or 1;
each occurrence of R_{AA} is independently a proteinogenic or non-proteinogenic amino acid side chain;
each occurrence of X is independently (—NH—), (—O—), or absent;
each occurrence of X′ is independently (—NH—), (—O—), or absent;
each occurrence of Y independently is 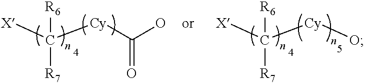
and
each occurrence of Cy is independently a 5- or 6-membered cycloalkyl, 5- or 6-membered heterocycle, 5- or 6-membered aryl, or 5- or 6-membered heteroaryl, wherein Cy optionally has fused thereto a second ring which is a 5- or 6-membered heterocycle, 5- or 6-membered cycloalkyl 5- or 6-membered aryl or a 5- or 6-membered heteroaryl ring.

27. The compound of claim 1, wherein the formula is Formula 1b: 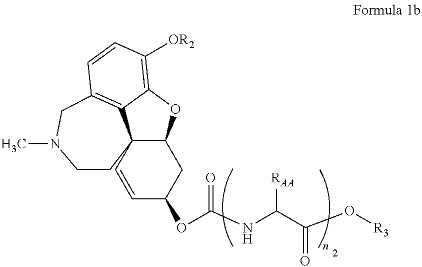
or a pharmaceutically acceptable salt thereof, wherein
R₂ is H, CH₃, 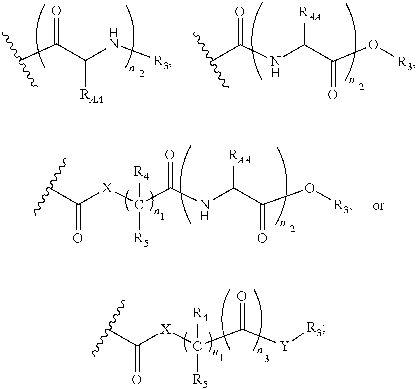
each occurrence of R₃ is independently hydrogen, a substituted alkyl group or an unsubstituted alkyl group;
each occurrence of R₄, R₅, R₆, and R₇ is independently selected from hydrogen, 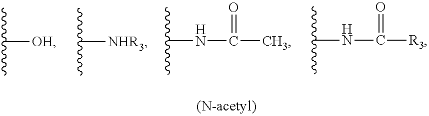
a substituted alkyl group, or an unsubstituted alkyl group;
each occurrence of n₁ is independently 0, 1, 2, 3, 4, 5, 6, 7, 8, 9, 10, 11, 12, 13, 14, 15, or 16;
each occurrence of n₂ is independently 0, 1, 2, 3, 4, 5, 6, 7, 8, or 9;
each occurrence of n₃ is independently 0 or 1;
each occurrence of n₄ is independently 0, 1, 2, 3, 4, 5, 6, 7, 8, 9, 10, 11, 12, 13, 14, 15, or 16;
each occurrence of n₅ is independently 0 or 1;
each occurrence of R_{AA} is independently a proteinogenic or non-proteinogenic amino acid side chain;
each occurrence of X is independently (—NH—), (—O—), or absent;
each occurrence of X′ is independently (—NH—), (—O—), or absent;
each occurrence of Y is independently 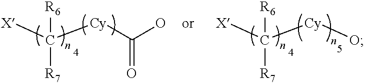
and
each occurrence of Cy is independently a 5- or 6-membered cycloalkyl, 5- or 6-membered heterocycle, 5- or 6-membered aryl, or 5- or 6-membered heteroaryl, wherein Cy optionally has fused thereto a second ring which is a 5- or 6-membered heterocycle, 5- or 6-membered cycloalkyl 5- or 6-membered aryl or a 5- or 6-membered heteroaryl ring.

28. The compound of claim 1, wherein the formula is Formula 1c: 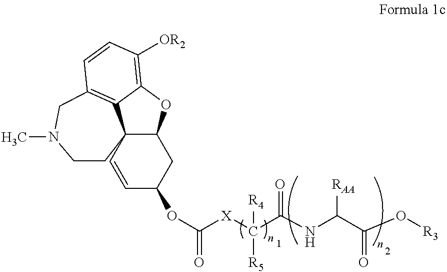
or a pharmaceutically acceptable salt thereof, wherein
R₂ is H, CH₃, 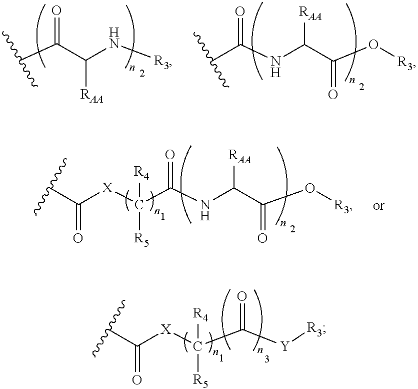
each occurrence of R₃ is independently hydrogen, a substituted alkyl group or an unsubstituted alkyl group;
Each occurrence of R₄, R₅, R₆, and R₇ is independently selected from hydrogen, 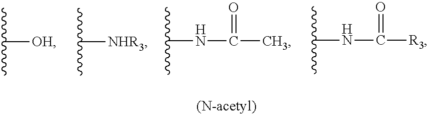
a substituted alkyl group, or an unsubstituted alkyl group;
each occurrence of n₁ is independently 0, 1, 2, 3, 4, 5, 6, 7, 8, 9, 10, 11, 12, 13, 14, 15, or 16;
each occurrence of n₂ is independently 0, 1, 2, 3, 4, 5, 6, 7, 8, or 9;
each occurrence of n₃ is independently 0 or 1;
each occurrence of n₄ is independently 0, 1, 2, 3, 4, 5, 6, 7, 8, 9, 10, 11, 12, 13, 14, 15, or 16;
each occurrence of n₅ is independently 0 or 1;
each occurrence of R_{AA} is independently a proteinogenic or non-proteinogenic amino acid side chain;
each occurrence of X is independently (—NH—), (—O—), or absent;
each occurrence of X′ is independently (—NH—), (—O—), or absent;
each occurrence of Y is independently 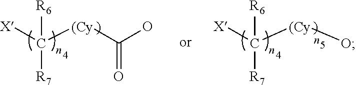
and
each occurrence of Cy is independently a 5- or 6-membered cycloalkyl, 5- or 6-membered heterocycle, 5- or 6-membered aryl, or 5- or 6-membered heteroaryl, wherein Cy optionally has fused thereto a second ring which is a 5- or 6-membered heterocycle, 5- or 6-membered cycloalkyl 5- or 6-membered aryl or a 5- or 6-membered heteroaryl ring.

29. The compound of claim 1, wherein the formula is Formula 1d: 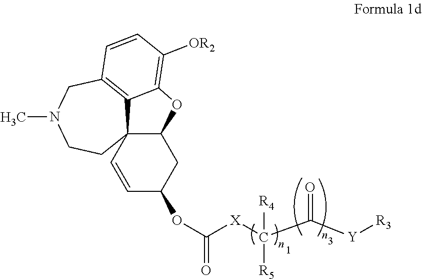
or a pharmaceutically acceptable salt thereof, wherein
R₂ is H, CH₃, 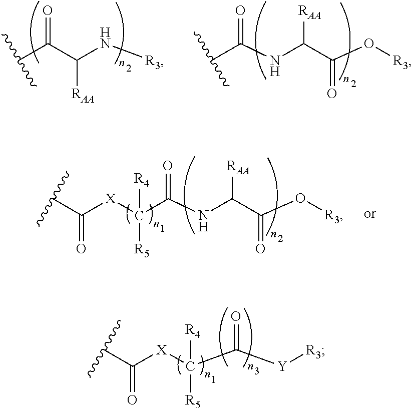
each occurrence of R₃ is independently hydrogen, a substituted alkyl group or an unsubstituted alkyl group;
each occurrence of R₄, R₅, R₆, and R₇ is independently selected from hydrogen, 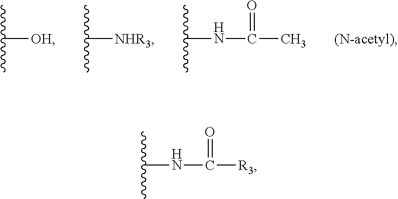
a substituted alkyl group, or an unsubstituted alkyl group;
each occurrence of n₁ is independently 0, 1, 2, 3, 4, 5, 6, 7, 8, 9, 10, 11, 12, 13, 14, 15, or 16;
each occurrence of n₂ is independently 0, 1, 2, 3, 4, 5, 6, 7, 8, or 9;
each occurrence of n₃ is independently 0 or 1;
each occurrence of n₄ is independently 0, 1, 2, 3, 4, 5, 6, 7, 8, 9, 10, 11, 12, 13, 14, 15, or 16;
each occurrence of n₅ is independently 0 or 1;
each occurrence of R_{AA} is independently a proteinogenic or non-proteinogenic amino acid side chain;
each occurrence of X is independently (—NH—), (—O—), or absent;
each occurrence of X′ is independently (—NH—), (—O—), or absent;
each occurrence of Y is 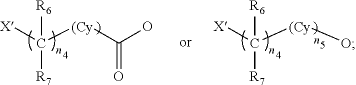
and
each occurrence of Cy is independently a 5- or 6-membered cycloalkyl, 5- or 6-membered heterocycle, 5- or 6-membered aryl, or 5- or 6-membered heteroaryl, wherein Cy optionally has fused thereto a second ring which is a 5- or 6-membered heterocycle, 5- or 6-membered cycloalkyl 5- or 6-membered aryl or a 5- or 6-membered heteroaryl ring.

30. The compound of claim 1, wherein the formula is Formula 1e: 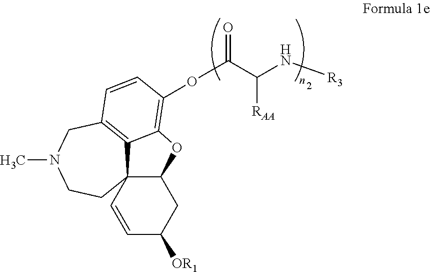
or a pharmaceutically acceptable salt thereof, wherein
R₁ is H, 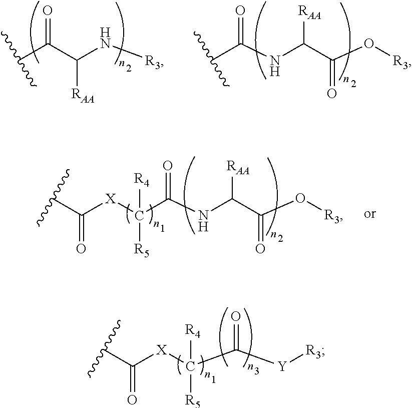
each occurrence of R₃ is independently hydrogen, a substituted alkyl group or an unsubstituted alkyl group;
each occurrence of R₄, R₅, R₆, and R₇ is independently selected from hydrogen, 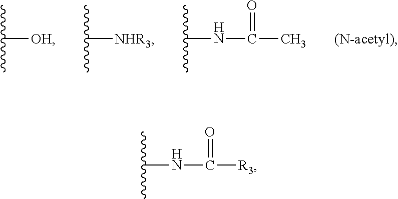
a substituted alkyl group, or an unsubstituted alkyl group;
each occurrence of n₁ is independently 0, 1, 2, 3, 4, 5, 6, 7, 8, 9, 10, 11, 12, 13, 14, 15, or 16;
each occurrence of n₂ is independently 1, 2, 3, 4, 5, 6, 7, 8, or 9;
each occurrence of n₃ is independently 0 or 1;
each occurrence of n₄ is independently 0, 1, 2, 3, 4, 5, 6, 7, 8, 9, 10, 11, 12, 13, 14, 15, or 16;
each occurrence of n₅ is independently 0 or 1;
each occurrence of R_{AA} is independently a proteinogenic or non-proteinogenic amino acid side chain;
each occurrence of X is independently (—NH—), (—O—), or absent;
each occurrence of X′ is independently (—NH—), (—O—), or absent;
each occurrence of Y is independently 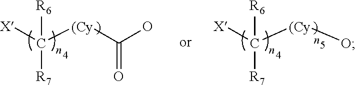
and
each occurrence of Cy is independently a 5- or 6-membered cycloalkyl, 5- or 6-membered heterocycle, 5- or 6-membered aryl, or 5- or 6-membered heteroaryl, wherein Cy optionally has fused thereto a second ring which is a 5- or 6-membered heterocycle, 5- or 6-membered cycloalkyl 5- or 6-membered aryl or a 5- or 6-membered heteroaryl ring.

31. The compound of claim 1, wherein the formula is Formula 1f 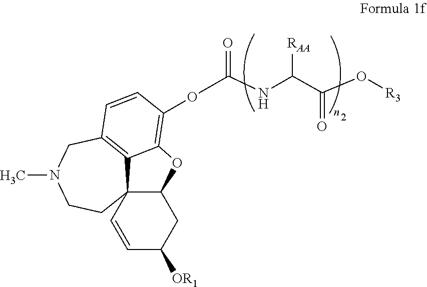
or a pharmaceutically acceptable salt thereof, wherein
R₁ is H, 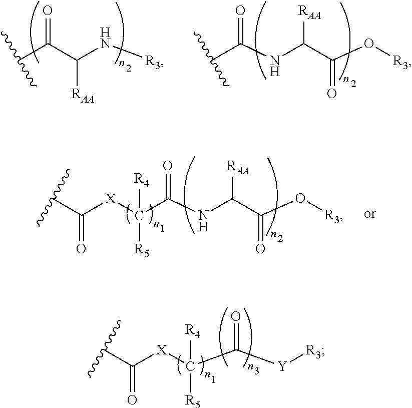
each occurrence of R₃ is independently hydrogen, a substituted alkyl group or an unsubstituted alkyl group;
each occurrence of R₄, R₅, R₆, and R₇ is independently selected from hydrogen, 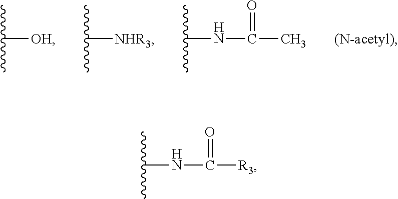
a substituted alkyl group, or an unsubstituted alkyl group;
each occurrence of n₁ is independently 0, 1, 2, 3, 4, 5, 6, 7, 8, 9, 10, 11, 12, 13, 14, 15, or 16;
each occurrence of n₂ is independently 1, 2, 3, 4, 5, 6, 7, 8, or 9;
each occurrence of n₃ is independently 0 or 1;
each occurrence of n₄ is independently, 1, 2, 3, 4, 5, 6, 7, 8, 9, 10, 11, 12, 13, 14, 15, or 16;
each occurrence of n₅ is independently 0 or 1;
each occurrence of R_{AA} is independently a proteinogenic or non-proteinogenic amino acid side chain;
each occurrence of X is independently (—NH—), (—O—), or absent;
each occurrence of X′ is independently (—NH—), (—O—), or absent;
each occurrence of Y is independently 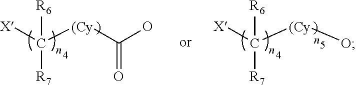
and
each occurrence of Cy is independently a 5- or 6-membered cycloalkyl, 5- or 6-membered heterocycle, 5- or 6-membered aryl, or 5- or 6-membered heteroaryl, wherein Cy optionally has fused thereto a second ring which is a 5- or 6-membered heterocycle, 5- or 6-membered cycloalkyl 5- or 6-membered aryl or a 5- or 6-membered heteroaryl ring.

32. The compound of claim 1, wherein the formula is Formula 1g: 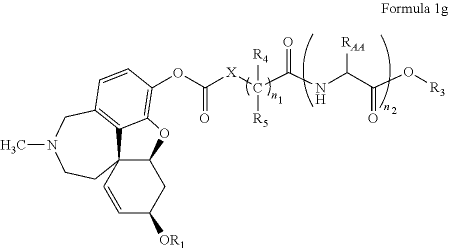
or a pharmaceutically acceptable salt thereof, wherein
R₁ is H, 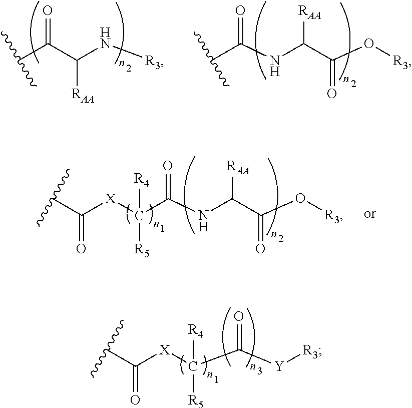
each occurrence of R₃ is independently hydrogen, a substituted alkyl group or an unsubstituted alkyl group;
each occurrence of R₄, R₅, R₆, and R₇ is independently selected from hydrogen, 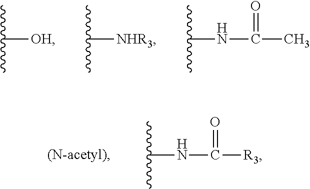
a substituted alkyl group, or an unsubstituted alkyl group;
each occurrence of n₁ is independently 0, 1, 2, 3, 4, 5, 6, 7, 8, 9, 10, 11, 12, 13, 14, 15, or 16;
each occurrence of n₂ is independently 0, 1, 2, 3, 4, 5, 6, 7, 8, or 9;
each occurrence of n₃ is independently 0 or 1;
each occurrence of n₄ is independently 0, 1, 2, 3, 4, 5, 6, 7, 8, 9, 10, 11, 12, 13, 14, 15, or 16;
each occurrence of n₅ is independently 0 or 1;
each occurrence of R_{AA} is independently a proteinogenic or non-proteinogenic amino acid side chain;
each occurrence of X is independently (—NH—), (—O—), or absent;
each occurrence of X′ is independently (—NH—), (—O—), or absent;
each occurrence of Y is independently 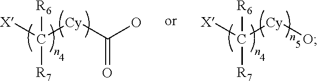
and
each occurrence of Cy is independently a 5- or 6-membered cycloalkyl, 5- or 6-membered heterocycle, 5- or 6-membered aryl, or 5- or 6-membered heteroaryl, wherein Cy optionally has fused thereto a second ring which is a 5- or 6-membered heterocycle, 5- or 6-membered cycloalkyl 5- or 6-membered aryl or a 5- or 6-membered heteroaryl ring.

33. The compound of claim 1, wherein the formula is Formula 1h 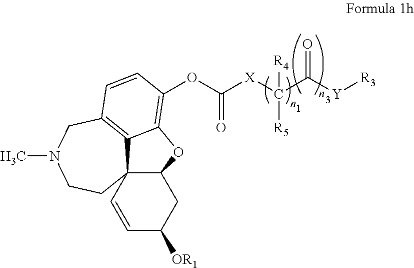
or a pharmaceutically acceptable salt thereof, wherein
R₁ is H, 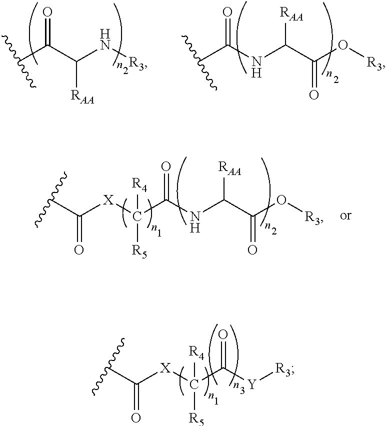
each occurrence of R₃ is independently hydrogen, a substituted alkyl group or an unsubstituted alkyl group;
each occurrence of R₄, R₅, R₆, and R₇ is independently selected from hydrogen, 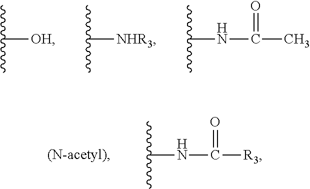
a substituted alkyl group, or an unsubstituted alkyl group;
each occurrence of n₁ is independently 0, 1, 2, 3, 4, 5, 6, 7, 8, 9, 10, 11, 12, 13, 14, 15, or 16;
each occurrence of n₂ is independently 0, 1, 2, 3, 4, 5, 6, 7, 8, or 9;
each occurrence of n₃ is independently 0 or 1;
each occurrence of n₄ is independently 0, 1, 2, 3, 4, 5, 6, 7, 8, 9, 10, 11, 12, 13, 14, 15, or 16;
each occurrence of n₅ is independently 0 or 1;
each occurrence of R_{AA} is independently a proteinogenic or non-proteinogenic amino acid side chain;
each occurrence of X is independently (—NH—), (—O—), or absent;
each occurrence of X′ is independently (—NH—), (—O—), or absent;
each occurrence of Y is independently 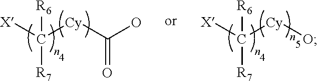
and
each occurrence of Cy is independently a 5- or 6-membered cycloalkyl, 5- or 6-membered heterocycle, 5- or 6-membered aryl, or 5- or 6-membered heteroaryl, wherein Cy optionally has fused thereto a second ring which is a 5- or 6-membered heterocycle, 5- or 6-membered cycloalkyl 5- or 6-membered aryl or a 5- or 6-membered heteroaryl ring.

34. The compound of claim 1, wherein the formula is Formula 47: 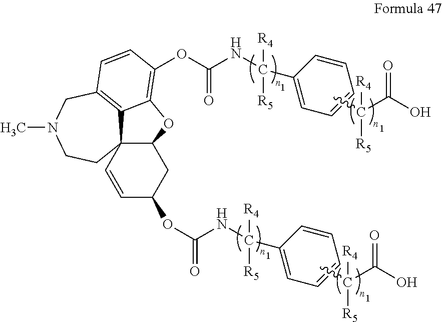
or a pharmaceutically acceptable salt thereof, wherein each occurrence of R₄, and R₅ is independently selected from hydrogen, 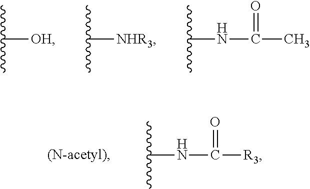
a substituted alkyl group, or an unsubstituted alkyl group; and
each n₁ is independently 0, 1, 2, 3, 4, 5, 6, 7, 8, 9, 10, 11, 12, 13, 14, 15, or 16;

35. The compound of claim 1, wherein the formula is Formula 48: 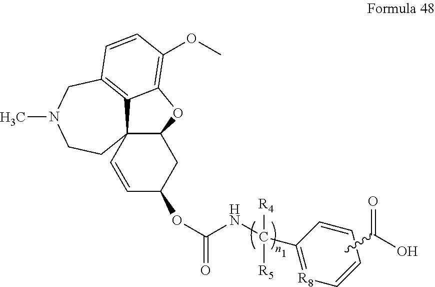
or a pharmaceutically acceptable salt thereof, wherein each occurrence of R₄ and R₅ is independently selected from hydrogen, 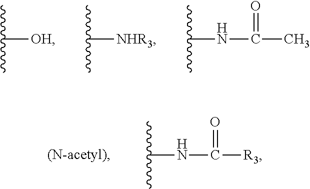
a substituted alkyl group, or an unsubstituted alkyl group; R₈ is C or N; and each occurrence of n₁ is independently from 0, 1, 2, or 3.

36. The compound of claim 1, wherein the formula is Formula 49: 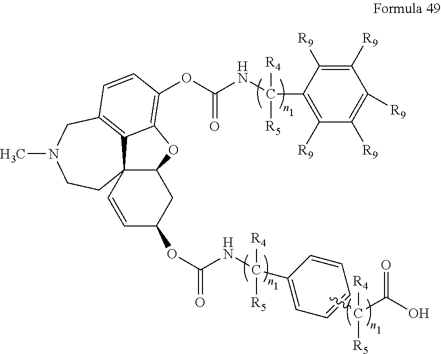
or a pharmaceutically acceptable salt thereof wherein each occurrence of R₄ and R₅ is independently selected from hydrogen, 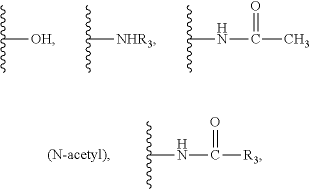
a substituted alkyl group, or an unsubstituted alkyl group;
each occurrence of R₉ is independently defined as hydrogen or 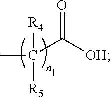
and each occurrence of n₁ can be independently be 0, 1, 2, or 3.

37. The compound of claim 1, wherein the formula is Formula 50: 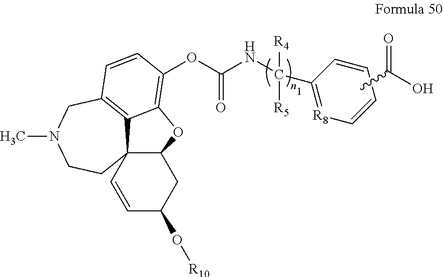
or a pharmaceutically acceptable salt thereof, wherein
each occurrence of R₄ and R₅ is independently selected from hydrogen, 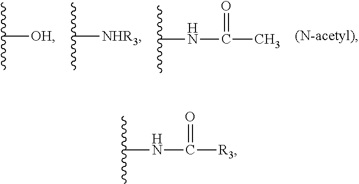
a substituted alkyl group, or an unsubstituted alkyl group;
each occurrence of R₈ is independently C or N;
R₁₀ is hydrogen or 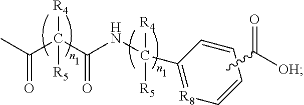
and each occurrence of n₁ can be independently be 0, 1, 2, or 3.

38. The compound of claim 1, wherein the compound exhibits lower adverse gastrointestinal side effects as compared to galantamine.

39. The compound of claim 38, wherein the gastrointestinal side effect is emesis, nausea, abdominal discomfort, diarrhea, or a combination thereof.

40. A pharmaceutical composition comprising the compound of claim 1 and one or more pharmaceutically acceptable excipients.

41. The pharmaceutical composition of claim 40, wherein the composition exhibits lower adverse gastrointestinal side effects as compared to galantamine.

42. The pharmaceutical composition of claim 41, wherein the gastrointestinal side effect is emesis, nausea, abdominal discomfort, diarrhea, or a combination thereof.

43. A method for treating cognitive impairment caused by a disease comprising administering a compound of claim 1 to a patient suffering from said cognitive impairment.

44. The method of claim 43, where in the disease is Alzheimer's disease, vascular dementia, or autism.

45. The method of claim 44, wherein the compound is galantamine (succinyl-S-valine) ester, galantamine glutaryl PABA ester, galantamine-(S)-phenylalanine carbamate trifluoroacetate, galantamine-(S)-valine ester di-trifluoroacetate, galantamine-(S)-valine ester tartrate, galantamine-(S)-Tyrosine Carbamate Trifluoroacetate, galantamine (succinyl-5-valine) ester TFA, galantamine-succinyl ester or galantamine glutarate.

46. The method of claim 45, wherein the compound is galantamine (succinyl-S-valine) ester or galantamine glutaryl PABA ester.

47. A method for achieving a sustained plasma concentration of galantamine comprising administering the compound of claim 1.

48. A method for achieving a sustained plasma concentration of galantamine comprising administering the compound of claim 1, wherein the T_{>50% Cmax} is increased by at least 100% as compared to the T_{>50% Cmax} for the parent drug.

49. A method for achieving a sustained plasma concentration of galantamine comprising administering the compound of claim 1, wherein the T_{>50% Cmax} is increased by from about 200% to about 300% as compared to the T_{>50% Cmax} for the parent drug.
